(19) United States
(12) Patent Application Publication
(10) Pub. No.: US 2025/0382598 A1
(43) Pub. Date: Dec. 18, 2025

(54) CAS EXONUCLEASE FUSION PROTEINS AND ASSOCIATED METHODS FOR EXCISION, INVERSION, AND SITE SPECIFIC INTEGRATION

(71) Applicant: SYNGENTA CROP PROTECTION AG, BASEL (CH)

(72) Inventors: Jianping XU, Haidian District, Beijing (CN); Wan SHI, Chaoyang District, Beijing (CN)

(21) Appl. No.: 18/877,433

(22) PCT Filed: Jun. 23, 2023
(86) PCT No.: PCT/US2023/068974

§ 371 Date: Dec. 20, 2024

(30) Foreign Application Priority Data
Jun. 23, 2022 (CN) 202210718723.X

(51) Int. Cl. C12N 9/22; C12N 15/11; C12N 15/82

(52) U.S. Cl. CPC C12N 9/226; C12N 15/111; C12N 15/8213; C07K 2319/09; C12N 2310/20; C12Y 301/11

(57) ABSTRACT

Provided herein are fusion proteins and associated methods and systems for increasing the efficiency of genome editing using site-directed nucleases. The fusion proteins, systems, and methods can selectively increase desired editing outcomes (e.g., inversion, excision, and homology-directed repair). Also provided are various useful compositions for the production and use of the fusion proteins and practice of the methods.

### CROSS-REFERENCE TO RELATED APPLICATIONS

This application claims priority to China Patent Application No. 202210718723.X, filed Jun. 23, 2022, which is incorporated by reference.

### FIELD

This disclosure relates to methods to increase excision, inversion, and site-specific integration. The methods presented herein are applicable to both non-homologous end joining (NHEJ) as well as homology dependent repair (HDR) mechanisms.

### SEQUENCE LISTING

This application is accompanied by a sequence listing entitled 82439-ST26.xml which is approximately 252 kilobytes in size. This sequence listing is incorporated herein by reference in its entirety.

### BACKGROUND

Site directed nucleases (SDNs) (e.g. zinc finger nucleases, transcription activator-like effector nucleases, CRISPR-associated nucleases) have gained increasing popularity in the gene editing space. These SDNs act as endonucleases and generally create double-stranded breaks (DSBs) in specific DNA sequences, activating intrinsic repair mechanisms of the cell (e.g., homologous recombination). During the repair process, site-directed modification to said specific DNA sequence can be achieved. The CRISPR (Clustered Regularly Interspaced Short Palindromic Repeats)/Cas (CRISPR-associated) system evolved in bacteria and archaea as an adaptive immune system to defend against viral attack. In recent years, the CRISPR/Cas system has attracted particular interest as a tool for genome editing. CRISPR/Cas systems that generate site-specific double stranded breaks (DSBs) can be used to edit DNA in eukaryotic cells, e.g., by producing deletions, insertions, and/or changes in nucleotide sequence.

Site-directed modifications induced by SDNs often lack precision (e.g., off-target edits may occur), and they often occur at a low frequency. For example, where a CRISPR/Cas system is configured to cause deletions by making one or more DSBs, the size of the deletion may vary, and the frequency of desired deletion events may be comparatively low. As such, there is a need for methods of increasing the efficiency of targeted genome editing using SDNs.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are fusion proteins comprising a site-directed nuclease linked to a nonspecific end-processing enzyme. In some embodiments, the site-directed nuclease comprises a CRISPR-associated nuclease. In some embodiments, the CRISPR-associated nuclease is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof. In some embodiments, the CRISPR-associated nuclease is a Cas9 enzyme. In some embodiments, the CRISPR-associated nuclease is a Cas12a enzyme.

In some embodiments of the fusion proteins provided herein, the nonspecific end-processing enzyme is a nonspecific exonuclease. In some embodiments, the nonspecific exonuclease is T5Exo, Trex2, E. coli exonuclease I, exonuclease III, exonuclease T, exonuclease IX, Exonuclease X, RecJ, Pol II, Pol III ε; WRN, MRE11, APE1, VDJP, RAD1, RAD9, p53, or Trex1. In some embodiments, the nonspecific end-processing enzyme comprises an amino acid sequence having at least 90% identity to any one of SEQ ID NOs: 4, 5, 18, 19, 20, 22, or 58-74. In some embodiments, the nonspecific end-processing enzyme is a monomer of a protein that dimerizes.

In some embodiments of the fusion proteins provided herein, the fusion protein comprises a linker located between the site-directed nuclease and the nonspecific end-processing enzyme. In some embodiments, the linker comprises SEQ ID NO:7. In some embodiments, the fusion protein comprises a nuclear localization signal. In some embodiments, the fusion protein comprises an amino acid sequence having at least 90% identity to any one of SEQ ID NOs: 50-57.

Also provided herein are recombinant nucleic acids encoding any of the fusion proteins described herein. Also provided are DNA constructs comprising a promoter operably linked to a recombinant nucleic acid described herein. In some embodiments, the promoter is at least one of an inducible promoter, a constitutive promoter, an egg cell-specific promoter, a pollen-specific promoter, or an apical meristem tissue-specific promoter. In some embodiments, the promoter is a ubiquitin 4 promoter, an actin promoter, a tubulin promoter, a MADS box promoter, or a plant virus promoter. Also provided herein are vectors comprising a recombinant nucleic acid or a DNA construct described herein. Also provided herein are cells comprising a recombinant nucleic acid, DNA construct, or vector described herein. In some embodiments, the cells are plant cells. In some embodiments, the plant cells are maize plant cells, soybean plant cells, rice plant cells, wheat plant cells, and/or sunflower plant cells.

Also provided herein are methods of editing a nucleic acid, the method comprising providing at least one fusion protein described herein; providing the nucleic acid, wherein the nucleic acid comprises a first binding site, a second binding site, and a target region comprising a portion of the nucleic acid, wherein the first binding site is adjacent to a 5′ end of the target region and the second binding site is adjacent to the 3′ end of the target region; and contacting the nucleic acid with the at least one fusion protein, wherein the at least one fusion protein specifically binds to the first binding site and the second binding site, thereby resulting in an edit to the target region of the nucleic acid. In some embodiments, the site-directed nuclease of the at least one fusion protein comprises a CRISPR-associated nuclease and the method further comprises providing at least one first guide RNA and at least one second guide RNA, wherein the at least one first guide RNA comprises a nucleotide sequence having complementarity to the first binding site and the at least one second guide RNA comprises a nucleotide sequence having complementarity to the second binding site. In some embodiments, the first binding site and the second binding site are on the same strand. In some embodiments, the first binding site and the second binding site are on opposite strands. In some embodiments, at least one of the first binding site or the second binding site are within the target region. In some embodiments, both the first binding site and the second binding site are within the target region. In some embodiments, neither the first binding site nor the second binding site are within the first target region.

In some embodiments of the methods of editing a nucleic acid provided herein, the methods further comprise providing a donor nucleic acid, wherein the donor nucleic acid comprises a third binding site, a fourth binding site, and a donor nucleotide region, wherein the third binding site is adjacent to a 5′ end of the donor nucleotide region and the fourth binding site is adjacent to the 3′ end of the donor nucleotide region and wherein the at least one fusion protein specifically binds to the third binding site and the fourth binding site. In some embodiments, the site-directed nuclease of the at least one fusion protein comprises a CRISPR-associated nuclease and the method further comprises providing at least one third guide RNA and at least one fourth guide RNA, wherein the at least one third guide RNA comprises a nucleotide sequence having complementarity to the third binding site and the at least one fourth guide RNA comprises a nucleotide sequence having complementarity to the fourth binding site. In some embodiments, the third binding site and the fourth binding site are on the same strand. In some embodiments, the third binding site and the fourth binding site are on opposite strands. In some embodiments, at least one of the third binding site or the fourth binding site are within the donor nucleotide region. In some embodiments, both the third binding site and the fourth binding site are within the donor nucleotide region. In some embodiments, neither the third binding site nor the fourth binding site are within the donor nucleotide region.

In some embodiments of the methods of editing a nucleic acid provided herein, the nucleic acid is a portion of a first chromosome. In some embodiments, the donor nucleic acid is a portion of a donor template. In some embodiments, the donor template is part of a plasmid or linear nucleic acid.

In some embodiments of the methods of editing a nucleic acid provided herein, the edit is an excision, an inversion, or a replacement of at least a portion of the target region.

In some embodiments of the methods of editing a nucleic acid provided herein, the donor nucleic acid is a portion of a second chromosome. In some embodiments, the first chromosome and the second chromosome are homologous chromosomes or non-homologous chromosomes. In some embodiments, the edit is a chromosomal rearrangement or a replacement of at least a portion of the target region. In some embodiments, the chromosomal rearrangement is a reciprocal translocation or a non-reciprocal translocation.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the stepwise cutting and inversion of a target polynucleotide sequence, according to aspects of this disclosure. Two fusion proteins bind to a first binding site and a second binding site on the same strand of a target DNA sequence. The first binding site is adjacent to the 5′ end of the target region (i.e., the region between the nuclease cleavage sites), and the second binding site is adjacent to the 3′ end of the target region. For illustrative purposes, the fusion protein in this embodiment comprises LbCas12a as the SDN linked to a Trex2 exonuclease as the nonspecific end-processing enzyme. Within the target DNA sequence, for example a gene sequence further comprising a promoter, a first exon, a first intron, etc., is the first binding site which comprises a PAM sequence located in the promoter of the gene, and the second binding site comprises a PAM sequence located in intron 1 of the gene. LbCas12a (i.e., the SDN of the fusion proteins) cleaves the DNA in the 3′ direction from the PAM and the fusion proteins remain bound to the binding sites on the cleaved polynucleotide ends that comprise the PAM. Because the first binding site (indicated by “gRNA 1”) is upstream of the cleavage site (i.e., outside of the target region), the fusion protein bound to the first binding site remains bound to the cleaved polynucleotide end outside of and upstream from the target region. Because the second binding site (indicated by “gRNA 2”) is upstream of the cleavage site (i.e., inside the target region), the fusion protein bound to the second binding site remains bound to the downstream end of the target region. Without being bound by any particular theory, the dimerization of the fusion proteins (i.e., through dimerization of Trex2) bound to the first and second binding sites will bring the downstream end of the target region and the end of the polynucleotide upstream of the target region into close proximity, where they will be joined through a DNA repair mechanism such as NHEJ or MMEJ. Joining of the remaining polynucleotide ends (i.e., those that are not bound by a fusion protein) through DNA repair mechanisms thus results in an inversion of the target region.

FIG. 2 shows the stepwise cutting and excision of a target polynucleotide sequence, according to aspects of this disclosure. Two fusion proteins bind to a first binding site and a second binding site on opposite strands of a DNA polynucleotide. The first binding site is adjacent to the 5′ end of the target region, and the second binding site is adjacent to the 3′ end of the target region. For illustrative purposes, the fusion protein in this embodiment comprises LbCas12a as the SDN linked to a Trex2 exonuclease as the nonspecific end-processing enzyme. In this illustration, the DNA polynucleotide is a gene; the first binding site comprises a PAM sequence located in the promoter of the gene, and the second binding site comprises a PAM sequence located in intron 1 of the gene. In this illustration, LbCas12a (i.e., the SDN of the fusion protein) cleaves the polynucleotide in the 3′ direction from the PAM and the fusion proteins remain bound to the binding sites on the cleaved polynucleotide ends that comprise the PAM. Because the first binding site (indicated by “gRNA 1”) is upstream of the cleavage site (i.e., outside of the target region), the fusion protein bound to the first binding site remains bound to the cleaved polynucleotide end outside of and upstream from the target region. Because the second binding site (indicated by “gRNA 2”) is downstream of the cleavage site (i.e., outside the target region), the fusion protein bound to the second binding site remains bound to the cleaved polynucleotide end outside of and downstream from the target region. Without being bound by any particular theory, the dimerization of the fusion proteins bound to the first and second binding sites will bring the cleaved polynucleotide ends upstream of and downstream of the target region into close proximity, where they will be joined through a DNA repair mechanism such as NHEJ or MMEJ, resulting in excision of the target region.

FIGS. 3A and 3B show the stepwise cutting, excision, and insertion of a polynucleotide fragment at a target polynucleotide sequence, according to aspects of this disclosure. Shown are methods comprising two fusion proteins that bind to a first binding site and a second binding site on opposite strands of a DNA polynucleotide (i.e., the nucleotide to be edited, shown here as “Genomic DNA”) and two fusion proteins that bind to a third binding site and a fourth binding site on opposite strands of a donor nucleic acid (shown here as “Donor DNA”). In both embodiments, the first binding site is adjacent to the 5′ end of the target region (i.e., the region between the nuclease cleavage sites), the second binding site is adjacent to the 3′ end of the target region, the third binding site is adjacent to the 5′ end of the donor nucleotide region (i.e., the region of the donor nucleotide between the nuclease cleavage sites), and the fourth binding site is adjacent to the 3′ end of the donor nucleotide region. For illustrative purposes, the fusion protein in these embodiments comprises LbCas12a as the SDN linked to a Trex2 exonuclease as the nonspecific end-processing enzyme. LbCas12a (i.e., the SDN of the fusion proteins) cleaves the polynucleotides (i.e., both the nucleotide to be edited and the donor nucleic acid) in the 3′ direction from the PAM and the fusion proteins remain bound to the binding sites on the cleaved polynucleotide ends that comprise the PAM. Because the first binding site (indicated by “gRNA-a”) is upstream of the cleavage site (i.e., outside of the target region), the fusion protein bound to the first binding site remains bound to the cleaved polynucleotide end outside of and upstream from the target region. Because the second binding site (indicated by “gRNA-b”) is downstream of the cleavage site (i.e., outside the target region), the fusion protein bound to the second binding site remains bound to the cleaved polynucleotide end outside of and downstream from the target region. Because the third binding site (indicated by “gRNA-a/c”) is downstream of the cleavage site (i.e., within the donor nucleotide region), the fusion protein bound to the third binding site remains bound to the cleaved polynucleotide end of the donor nucleotide region (i.e., to the upstream end of the donor nucleotide region). Because the fourth binding site (indicated by “gRNA-b/d”) is upstream of the cleavage site (i.e., within the donor nucleotide region), the fusion protein bound to the fourth binding site remains bound to the cleaved polynucleotide end of the donor nucleotide region (i.e., to the downstream end of the donor nucleotide region). Without being bound by any particular theory, the dimerization of the fusion proteins (e.g., through dimerization of Trex2) bound to the first and third binding sites will bring the cleaved polynucleotide end upstream of the target region into close proximity with the upstream end of the donor nucleotide region, and the dimerization of the fusion proteins bound to the second and fourth binding sites will bring the cleaved polynucleotide end downstream of the target region into close proximity with the downstream end of the donor nucleotide region. Joining of the polynucleotide ends that are in close proximity through a DNA repair mechanism such as NHEJ or MMEJ will result in replacement of the target region with the donor nucleotide region. The donor nucleotide region may also be inserted in the reverse orientation (i.e., if the fusion proteins bound to the first and fourth binding sites dimerize and the fusion proteins bound to the second and third binding sites dimerize). In FIG. 3A, the donor nucleotide region does not comprise homology arms, so NHEJ or MMEJ repair is more likely than HDR. In FIG. 3B, the donor nucleotide region comprises homology arms. Without being bound by any particular theory, dimerization of the fusion proteins bound to the first binding site and the third binding site and dimerization of the fusion proteins bound to the second binding site and the fourth binding site will promote HDR-mediated repair by bringing the donor nucleotide region homology arms into close proximity with the homologous sequences in the nucleotide to be edited (e.g., the genomic DNA).

FIG. 4 shows the stepwise cutting and translocation of chromosomes, according to aspects of this disclosure. Two fusion proteins bind to a first binding site and a second binding site on opposite strands of a DNA polynucleotide (i.e., the nucleotide to be edited, shown here as “Recipient chromosome”) and two fusion proteins bind to a third binding site and a fourth binding site on opposite strands of a donor nucleic acid (shown here as “Donor chromosome”). The first binding site is adjacent to the 5′ end of the target region (i.e., the region between the nuclease cleavage sites), the second binding site is adjacent to the 3′ end of the target region, the third binding site is adjacent to the 5′ end of the donor nucleotide region (i.e., the region of the donor nucleotide between the nuclease cleavage sites), and the fourth binding site is adjacent to the 3′ end of the donor nucleotide region. For illustrative purposes, the fusion protein in these embodiments comprises LbCas12a as the SDN linked to a Trex2 exonuclease as the nonspecific end-processing enzyme. LbCas12a (i.e., the SDN of the fusion proteins) cleaves the polynucleotides (i.e., both the nucleotide to be edited and the donor nucleic acid) in the 3′ direction from the PAM and the fusion proteins remain bound to the binding sites on the cleaved polynucleotide ends that comprise the PAM. Because the first binding site (indicated by “gRNA-a”) is upstream of the cleavage site (i.e., outside of the target region), the fusion protein bound to the first binding site remains bound to the cleaved polynucleotide end outside of and upstream from the target region. Because the second binding site (indicated by “gRNA-b”) is downstream of the cleavage site (i.e., outside the target region), the fusion protein bound to the second binding site remains bound to the cleaved polynucleotide end outside of and downstream from the target region. Because the third binding site (indicated by “gRNA-c”) is upstream of the cleavage site (i.e., outside the donor nucleotide region), the fusion protein bound to the third binding site remains bound to the cleaved donor nucleotide end outside of and upstream from the donor nucleotide region. Because the fourth binding site (indicated by “gRNA-d”) is downstream of the cleavage site (i.e., outside the donor nucleotide region), the fusion protein bound to the fourth binding site remains bound to the cleaved donor nucleotide end outside of and downstream of the donor nucleotide region. Without being bound by any particular theory, the dimerization of the fusion proteins (e.g., through dimerization of Trex2) bound to the first and fourth binding sites will bring the cleaved polynucleotide end upstream of the target region into close proximity with the cleaved polynucleotide end downstream of the donor nucleotide region, and the dimerization of the fusion proteins bound to the second and third binding sites will bring the cleaved polynucleotide end downstream of the target region into close proximity with the cleaved polynucleotide end upstream of the donor nucleotide region. Joining of the polynucleotide ends that are in close proximity through a DNA repair mechanism such as NHEJ or MMEJ will result in a translocation of the polynucleotides (and excision of the target region and the donor nucleotide region).

FIG. 5 shows the expected outcomes of editing where the guide RNA molecules (“gRNAs”) are designed to bind on the same strand of target DNA polynucleotide sequence and in the same orientation, according to aspects of this disclosure. In this figure at the first line, the gRNAs target a sequence in the promoter and the first intron of the target gene to be edited. At the second line, the figure shows the excision of the intervening sequence between the gRNA binding sites, thus disrupting the gene. At the third line, the figure shows an alternate outcome where the intervening sequence is inverted and ligated in the reverse orientation, thus disrupting the gene.

FIG. 6 shows a comparison of Cas9 and Cas12a cutting, according to aspects of this disclosure. Compared with Cas9, it is more difficult to excise or invert the genomic sequence between paired guide RNA targeting sites using Cas 12a. This could be explained by the fact that Cas 12a generates double strand break with sticky ends, which could be more difficult to fall apart from each other, in contrast to the blunt ends generated by Cas9. In order to improve the frequencies of fragment excision and inversion, various DNA exonucleases were fused to LbCas12a to trim and disengage the sticky ends immediately after LbCas12a generates the double stand break.

FIGS. 7A and 7B show examples where two gRNAs targeting opposite strands were selected to excise the genomic fragment between the two targeting sites, according to aspects of this disclosure. In FIG. 7A, two gRNA pairs were selected to test the prediction that binding sites on opposite strands outside of the target region may further increase the excision frequency induced by LbCas12a-Trex2. To design such gRNA pairs, ZmDMR6-crRNA1 was kept and paired with two gRNAs, ZmDMR6-crRNA3 (SEQ ID NO:23) and ZmDMR6-crRNA4 (SEQ ID NO:24) respectively, of which the targets are downstream to that of ZmDMR6-crRNA2 and on the complementary strand. The expected excision with gRNA pairing ZmDMR6-crRNA1 and ZmDMR6-crRNA3 is 1233 bp. The expected excision with gRNA pairing ZmDMR6-crRNA1 and ZmDMR6-crRNA4 is 1387 bp. In FIG. 7B, one gRNA pair was selected to test if binding sites on opposite strands inside of the target region may significantly increase the excision frequency. ZmWaxy1-crRNA1 (SEQ ID NO:25) was designed to target a sequence adjacent to a TTTG PAM in Exon 4 on the coding strand, while ZmWaxy1-crRNA5 (SEQ ID NO:26) was designed to target a sequence adjacent to a TTTA PAM in the promoter region on the complementary strand. The expected excision with gRNA pairing ZmWaxy1-crRNA1 and ZmWaxy1-crRNA5 is 1.1 kb.

FIG. 8 shows the gRNA design for editing a gene in soy, according to aspects of this disclosure. The first gRNA, GmFAD2A-crRNA1 (SEQ ID NO:28) was designed to target a sequence adjacent to a TTTG PAM in the FAD2-1A promoter region on the coding strand. A second gRNA, GmFAD2A-crRNA2 (SEQ ID NO:29) was designed to target a sequence adjacent to a TTTG PAM next to the 3′ splicing site of the first intron on the coding strand. The distance between crRNA1 and crRNA2 target sites is about 1.1 kb. A third gRNA, GmFAD2A-crRNA3 (SEQ ID NO:30) was designed to target a sequence adjacent to a TTTG in the second exon on the complementary strand. The distance between crRNA1 and crRNA3 target sites is about 1.2 kb.

FIG. 9 shows the design of a donor sequence comprising left and right homology arms to facilitate HDR, according to aspects of this disclosure. A 458-bp genomic sequence (SEQ ID NO:48) upstream to the PAM of ZmSH1-crRNA2 target was selected as the left homology arm (LHA) to mediate HDR, and was added to the 5′ end of the cargo sequence. A 509-bp genomic sequence (SEQ ID NO:49) downstream to the PAM of ZmSH1-crRNA2 target was selected as the right homology arm (RHA) and was added to the 3′ end of the cargo sequence. The target sequences of ZmSH1-crRNA1 and ZmSH1-crRNA2, both with a TTTG PAM added to the 5′ end, were added to the 5′ end of the left homology arm and the 3′ end of the right homology arm, respectively and in such a way that the crRNA binding sites are on opposite strands but within the donor nucleotide region, to generate the donor DNA.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used herein, the singular forms “a”, “an” and “the” include plural referents unless the content clearly dictates otherwise. Thus, for example, reference to “an enzyme” optionally includes a combination of two or more such molecules, and the like.

As used herein, “and/or” refers to and encompasses any and all possible combinations of one or more of the associated listed items.

The term “about” as used herein refers to the usual error range for the respective value readily known to the skilled person in this technical field, for example ±20%, ±10%, or ±5%, are within the intended meaning of the recited value.

As used herein, the term “comprising” or “comprise” is open-ended. When used in connection with a subject nucleic acid (or amino acid sequence), it refers to a nucleic acid sequence (or an amino acid sequence) that includes the subject sequence as a part or as its entire sequence.

As used herein, the transitional phrase “consisting essentially of” means that the scope of a claim is to be interpreted to encompass the specified materials or steps recited in the claim and those that do not materially affect the basic and novel characteristic(s) of the claimed matter. Thus, the term “consisting essentially of” when used in a claim of this disclosure is not intended to be interpreted to be equivalent to “comprising.”

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

The term “plant” as used herein refers to any plant at any stage of development, particularly a seed plant. The term “plant cell” as used herein refers to a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. The plant cell may be derived from or part of an angiosperm or gymnosperm. The plant cell may be a monocotyledonous plant cell (e.g., a maize cell, a rice cell, a sorghum cell, a sugarcane cell, a barley cell, a wheat cell, an oat cell, a turf grass cell, or an ornamental grass cell) or a dicotyledonous plant cell (e.g., a tobacco cell, a pepper cell, an eggplant cell, a sunflower cell, a crucifer cell, a flax cell, a potato cell, a cotton cell, a soybean cell, a sugar bee cell, or an oilseed rape cell. The term “plant cell culture” as used herein refers to cultures of plant units such as, for example, protoplasts, cell culture cells, cells in plant tissues, pollen, pollen tubes, ovules, embryo sacs, zygotes and embryos at various stages of development. The term “plant tissue” as used herein refers to a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue. The term “plant part” as used herein refers to a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

The terms “polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The terms “nucleic acid” and “polynucleotide” are used interchangeably and as used herein refer to deoxyribonucleic acids (DNA) or ribonucleic acids (RNA) and polymers thereof in either single- or double-stranded form, as well as to both sense and anti-sense strands of RNA, cDNA, genomic DNA, mitochondrial DNA, and synthetic forms and mixed polymers of the above. In higher plants, DNA is the genetic material while RNA is involved in the transfer of information contained within DNA into proteins. A “genome” is the entire body of genetic material contained in each cell of an organism. It is understood that when an RNA is described, its corresponding cDNA is also described, wherein uridine is represented as thymidine. In particular embodiments, a nucleotide refers to a ribonucleotide, deoxynucleotide or a modified form of either type of nucleotide, and combinations thereof. In addition, a polynucleotide disclosed herein may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Nucleotide sequences are “complementary” when they specifically hybridize in solution (e.g., according to Watson-Crick base pairing rules). The term also includes codon-optimized nucleic acids that encode the same polypeptide sequence. It is also understood that nucleic acids can be unpurified, purified, or attached, for example, to a synthetic material such as a bead or column matrix.

The term “corresponding to” in the context of nucleic acid sequences means that when the nucleic acid sequences of certain sequences are aligned with each other, the nucleic acids that “correspond to” certain enumerated positions in the present invention are those that align with these positions in a reference sequence, but that are not necessarily in these exact numerical positions relative to a particular nucleic acid sequence of the invention. Optimal alignment of sequences for comparison can be conducted by computerized implementations of known algorithms. or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. See Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994).

The terms “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix. See Henikoff & Henikoff, Proc. Natl. Acad. Sci. USA 89:10915 (1989).

The BLAST algorithm also performs a statistical analysis of the similarity between two sequences. See, e.g., Karlin & Altschul, Proc. Nat'l. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Recombination” is the exchange of DNA strands to produce new nucleotide sequence arrangements. The term may refer to the process of homologous recombination that occurs in double-strand DNA break repair, where a polynucleotide is used as a template to repair a homologous polynucleotide. The term may also refer to exchange of information between two homologous chromosomes during meiosis. The frequency of double recombination is the product of the frequencies of the single recombinants. For instance, a recombinant in a 10 cM area can be found with a frequency of 10%, and double recombinants are found with a frequency of 10%×10%=1% (1 centimorgan is defined as 1% recombinant progeny in a testcross).

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. Genes can include both coding and non-coding regions (e.g., introns, regulatory elements, promoters, enhancers, termination sequences and 5′ and 3′ untranslated regions). A gene typically expresses mRNA, functional RNA, or specific protein, including regulatory sequences. Genes may or may not be capable of being used to produce a functional protein. In some embodiments, a gene refers to only the coding region. The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

A “gene of interest” or “nucleotide sequence of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

“Homology dependent repair” or “homology directed repair” or “HDR” refers to a mechanism for repairing ssDNA and double stranded dna (dsDNA) damage in cells. This repair mechanism can be used by the cell when there is an HDR template with a sequence with significant homology to the injury site. The term “perfect HDR” refers to a situation in which genomic-homology junctions in the replaced allele underwent complete HDR and “imperfect HDR” refers to a situation in which genomic-homology junctions in the replaced allele underwent partial or incomplete HDR. a donor DNA molecule with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule. In some cases, a donor DNA molecule is introduced into a cell. In some cases, at least a segment of a donor DNA molecule integrates into the genome of the cell.

“Microhomology-mediated end joining” or “MMEJ” or “alternative nonhomologous end-joining” (Alt-NHEJ) refers to a form of repairing double-stranded breaks in DNA. This repair mechanism utilizes microhomologous sequences to align the broken strands. “Non-homologous end joining” or “NHEJ” refers to a form of repairing double-stranded breaks in DNA. The double-strand breaks are repaired by direct ligation of the break ends to one another. Generally, no new nucleic acid material is inserted into the site, although some nucleic acid material may be lost or added, resulting in a small deletion or a small insertion.

### II. Introduction

Provided herein are fusion proteins and associated recombinant nucleic acids, systems, and methods to increase the efficiency of genome editing using SDNs, through inversion, excision, and HDR using fusion proteins and donor DNA tethering methods. The disclosure is based in part on the discovery by the inventors that fusion of a SDN to a nonspecific end-processing enzyme (e.g., a nonspecific exonuclease) results in increased frequency of desirable editing outcomes, such as inversion of a genome fragment between two targeted SDN-induced double strand breaks (DSBs), as demonstrated in Example 1 herein. It is generally thought that the particular cellular mechanism used for DSB repair depends on the nature of the DNA ends produced by the DSB (e.g., blunt ends vs. sticky ends) and/or the level of end-processing that occurs (e.g., whether one of the strands is resected). A DSB in which no end resection occurs is generally repaired by classical non-homologous end joining (C-NHEJ). C-NHEJ is considered an “error-prone” pathway as it leads, in some cases, to the formation of small insertions and deletions. However, if end resection does take place, the ends of a DSB may include one or more overhangs (e.g., 3′ overhangs or 5′ overhangs), which can interact with nearby homologous sequences. The mechanism by which the DSB is repaired may vary depending on the extent of processing. When the ends of a DSB undergo relatively limited end resection, the DSB is generally processed by alternative non-homologous end joining (ALT-NHEJ). ALT-NHEJ refers to a class of pathways that includes blunt end-joining (blunt EJ) and microhomology mediated end joining (MMEJ) which tend to result in deletions, as well as synthesis dependent micro homology mediated end joining (SD-MMEJ), which tends to result in insertions. However, when end resection is extensive, the resulting overhangs may undergo strand invasion of highly homologous sequences (which can be endogenous sequences or heterologous sequences), followed by repair of the DSB by a homology-dependent recombination (HDR) pathway. Without being bound by any particular theory, it is possible that the fusion proteins provided herein increase the frequency of desirable editing outcomes by coupling DSB-formation with the desired type and/or level of end-processing to bias DSB repair toward a particular pathway.

The present disclosure is also based in part on the discovery by the inventors that use of fusion proteins which are able to dimerize can increase the frequency of desirable editing outcomes. As discussed further herein, the fusion proteins are able to remain bound to their nucleic acid target following DSB formation. Further, fusion proteins can be targeted to remain bound to a portion of the nucleic acid target upstream or downstream of the DSB cleavage site. When two or more fusion proteins that are able to form dimers are used, the polynucleotide ends to which the fusion proteins are bound are brought into close proximity. Without being bound by any particular theory, it is possible that this close proximity influences the likelihood that a particular DSB repair pathway will be used. Additionally, the inventors have shown that is is possible to bias DSB repair toward different results (e.g., excision of a target fragment, inversion of a target fragment, or HDR using a donor template) by modulating the targeting of fusion proteins.

### III. Fusion Proteins

In one aspect, provided herein are fusion proteins comprising a site-directed nuclease linked to a nonspecific end-processing enzyme. As used throughout, a “fusion protein” is a protein comprising two different polypeptide sequences, i.e. a site-directed nuclease polypeptide sequence and a nonspecific end-processing enzyme polypeptide sequence, that are joined or linked to form a single polypeptide. In some embodiments, the two amino acid sequences are encoded by separate nucleic acid sequences that have been joined so that they are transcribed and translated to produce a single polypeptide. The site-directed nuclease and the nonspecific end-processing enzyme can be linked in any order and orientation relative to each other. For example, the C′ terminal end of the site-directed nuclease may be linked to the N′ terminal end or the C′ terminal end of the nonspecific end-processing enzyme. The site-directed nuclease and the nonspecific end-processing enzyme may also be separated by one or more additional fusion protein domains, as described below.

### A. Site-Directed Nuclease

The fusion proteins provided herein comprise a site-directed polypeptide. A site-directed modifying polypeptide modifies target DNA (e.g., via cleavage or methylation of target DNA) and/or a polypeptide associated with target DNA (e.g., methylation or acetylation of a histone tail). In some embodiments, a site-directed modifying polypeptide interacts with a guide RNA, which is either a single RNA molecule or a RNA duplex of at least two RNA molecules, and is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with the guide RNA. In some embodiments, the site-directed polypeptide is a site-directed nuclease, which is able to cleave one or both strands of DNA at a specified target sequence.

The term “cleavage” or “cleaving” refers to breaking of the covalent phosphodiester linkage in the ribosylphosphodiester backbone of a polynucleotide and encompass both single-stranded breaks and double-stranded breaks. Double-stranded cleavage can occur as a result of two distinct single-stranded cleavage events. Cleavage can result in the production of either blunt ends or staggered ends (also known as sticky ends). A “nuclease cleavage site” or “genomic nuclease cleavage site” is a region of nucleotides within which a site-directed nuclease cleaves (e.g., when bound to a proximal binding site). When the polynucleotide is DNA (e.g., genomic DNA), one or both strands can be cleaved at the nuclease cleavage site. Such cleavage by the nuclease enzyme initiates DNA repair mechanisms within the cell, which establishes an environment for homologous recombination to occur.

Various site-directed nucleases can be used in the fusion proteins, systems, and methods disclosed herein. Suitable nucleases include, but are not limited to, CRISPR-associated (Cas) proteins or Cas nucleases; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo); Adenosine deaminases acting on RNA (ADAR); CRISPR-Cas-inspired RNA targeting (CIRT) system; Pumilio/fem-3 binding factor (PUF), homing endonuclease, or any functional fragment thereof, any derivative thereof; any variant thereof; and any fragment thereof. Exemplary site-directed nucleases suitable for use in the fusion proteins, systems, and methods disclosed herein are described further below.

In some embodiments, the site-directed nuclease is a naturally-occurring site-directed nuclease. Exemplary naturally-occurring site-directed nucleases are known in the art (see for example, Makarova et al., 2017, Cell 168:328-328.e1, and Shmakov et al., 2017, Nat Rev Microbiol 15 (3): 169-182, both herein incorporated by reference). In some embodiments, a site-directed nuclease binds a DNA-targeting polynucleotide (e.g., a guide RNA) and is thereby directed to a specific sequence within a target DNA and cleaves the target DNA.

In some embodiments, the site-directed nuclease is modified from its natural sequence (e.g., via mutation or one or more amino acid residues) to change its function. For example, the site-directed nuclease may be modified to be enzymatically inactive. The term “enzymatically inactive” can refer to a site-directed nuclease that can bind to a nucleic acid sequence in a polynucleotide in a sequence-specific manner, but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a wild-type exemplary activity (e.g., nucleic acid cleaving activity, wild-type Cas9 activity).

In some embodiments, the site-directed nuclease (e.g., an enzymatically inactive site-directed nuclease) is fused to one or more transcription repressor domains, activator domains, epigenetic domains, recombinase domains, transposase domains, flippase domains, nickase domains, cleavage domains, or any combination thereof. The activator domain can include one or more tandem activation domains located at the carboxyl terminus of the enzyme. In other cases, the actuator moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-KB p65 subunit, Epstein-Barr virus R transactivator (Rta) and are described in Chavez et al., Nat Methods, 2015, 12 (4): 326-328 and U.S. Patent App. Publ. No. 20140068797. Non-limiting exemplary repression domains include the KRAB (Kruppel-associated box) domain of Koxl, the Mad mSIN3 interaction domain (SID), ERF repressor domain (ERD), and are described in Chavez et al., Nat Methods, 2015, 12 (4): 326-328 and U.S. Patent App. Publ. No. 20140068797. A nuclease can also be fused to a heterologous polypeptide providing increased or decreased stability. The fused domain or heterologous polypeptide can be located at the N-terminus, the C-terminus, or internally within the nuclease.

### CRISPR/Cas Nucleases

In some embodiments, the site-directed nuclease comprises a CRISPR-associated (Cas) protein or a Cas nuclease that functions in a CRISPR (Clustered Regularly Interspaced Short Palindromic Repeats)/Cas system. In bacteria, this system can provide adaptive immunity against foreign DNA (Barrangou, R., et al, “CRISPR provides acquired resistance against viruses in prokaryotes, “Science (2007) 315:1709-1712; Makarova, K. S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39:9275-9282). In a wide variety of organisms including diverse mammals, animals, plants, microbes, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (2011) 471:602-607). DNA cleavage (e.g., double-strand breaks) can result in DNA break repair which allows for the introduction of gene modification(s) (e.g., nucleic acid editing). DNA break repair can occur via non-homologous end joining (NHEJ), microhomology-mediated end joining (MMEJ), or homology-directed repair (HDR). In some embodiments, donor nucleic acids are used to promote HDR, as detailed below in the “Systems” section. CRISPR-Cas systems have been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31:233-239; Sander, J. D. & Joung, J. K, “CRISPR-Cas systems for editing, regulating and targeting genomes,” Nature Biotechnol. (2014) 32:347-355).

In some embodiments, the site-directed nuclease described herein comprises a Cas protein that forms a complex with a guide nucleic acid, such as a guide RNA (described further below in the “Systems” section). In some embodiments, the site-directed nuclease comprises a Cas protein that forms a complex with a single guide nucleic acid, such as a single guide RNA (sgRNA). In some embodiments, the site-directed nuclease comprises a RNA-binding protein (RBP) optionally complexed with a guide nucleic acid, such as a guide RNA (e.g., sgRNA), which is able to form a complex with a Cas protein. In some instances, RNA-guided Cas proteins recognize DNA targets that are complementary to a portion of the gRNA known as a CRISPR RNA (crRNA) sequence. The target sequence is often referred to as a protospacer, and the part of the crRNA sequence that is complementary to the protospacer is often referred to as a spacer. In order to function (e.g., to cleave DNA), many Cas nucleases also require a specific protospacer adjacent motif (PAM), an approximately 2 to 6 base pair DNA sequence immediately following the protospacer sequence.

Various site-directed Cas nucleases (e.g., Cas proteins from different species) may be useful in the fusion proteins, systems, and methods provided herein based on the various enzymatic characteristics of the different Cas proteins (e.g., different protospacer adjacent motif (PAM) sequence preferences; increased or decreased enzymatic activity; increased or decreased level of cellular toxicity; propensity to result in one or more of NHEJ, homology-directed repair, single strand breaks, double strand breaks, etc.). Cas proteins from various species (for example, those disclosed in Shmakov et al., 2017, or polypeptides derived therefrom) may require different PAM sequences in the target DNA. Thus, for a particular Cas enzyme of choice, the PAM sequence requirement may be different than the 5′-N GG-3′ sequence (where N is either a A, T, C, or G) known to be required for Cas9 activity. Many Cas9 orthologues from a wide variety of species have been identified, and the proteins share only a few identical amino acids. All identified Cas9 orthologs have the same domain architecture with a central HNH endonuclease domain and a split RuvC/RNaseH domain. Cas9 proteins share 4 key motifs with a conserved architecture; Motifs 1, 2, and 4 are RuvC like motifs, while motif 3 is an HNH-motif. In contrast, Cas12a proteins from various species may have differing PAM sequence requirements compared to the LbCas12a canonical PAM of TTTV.

Any suitable CRISPR/Cas system can be used. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8al, Cas8a2, Cas8b, Cas8c, Cas9 (Csnl or Csx12), Cas10, Cas10d, CasF, CasG, CasH, Cpf1, Csy1, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, and Cul966, and homologs or modified versions thereof. In some embodiments, the site-directed nuclease of the fusion proteins provided herein comprises a CRISPR-associated nuclease, wherein the CRISPR-associated nuclease is Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, or Cas14. In some embodiments, the CRISPR-associated nuclease is a Cas9 enzyme. In some embodiments, the CRISPR-associated nuclease is a Cas12a enzyme. In some embodiments, the CRISPR-associated nuclease is a nickase or deactivated version of a CRISPR-associated nuclease.

Lachnospiraceae bacterium Cpf1 (LbCpf1) is one of many Cpf1 proteins of a large group. The terms “Cpf1” and “Cas12a” are used interchangeably throughout this disclosure. Cpf1 is a Cas protein. In some embodiments, site-directed nuclease is a catalytically inactive Cas12a from Lachnospiraceae bacterium (“dLbCas12a”). In other embodiments, the site directed nuclease is catalytically active Cas12a from Lachnospiraceae bacterium (“LbCas12a”) or Moraxella bovoculi AAX08_00205 (“Mb2Cas12a”). In some embodiments, the site-directed nuclease domain of the fusion protein is a Cas12a protein from any of Lachnospiraceae bacterium, Acidaminococcus sp., Moraxella bovoculi, Thiomicrospira sp., Moraxella lacunata, Methanomethylophilus alvus, Btyrivibrio sp., or Bacteroidetesoral sp.

A Cas protein can comprise one or more domains. Non-limiting examples of domains include guide nucleic acid recognition and/or binding domains, nuclease domains (e.g., DNase or RNase domains, RuvC, HNH), DNA binding domains, RNA binding domains, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

A Cas protein used herein can be an active variant, inactive variant, or fragment of a wild-type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild-type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type or modified Cas protein or a portion thereof. Variants or fragments can be targeted to a nucleic acid locus in complex with a guide nucleic acid while lacking nucleic acid cleavage activity.

In some embodiments, a modified Cas protein has decreased function relative to the unmodified form. In some embodiments, a modified Cas protein is deficient in a function of the unmodified form. For example, a nuclease deficient Cas protein retains the ability to bind DNA but lacks or has reduced nucleic acid cleavage activity. A Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products. In some embodiments, the modified Cas protein has no more than 90%, no more than 80%, no more than 70%, no more than 60%, no more than 50%, no more than 40%, no more than 30%, no more than 20%, no more than 10%, no more than 5%, or no more than 1% of the function (e.g., nuclease activity) of the wild-type Cas protein (e.g., Cas9 from S. pyogenes). In some embodiments, the modified Cas protein has no substantial function of the wild-type Cas protein. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein or a dead Cas 12a protein.

In some embodiments, a modified Cas protein can be a modified Cas “base editor”. Base editing enables direct, irreversible conversion of one target DNA base into another in a programmable manner, without requiring DNA cleavage or a donor DNA molecule. For example, Komor et al (2016, Nature, 533:420-424), teach a Cas9-cytidine deaminase fusion, where the Cas9 has also been engineered to be inactivated and not induce double-stranded DNA breaks. Additionally, Gaudelli et al (2017, Nature, doi: 10. 1038/nature24644) teach a catalytically impaired Cas9 fused to a tRNA adenosine deaminase, which can mediate conversion of an A/T to G/C in a target DNA sequence. Another class of engineered Cas9 nucleases that can be used as the site-directed nuclease in the fusion proteins of this disclosure are variants which can recognize a broad range of PAM sequences, including NG, GAA, and GAT (Hu et al., 2018, Nature, doi: 10.1038/nature26155).

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. In some embodiments, double strand break targeting specificity is improved by targeting a nickase to opposite strands at two nearby loci. If a nickase cleaves the single strand at both loci, a double strand break is formed and can be repaired via HR as described herein. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA.

### Zinc Finger Nucleases

In some embodiments, a site-directed nuclease suitable for use in the fusion proteins or methods described herein is a “zinc finger nuclease” or “ZFN.” ZFNs refer to a fusion between a cleavage domain, such as a cleavage domain of Fokl, and at least one zinc finger motif (e.g., at least 2, 3, 4, or 5 zinc finger motifs) which can bind polynucleotides such as DNA and RNA. The heterodimerization at certain positions in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9 (8): 805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Publication Nos. 2003/0232410 and 2009/0203140.

In some embodiments, a nuclease comprising a ZFN can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. In some embodiments, a ZFN is a zinc finger nickase which induces site-specific single-strand DNA breaks or nicks, thus resulting in HR. Descriptions of zinc finger nickases are found, e.g., in Ramirez et al., Nucl Acids Res, 2012, 40 (12): 5560-8; Kim et al., Genome Res, 2012, 22 (7): 1327-33. In some embodiments, a ZFN binds a polynucleotide (e.g., DNA and/or RNA) but is unable to cleave the polynucleotide.

In some embodiments, the cleavage domain of a nuclease comprising a ZFN comprises a modified form of a wild-type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type cleavage domain. The modified form of the cleavage domain can have no substantial nucleic acid-cleaving activity. In some embodiments, the cleavage domain is enzymatically inactive.

### TAL-Effector Nucleases

In some embodiments, a site-directed nuclease suitable for use in the fusion proteins, systems, or methods described herein is a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fok1. Several mutations to Fok1 have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HR. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. In some cases, a single-stranded donor DNA repair template is provided to promote HR. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13 (4): 291-303; Gaj et al., Nat Methods, 2012, 9 (8): 805-7; Beurdeley et al., Nat Commun, 2013, 4:1762; and Joung and Sander, Nat Rev Mol Cell Biol, 2013, 14 (1): 49-55.

In some embodiments, a TALEN is engineered for reduced nuclease activity. In some embodiments, the nuclease domain of a TALEN comprises a modified form of a wild-type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. A non-limiting example of a transcriptional repression domain includes a Kruppel-associated box domain.

### Meganucleases

In some embodiments, a site-directed nuclease suitable for use in the fusion proteins, systems, or methods described herein is a meganuclease. Meganucleases generally refer to rare-cutting endonucleases or homing endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A meganuclease can generate a double-stranded break. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-MsoI meganuclease, I-SceI meganuclease, variants thereof, derivatives thereof, and fragments thereof. Detailed descriptions of useful meganucleases and their application in gene editing are found, e.g., in Silva et al., Curr Gene Ther, 2011, 11 (1): 11-27; Zaslavoskiy et al., BMC Bioinformatics, 2014, 15:191; Takeuchi et al., Proc Natl Acad Sci USA, 2014, 111 (11): 4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the nuclease domain of a meganuclease comprises a modified form of a wild-type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

### B. Nonspecific End-Processing Enzyme

The fusion proteins provided herein comprise a nonspecific end-processing enzyme. Nonspecific end-processing enzymes are polypeptides that modify a terminal end of a polynucleotide in a non-sequence specific manner. In some embodiments, the nonspecific end-processing enzyme is a nonspecific exonuclease. As used herein, the term “exonuclease” refers to an enzyme that cleaves a polynucleotide from the 5′ or 3′ end. A 5′ to 3′ exonuclease cleaves a polynucleotide exclusively in the 5′ to 3′ direction. A 3′ to 5′ exonuclease cleaves a polynucleotide exclusively in the 3′ to 5′ direction. A bi-directional exonuclease can cleave polynucleotides in either direction. Suitable exonucleases are described in, e.g., Lovett, 2011, ASM Journals EcoSal Plus 4 (2): 10.1128/ecosalplus.4.4.7 and Shevelev and Hübscher, 2002, Nature Reviews Molecular Cell Biology 3:364-376.

In some embodiments, the nonspecific exonuclease is T5Exo, Trex2 (a non-processive, 3′-to-5′ exonuclease that functions as a homodimer), E. coli exonuclease I, exonuclease III, exonuclease T, exonuclease IX, Exonuclease X, RecJ, Pol II, Pol III ε; WRN, MRE11, APE1, VDJP, RAD1, RAD9, p53, or Trex1.

In some embodiments, the nonspecific end-processing enzyme comprises an amino acid sequence having at least 70% (e.g., at least 75%, at least 80%, at least 85%, at least 90%, at least 91%, at least 92%, at least 93%, at least 94%, at least 95%, at least 96%, at least 97%, at least 98%, at least 99%, or 100%) identity to any one of SEQ ID NOs: 4, 5, 18, 19, 20, 22, or 58-74.

### C. Dimerization

Dimerization refers to when two noncovalently-linked protein domains come together to function as a single unit (i.e., a “dimer”). A non-limiting list of types of dimers includes homodimer, heterodimer, oligomerization/multimerization, autonomous dimerization, and inducible dimerization. See, e.g., US2016/0024485; US2020/0199254; WO1999/010510; WO2022/040909; and US2018/163195. In some embodiments, the fusion proteins provided herein form dimers (e.g., homodimers). In some embodiments, this is through protein-protein interactions of the non-specific end-processing enzymes. In some embodiments, the nonspecific end-processing enzyme is capable of dimerizing. In some instances, the nonspecific end-processing enzyme is a monomer of a protein that dimerizes. In some instances, the nonspecific end-processing enzyme comprises a dimerization domain. In some embodiments, fusion proteins comprising a nonspecific end-processing enzyme that is capable of dimerizing will form fusion protein dimers (or complexes with more than two monomers) via dimerization of the nonspecific end-processing enzyme.

In some embodiments, the nonspecific end-processing enzyme is able to dimerize in its endogenous form. As an example, Trex2 is able to dimerize, and is known to function as a homodimer. In some embodiments, the nonspecific end-processing enzyme comprises a domain that contributes to dimerization. In some embodiments, the nonspecific end-processing enzyme is able to dimerize autonomously.

In some embodiments, the nonspecific end-processing enzyme is an engineered polypeptide that has gained dimerization function, e.g., through addition of a dimerization domain. There are a wide variety of protein dimerization domains known in the art, including, for example, antibody Fc domains and commercially available dimerization systems (e.g., iDimerize® system, Takara Bio USA) In some embodiments, dimerization is achieved through use of any of the polypeptide interaction strategies described below in Section III.D. In some instances, the dimerization domain can be positioned at the N′ terminal or C′ terminal end of the nonspecific end-processing enzyme.

### D. Additional Fusion Protein Domains

In some embodiments, the fusion proteins provided herein comprise one or more linkers. Linkers, also referred to as spacers, as used herein are flexible molecules or a flexible stretch of molecules that joins or connects two portions (e.g., domains) of a fusion protein or a modified protein as provided herein. In some embodiments, the linker is a polypeptide. Proteins with domains joined by polypeptide linkers are referred to as fusion proteins. In some embodiments, the linker is a non-peptide linker. Proteins with domains joined by polypeptide linkers are referred to as modified proteins. It will be understood that, where fusion proteins are discussed throughout the present disclosure, modified proteins are generally also contemplated, where feasible.

The linker may increase the range of orientations that may be adopted by the domains of the fusion protein or modified protein. The linker may be optimized to produce desired effects in the fusion protein or modified protein. Aspects of linker design and considerations are described, for example, in Chen, X. et al., Adv Drug Deliv Rev. 2013 Oct. 15; 65 (10): 1357-1369, and Klein, J. S. et al. 2014 Protein Eng. Des. Sel. 27 (10): 325-330. In some embodiments, the proteins provided herein comprise a peptide linker. In some embodiments, the proteins provided herein comprise a non-peptide linker. In some embodiments, the proteins provided herein comprise a peptide linker and a non-peptide linker. The proteins provided herein may also comprise a plurality of linkers, including at least one peptide linker, at least one non-peptide linker, or at least one peptide linker and at least one non-peptide linker.

Linkers may be short or long, flexible or rigid. See, e.g., PCT/US2020/051383 incorporated herein by reference in its entirety, and WO 2020/168102, incorporated herein by reference in its entirety, and US 2021/0017506, incorporated herein by reference in its entirety.

In some embodiments, the length of a linker may affect one or more functions of the fusion protein. Selection of linkers to achieve the desired length is within the ability of one skilled in the art. In some embodiments, a peptide linker may be, for example, 5 to 100 or more amino acids in length (e.g., 5 aa, 10 aa, 15 aa, 20 aa, 25 aa, 30 aa, 35 aa, 40 aa, 45 aa, 50 aa, 55 aa, 60 aa, 65 aa, 70 aa, 75 aa, 80 aa, 85 aa, 90 aa, 95 aa, or 100 aa).

Depending on length, linker sequence may have various conformations in secondary structure, such as helical, B-strand, coil/bend, and turns. In some instances, a linker sequence may have an extended conformation and function as an independent domain that does not interact with the adjacent protein domains. Linker sequences may be flexible or rigid. Flexible linkers provide a certain degree of movement or interaction between the polypeptide domains and are generally rich in small or polar amino acids such as Gly and Ser (e.g., at least 90%, at least 95%, at least 98%, at least 99%, or all of the amino acid residues of the linker are either Gly or Ser). A rigid linker can be used to keep a fixed distance between the domains and to help maintain their independent functions. Linker attachment can be through an amide linkage (e.g., a peptide bond) or other functionalities as discussed further below.

In some embodiments, a peptide linker described herein comprises an amino acid sequence with at least 90% sequence identity to SEQ ID NO:7. In some embodiments, the linker comprises one or more repeats (e.g., 2 repeats, 3 repeats, 4 repeats, 5 repeats 6 repeats, or more) of GGGGS (SEQ ID NO: 125) and/or one or more repeats of GSSGSS (SEQ ID NO: 126). Additional exemplary peptide linkers include, but are not limited to, peptide linkers comprising SGSETPGTSESATPE (SEQ ID NO:127), SGSETPGTSESATPES (SEQ ID NO: 128), (GGGGS) 3 (SEQ ID NO:129), (GGGGS) 5 (SEQ ID NO:130), (GGGGS) 10 (SEQ ID NO: 131), GGGGGGGG (SEQ ID NO:132), GSAGSAAGSGEF (SEQ ID NO:133), A(EAAAK) 3A (SEQ ID NO:134), or A(EAAAK) 10A (SEQ ID NO:135). Additional non-limiting exemplary linkers that can be used include those disclosed in PCT/US2020/051383, Chen et al., Adv. Drug. Deliv. Rev. 65 (10): 1357-1369 (2014) and Rosemalen et al., Biochemistry 2017, 56, 50, 6565-6574, the entire contents of both of which are herein incorporated by reference.

In some embodiments, a non-peptide linker can comprise any of a number of known chemical linkers. Exemplary chemical linkers can include one or more units of beta-alanine, 4-aminobutyric acid (GABA), (2-aminoethoxy) acetic acid (AEA), 5-aminobexanoic acid (Ahx), PEG multimers, and trioxatricdeacan-succinamic acid (Ttds). In some embodiments, the non-peptide linker comprises one or more units of polyethylene glycol (PEG), which is commonly used as a linker for conjugation of polypeptide domains due to its water solubility, lack of toxicity, low immunogenicity, and well-defined chain lengths. See, e.g., Ramirez-Paz, J., et al., PLOS One 13 (7): e0197643 (2018). The number of PEG linkage units may be selected based on the desired length of the linker.

Modified proteins comprising a non-peptide linker can be produced in a variety of ways. For example, a site-directed nuclease and a nonspecific end-processing enzyme may be produced separately (e.g., in vitro or by expression in and purification from host cells) and chemically linked in vitro. In some embodiments, a site-directed nuclease, a nonspecific end-processing enzyme, and a linker can each be produced separately and chemically linked in vitro. Various chemical linkers may be used to cross link two amino acid residues.

Also contemplated herein are embodiments in which the site-directed nuclease and the nonspecific end-processing enzyme as described above are used separately (e.g., introduced into cells separately or applied to target nucleic acids separately) and brought into proximity to form a complex without using linkers as described above. Various methods of forming complexes between two or more polypeptides are known in the art and include, but are not limited to, using protein-protein interaction strategies (e.g., SunTag, coiled-coil, etc.), using RNA-aptamers and associated binding proteins (e.g., MS2, N22, etc.), and Tag: Catcher strategies. For example, a site-directed nuclease of the present disclosure may comprise an MS2 RNA aptamer, which would facilitate interaction with a nonspecific end-processing enzyme comprising an MS2 coat protein.

In some embodiments, the fusion proteins provided herein comprise a targeting sequence which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a nuclease) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof. In some embodiments, the fusion protein comprises a nuclear localization signal. Non-limiting examples of NLSs include an NLS sequence derived from: the NLS of the SV40 virus large T-antigen, having the amino acid sequence PKKKRKV (SEQ ID NO:8); the NLS from nucleoplasmin (e.g. the nucleoplasmin bipartite NLS with the sequence KRPAATKKAGQAKKKK (SEQ ID NO: 136)); the c-myc NLS having the amino acid sequence PAAKRVKLD (SEQ ID NO:137) or RQRRNELKRSP (SEQ ID NO:138); the hRNPA1 M9 NLS having the sequence NQSSNFGPMKGGNFGGRSSGPYGGGGQYFAKPRNQGGY (SEQ ID NO:139); the sequence RMRIZFKNKGKDTAELRRRRVEVSVELRKAKKDEQILKRRNV (SEQ ID NO: 140) of the IBB domain from importin-alpha; the sequences VSRKRPRP (SEQ ID NO: 141) and PPKKARED (SEQ ID NO:142) of the myoma T protein; the sequence PQPKKKPL (SEQ ID NO: 143) of human p53; the sequence SALIKKKKKMAP (SEQ ID NO: 144) of mouse c-abl IV; the sequences DRLRR (SEQ ID NO: 145) and PKQKKRK (SEQ ID NO: 146) of the influenza virus NS1; the sequence RKLKKKIKKL (SEQ ID NO:147) of the Hepatitis virus delta antigen; the sequence REKKKFLKRR (SEQ ID NO:148) of the mouse Mx1 protein; the sequence KRKGDEVDGVDEVAKKKSKK (SEQ ID NO:149) of the human poly(ADP-ribose) polymerase; the sequence RKCLQAGMNLEARKTKK (SEQ ID NO: 150) of the steroid hormone receptors (human) glucocorticoid; and the sequence KRPRDRHDGELGGRKRAR (SEQ ID NO:151) of the Agrobacterium VirD2 protein.

In some embodiments, the fusion protein provided herein comprises an amino acid sequence having at least 70% (e.g., at least 75%, at least 80%, at least 85%, at least 90%, at least 91%, at least 92%, at least 93%, at least 94%, at least 95%, at least 96%, at least 97%, at least 98%, at least 99%, or 100%) identity to any one of SEQ ID NOs: 50-57.

Any of the polypeptides and fusion proteins described herein can further comprise a detectable moiety, for example, a fluorescent protein or fragment thereof. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90 (3): 1103-1163 (2010); and Specht et al., “A Critical and Comparative Review of Fluorescent Tools for Live-Cell Imaging,” Annual Review of Physiology 79:93-117 (2017)).

Any of the polypeptides described herein can further comprise an affinity tag, for example, a polyhistidine tag (e.g., (His)₆ (SEQ ID NO:152)), an HA tag (e.g., YPYDVPDYA (SEQ ID NO: 153)), albumin-binding protein, alkaline phosphatase, an AU1 epitope, an AU5 epitope, a biotin-carboxy carrier protein (BCCP), a FLAG epitope (e.g., DYKDDDDK (SEQ ID NO: 154), or a MYC epitope (e.g., EQKLISEEDL (SEQ ID NO:155)), to name a few. See, Kimple et al. “Overview of Affinity Tags for Protein Purification, Curr. Protoc. Protein Sci. 73: Unit-9.9 (2013).

### E. Variants

Also provided herein are variants of the polypeptides of this disclosure. Polypeptide variants retain their respective biological activity, unless explicitly noted otherwise. For example, variants of a site-directed nuclease polypeptide retain the biological function of the full length, native sequence site directed nuclease. In another example, variants of the nonspecific end-processing enzyme retain the biological function of the full length, native sequence nonspecific end-processing enzyme.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-587 (2013); Xie et la. “Adding amino acids to the genetic repertoire,” 9 (6): 548-54 (2005)); and all references cited therein. B and γ amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

### IV. Recombinant Nucleic Acids, Constructs, Vectors, and Host Cells

Also provided herein are recombinant nucleic acids encoding any of the polypeptides described herein. For example, a recombinant nucleic acid encoding a polypeptide that has at least 70% (e.g., at least 75%, at least 80%, at least 85%, at least 90%, at least 91%, at least 92%, at least 93%, at least 94%, at least 95%, at least 96%, at least 97%, at least 98%, at least 99%, or 100%) identity to any of SEQ ID NOs: 4-6, 18-20, 22, or 50-74 is also provided. Also provided are recombinant nucleic acids having at least 70% identity to any of SEQ ID NOs: 21, 32, or 33.

Also provided is a DNA construct comprising a promoter operably linked to a recombinant nucleic acid encoding a fusion protein or domains thereof as described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription.

The term “promoter” as used herein refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped) and is capable of functioning even when moved either upstream or downstream from the promoter. The meaning of the term “promoter” includes “promoter regulatory sequences.”

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters”, if the promoters direct RNA synthesis preferentially in certain tissues (RNA synthesis may occur in other tissues at reduced levels). Since patterns of expression of a chimeric gene (or genes) introduced into a plant are controlled using promoters, there is an ongoing interest in the isolation of novel promoters that are capable of controlling the expression of a chimeric gene (or genes) at certain levels in specific tissue types or at specific plant developmental stages.

Certain promoters are able to direct RNA synthesis at relatively similar levels across all tissues of a plant. These are called “constitutive promoters” or “tissue-independent” promoters. Constitutive promoters can be divided into strong, moderate, and weak categories according to their effectiveness to directing RNA synthesis. Since it is necessary in many cases to simultaneously express a chimeric gene (or genes) in different tissues of a plant to get the desired functions of the gene (or genes), constitutive promoters are especially useful in this regard. Though many constitutive promoters have been discovered from plants and plant viruses and characterized, there is still an ongoing interest in the isolation of more novel constitutive promoters, synthetic or native, which are capable of controlling the expression of a chimeric gene (or genes) at different levels and the expression of multiple genes in the same transgenic plant for gene stacking.

Among the most commonly used promoters are the nopaline synthase (NOS) promoter (Ebert et al., Proc. Natl. Acad. Sci. USA 84:5745-5749 (1987)); the octapine synthase (OCS) promoter; caulimovirus promoters such as the cauliflower mosaic virus (CaMV) 19S promoter (Lawton et al., Plant Mol. Biol. 9:315-324 (1987)); the light inducible promoter from the small subunit of rubisco (Pellegrineschi et al., Biochem. Soc. Trans. 23 (2): 247-250 (1995)); the Adh promoter (Walker et al., Proc. Natl. Acad. Sci. USA 84:6624-66280 (1987)); the sucrose synthase promoter (Yang et al., Proc. Natl. Acad. Sci. USA 87:414-44148 (1990)); the R gene complex promoter (Chandler et al., Plant Cell 1:1175-1183 (1989)); the chlorophyll a/b binding protein gene promoter; and the like.”

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters. Promoters which are useful for plant transgene expression include those that are inducible, viral, synthetic, constitutive (Odell Nature 313:810-812 (1985)), temporally regulated, spatially regulated, tissue specific, and spatial temporally regulated. Using the regulatory elements described herein, numerous agronomic genes can be expressed in transformed plants. More particularly, plants can be genetically engineered to express various phenotypes of agronomic interest.”

In some embodiments of the DNA constructs provided herein, the promoter can be a eukaryotic or a prokaryotic promoter. In some embodiments, the promoter is an inducible promoter, a native inducible promoter (e.g., drought-inducible Rab17), a synthetic inducible promoter (e.g., auxin-inducible DR5, estradiol-inducible XVE/pLex, dexamethasone-inducible GVG/Gal4), a constitutive promoter (e.g., ZmUbq1, OsAct1, OsTub3, EF), an egg cell-specific promoter (e.g., EC1, EC2, EC3, EC4, EC5), a pollen-specific promoter, an apical meristem tissue-specific promoter, or a promoter with enriched expression in the zygote. In some embodiments, the promoter is a floral mosaic promoter (e.g., ZmBde1, OsAP1). In some embodiments, the promoter is a ubiquitin 4 promoter, an actin promoter, a tubulin promoter, a MADS box promoter, or a plant virus promoter. Suitable promoters are disclosed, e.g., in U.S. Pat. No. 10,519,456, the entire content of which is herein incorporated by reference, and PCT/US2022/020690, incorporated herein by reference.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of a fusion protein. The cassette may additionally contain at least one additional gene or genetic element to be cotransformed into the cell or organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence (i.e., a nucleic acid sequence that is transcribed into RNA such as mRNA, IRNA, tRNA, snRNA, ncRNA, lncRNA, sense RNA, or antisense RNA, regardless of whether the RNA is then translated to produce a protein) in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be used.

Further provided is a vector comprising a recombinant nucleic acid or DNA construct set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. See generally, Sambrook et al. Molecular Cloning: A Laboratory Manual, 4th ed., Cold Spring Harbor Laboratory Press, Cold Spring Harbor, 2012. The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, geneticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, HEK-293 cells, HEK-293T cells, U2OS cells, or any other primary or transformed cell line. Other suitable host cell lines include COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

Also provided herein are host cells comprising the recombinant nucleic acids, DNA constructs, and/or vectors described herein as well as methods of making such cells. In some embodiments, the cell is a plant cell. In some embodiments, the plant cell is a maize plant cell, a soybean plant cell, a rice plant cell, a wheat plant cell, or a sunflower plant cell.

A host cell comprising a nucleic acid or a vector described herein is provided. The host cell can be an in vitro, ex vivo, or in vivo host cell. Host cells as provided herein are capable of expressing the fusion protein. Cell populations of any of the host cells described herein are also provided. In some embodiments, the cell population comprises a plurality of cells, wherein the plurality of cells comprise a recombinant nucleic acid encoding the fusion protein as described herein. In some embodiments, the cell population comprises a plurality of cells, wherein the plurality of cells comprises a DNA construct encoding the fusion protein as described herein. In some embodiments, the cell population comprises a plurality of cells, wherein the plurality of cells comprises a vector comprising a recombinant nucleic acid or a DNA construct encoding the fusion protein as described herein. In some embodiments, the cell population comprises a plurality of cells, wherein the plurality of cells comprise a plurality of any of the host cells described herein. In some embodiments, a plurality of cells of any of the cell populations described herein express a fusion protein as described herein.

In some embodiments, the provided cells express the fusion protein stably or transiently. Stable expression of the fusion protein in a cell refers to integration of any of the nucleic acids, DNA constructs, or vectors described herein into the genome of the cell, thereby allowing the cell to express the fusion protein. Transient expression refers to expression of the fusion protein directly from any of the nucleic acids, DNA constructs, and/or vectors following introduction into the cell (i.e., the gene encoding the fusion protein is not integrated into the genome of the cell).

In some embodiments, the provided cells express the fusion protein constitutively or inducibly. Constitutive expression refers to ongoing, continuous expression of a gene (i.e., of a protein), whereas inducible expression refers to gene (protein) expression that is responsive to a stimulus. Inducible expression is generally regulated via an inducible promoter, a description of which is included above.

A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, supra; Ausubel, ed. (1995) Current Protocols in Molecular Biology, John Wiley & Sons, as well as Freshney (1994) Culture of Animal Cells, a Manual of Basic Technique, 3^{rd} Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be a HEK-293T cell, a HEK-293 cell, a Chinese hamster ovary (CHO) cell, a U20S cell, or any other primary or transformed cell. In some embodiments, the cell can be a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

As used herein, the phrase “introducing” in the context of introducing a nucleic acid into a cell (e.g., a prokaryotic cell, a bacterial cell, a eukaryotic cell, a plant cell) refers to the translocation of the nucleic acid sequence from outside a cell to inside the cell. In some cases, introducing refers to translocation of the nucleic acid from outside the cell to inside the nucleus of the cell. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acie constructs. Accordingly, such polynucleotides can be introduced into cells (e.g., plant cells) in a single transformation event, in separate transformation events, or, e.g., as part of a breeding protocol. Various methods of introducing a nucleic acid into a cell are contemplated, including but not limited to, electroporation, nanoparticle delivery, biolistic transformation, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease, a transcription activator-like effector nuclease (TALEN), a zinc finger nuclease (ZFN), or a megaTAL (MT) can also be used to introduce a nucleic acid, for example, a nucleic acid encoding a fusion protein described herein, into a host cell. See Li et al. Signal Transduction and Targeted Therapy 5, Article No. 1 (2020).

Transformation of a cell may be stable or transient. Thus, a transgenic cell, plant cell, plant and/or plant part of the invention can be stably transformed or transiently transformed. Transformation can refer to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g. via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue. As used herein, the phrase “biolistic transformation” refers to a method of introducing RNA or DNA into cells (e.g., plant cells) directly, in which RNA or DNA is mixed with heavy metal particles (e.g., tungsten or gold) and released into the cell (e.g., the plant cell) using high speed pressure to allow the RNA or DNA to penetrate the cell (e.g., to penetrate the plant cell wall).

The CRISPR/Cas system can also be used to edit the genome of a host cell or organism. As detailed above, the “CRISPR/Cas” system refers to a widespread class of bacterial systems for defense against foreign nucleic acid. Any of the CRISPR/Cas system components described herein may be used to introduce fusion proteins, recombinant nucleic acids, or systems into the genome of a host cell or organism. Methods for CRISPR/Cas system mediated genome editing are known in the art. It will be understood that use of a CRISPR/Cas system for introduction of fusion proteins, recombinant nucleic acids, or systems described herein into the genome of a host cell or organism is different from the particular methods and systems provided herein.

Any of the fusion proteins described herein can be purified or isolated from a host cell or population of host cells. For example, a recombinant nucleic acid encoding any of the fusion proteins described herein can be introduced into a host cell under conditions that allow expression of the fusion protein. In some embodiments, the recombinant nucleic acid is codon-optimized for expression. After expression in the host cell, the fusion protein can be isolated or purified using purification methods known in the art.

### V. Systems

In another aspect, provided herein are systems useful for editing one or more nucleic acids. The systems comprise one or more of the fusion proteins (or recombinant nucleic acids, constructs, vectors, or host cells) described above. In some embodiments, the systems further comprise one or more additional elements that are useful for editing one or more nucleic acids. For example, a system provided herein can further comprise a donor polynucleotide. As another example, a system comprising a fusion protein comprising a Cas nuclease may further comprise one or more guide nucleic acids and/or one or more donor polynucleotide sequences. Donor polynucleotides and guide nucleic acids are detailed below. The systems provided herein are useful for performing the methods described in Section VI of this disclosure.

A. Donor polynucleotides

The systems and methods of the present disclosure may comprise a donor polynucleotide. A “donor polynucleotide”, “donor molecule”, or “donor template” is a nucleotide polymer or oligomer intended for insertion at a target polynucleotide, typically a target genomic site. The donor sequence may be one or more transgenes, expression cassettes, or nucleotide sequences of interest. A donor molecule may be a donor DNA molecule, either single stranded, partially double-stranded, or double-stranded. The donor polynucleotide may be a natural or a modified polynucleotide, a RNA-DNA chimera, or a DNA fragment, either single- or at least partially double-stranded, or a fully double-stranded DNA molecule, or a PGR amplified ssDNA or at least partially dsDNA fragment. In some embodiments, the donor DNA molecule is part of a circularized DNA molecule. In some instances, a fully double-stranded donor DNA can provide increased stability as dsDNA fragments are generally more resistant than ssDNA to nuclease degradation.

The donor molecule may comprise at least 10 contiguous nucleotides (often referred to as a homology arm), wherein the nucleic acid molecule is at least 70% identical to a genomic nucleotide sequence, such that these contiguous nucleotides are sufficient for homologous recombination of the donor DNA molecule into the genome of the cell at the targeted genomic DNA sequence following cleavage, e.g., by a site-directed nuclease. In some embodiments, the donor DNA molecule can comprise at least about 10, 20, 30, 50, 70, 80, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or 20,000 nucleotides, including any value within this range not explicitly recited herein, wherein the donor DNA molecule is at least 70%, 75%, 80%, 85%, 90%, 91%, 92%, 93%, 94%, 95%, 96%, 97%, 98%, 99%, or 100% identical to a genomic nucleic acid sequence. In some embodiments, the donor DNA molecule may be substantially complementary to a genomic nucleic acid sequence. In some embodiments, the donor DNA molecule comprises heterologous nucleic acid sequence. In some embodiments, the donor DNA molecule comprises at least one expression cassette. In some embodiments, the donor DNA molecule may comprise a transgene, which comprises at least one expression cassette. In some embodiments, the donor DNA molecule comprises an allelic modification of a gene which is native to the target genome. The allelic modification can comprise at least one nucleotide insertion, at least one nucleotide deletion, and/or at least one nucleotide substitution. In some embodiments, the allelic modification can comprise a small insertion or deletion. In some embodiments, the donor DNA molecule comprises homologous arms to the target genomic site. In some embodiments, the donor DNA molecule comprises at least 100 contiguous nucleotides at least 90% identical to a genomic nucleic acid sequence, and optionally may further comprise a heterologous nucleic acid sequence such as a transgene.

The donor polynucleotide may be any suitable nucleic acid. In some embodiments, the donor nucleic acid is a portion of a donor template. In some embodiments, the donor template is part of a plasmid or linear nucleic acid. In some embodiments, the donor nucleic acid is a portion of a chromosome.

In some embodiments, the donor polynucleotide comprises a nucleotide sequence having at least 70% (e.g., at least 75%, at least 80%, at least 85%, at least 90%, at least 91%, at least 92%, at least 93%, at least 94%, at least 95%, at least 96%, at least 97%, at least 98%, at least 99%, or 100%) identity to SEQ ID NO:75 or SEQ ID NO:76.

### B. Guide Nucleic Acids

In some cases, the systems and methods described herein comprise at least one guide nucleic acid polynucleotide. In some cases, the systems and methods described herein comprise a plurality of guide nucleic acids. In some embodiments, the polynucleotide can be deoxyribonucleic acid (DNA). In some cases, the DNA sequence can be single-stranded or doubled-stranded. In some embodiments, the at least one guide nucleic acid polynucleotide can be ribonucleic acid (guide RNA).

In some embodiments, the nuclease can be complexed with the at least one guide RNA polynucleotide. The at least one guide RNA polynucleotide can comprise a nucleic-acid targeting region that comprises a complementary sequence to a nucleic acid sequence on the targeted polynucleotide such as the targeted genomic loci or genes to confer sequence specificity of nuclease targeting. In some embodiments, the at least one guide RNA polynucleotide can comprise two separate nucleic acid molecules, which can be referred to as a double guide nucleic acid or a single nucleic acid molecule, which can be referred to as a single guide nucleic acid (e.g., single guide RNA or sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracrRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

Whether a nuclease requires a crRNA molecule only or whether it requires both a crRNA molecule and a tracrRNA molecule (whether covalently linked or not) depends on the CRISPR-associated nuclease used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a protospacer-adjacent motif (PAM) within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., a spacer sequence of the at least one guide polynucleotide as disclosed herein) and the target nucleic acid (e.g., a protospacer sequence of the one or more target loci as disclosed herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

In some embodiments, the dsRNA duplex of the Cas protein-binding segment can have a length of 36 base pairs. The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., the sequence that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

Guide nucleic acids of the systems of the disclosure can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence; a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleotide can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can commonly be referred to as forming the internucleoside backbone of the guide nucleic acid. The linkage or backbone of the guide nucleic acid can be a 3′ to 5′ phosphodiester linkage.

A guide nucleic acid can comprise a modified backbone and/or modified internucleoside linkages. Modified backbones can include those that retain a phosphorus atom in the backbone and those that do not have a phosphorus atom in the backbone.

Suitable modified guide nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (such as a single inverted nucleoside residue in which the nucleobase is missing or has a hydroxyl group in place thereof). Various salts (e.g., potassium chloride or sodium chloride), mixed salts, and free acid forms can also be included.

A guide nucleic acid can comprise one or more phosphorothioate and/or heteroatom internucleoside linkages, in particular-CH2-NH—O-CH2-, —CH2-N(CH3)-O-CH2-(a methylene (methylimino) or MMI backbone),—CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-).

A guide nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage replaces a phosphodiester linkage.

A guide nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A guide nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A guide nucleic acid can comprise linked morpholino units (morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be non-ionic mimics of guide nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A guide nucleic acid can comprise one or more substituted sugar moieties. Suitable polynucleotides can comprise a sugar substituent group selected from: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C10 alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂) ₙCH₃)₂, where n and m are from 1 to about 10. A sugar substituent group can be selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH₂ CH₂OCH₃, also known as 2′-O-(2-methoxyethyl) or 2′-MOE, an alkoxyalkoxy group). A further suitable modification can include 2′-dimethylaminooxyethoxy, (a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE), 2′-O—CH₂-O—CH₂—N(CH₃)₂.

Other suitable sugar substituent groups can include methoxy (—O—CH₃), aminopropoxy (—O CH₂ CH₂NH₂), allyl (—CH₂—CH═CH₂), —O-allyl (—O—CH₂—CH═CH₂) and fluoro (F). 2′-sugar substituent groups can be in the arabino (up) position or ribo (down) position. A suitable 2′-arabino modification is 2′-F. Similar modifications can also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds can also have sugar mimetics such as cyclobutyl moieties in place of the pentofuranosyl sugar.

A guide nucleic acid can also include nucleobase (or “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido (5,4-(b) (1,4)benzoxazin-2 (3H)-one), carbazole cytidine (2H-pyrimido (4,5-b) indol-2-one), pyridoindole cytidine (H-pyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid can comprise chemically linking to the guide nucleic acid one or more moieties or conjugates that can enhance the activity, cellular distribution or cellular uptake of the guide nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

In some embodiments, the at least one guide RNA polynucleotide of a system or method provided herein can bind to at least a portion of a genome (e.g., a plant genome) or a gene (e.g., a plant gene). In some cases, the at least one guide RNA polynucleotide is capable of forming a complex with a site-directed nuclease to direct the site-directed nuclease to target the portion of a target nucleic acid (e.g., a site in a genome or a gene).

In some embodiments, the systems described herein comprise at least one guide RNA polynucleotide that is able to form a complex with a site-directed nuclease portion of a fusion protein of the system. In some embodiments, the systems described herein comprise at least two (e.g., at least three, at least four, at least five, or at least six) different guide RNA polynucleotides that are able to form a complex with a site-directed nuclease portion of a fusion protein of the system.

In some embodiments, the guide nucleic acid comprises a nucleotide sequence having at least 70% (e.g., at least 75%, at least 80%, at least 85%, at least 90%, at least 91%, at least 92%, at least 93%, at least 94%, at least 95%, at least 96%, at least 97%, at least 98%, at least 99%, or 100%) identity to any one of SEQ ID NOs: 2, 3, 11, 12, 23-26, or 28-31.

Also provided herein are kits that include the components of the systems described in this disclosure. In some embodiments, the kits include one or more of the fusion proteins and/or polynucleotides described herein.

### VI. Methods

In another aspect, provided herein are methods for editing one or more nucleic acids using the fusion proteins and/or systems described herein. In some embodiments, the methods comprise contacting a nucleic acid comprising fusion protein binding sites (i.e., the nucleic acid to be edited) with at least one fusion protein as described herein, wherein contacting the nucleic acid with the at least one fusion protein results in an edit to the nucleic acid. The nucleic acid (i.e., the nucleic acid to be edited) may be any suitable nucleic acid. In some embodiments, the nucleic acid is a portion of a chromosome. In some embodiments, the nucleic acid is a portion of a genome (e.g., a plant genome).

As described herein and demonstrated in the Examples below, the methods provided herein can result in increased frequency of one or more desired nucleic acid editing outcomes (e.g., fragment excision, fragment inversion, fragment replacement via HDR, chromosomal rearrangement). By employing fusion proteins targeted (i.e., through the site-directed nuclease) to specific nucleic acid sequences (e.g., genomic sites, donor template sites), one can use the methods herein to increase (or decrease) the frequency of one or more desired nucleic acid editing outcomes. In some embodiments, the fusion proteins are targeted to a specific strand of the nucleic acid. In some embodiments, the fusion proteins are targeted to a site upstream or downstream of a nuclease cleavage site. In some embodiments, the fusion proteins are targeted to bind to a nucleic acid with a particular orientation.

In some embodiments, the nucleic acid to be edited by the method comprises a target region. As used herein, “target region” refers to a portion of a nucleic acid that is targeted for editing. For example, a target region may be a portion of a gene that is to be edited. In some embodiments, the fusion proteins provided herein are targeted to binding sites inside and/or outside of the target region (e.g., two sites inside of the target region, one site inside and one site outside of the target region, or two sites outside of the target region), as described below. In some embodiments, each fusion protein binding site is proximal to a nuclease cleavage site. In some embodiments, the target region is flanked by nuclease cleavage sites. In some embodiments, the nucleic acid comprises a first binding site that is adjacent to the 5′ end of the target region and a second binding site that is adjacent to the 3′ end of the target region.

In some embodiments, as detailed below, the nucleic acid to be edited comprises a first binding site and a second binding site. In some embodiments, the first binding site and the second binding site are different sequences, and the method comprises providing two different fusion proteins, one to bind to the first binding site and one to bind to the second binding site. In some embodiments, the first binding site and the second binding site are the same sequence, and the method comprises providing a fusion protein that can bind to both the first binding site and the second binding site.

In some embodiments, the methods herein comprise providing a donor nucleic acid comprising a third binding site and a fourth binding site. In some embodiments, the third binding site and the fourth binding site are different sequences, and the method comprises providing two different fusion proteins, one to bind to the third binding site and one to bind to the fourth binding site. In some embodiments, the third binding site and the fourth binding site are the same sequence, and the method comprises providing a fusion protein that can bind to both the third binding site and the fourth binding site. Additionally, in some embodiments, the third and/or the fourth binding site are the same sequence as the first and/or second binding site. The first, second, third, and fourth binding sites can comprise any combination of sequences, from all four having the same sequence to all four having different sequences. In some embodiments, the nucleic acid (i.e., the nucleic acid to be edited) is a portion of a first chromosome, and the donor nucleic acid is a portion of a second chromosome. In some embodiments, the first chromosome and the second chromosome are different chromosomes. In some embodiments, the first chromosome and the second chromosome are homologous chromosomes. In some embodiments, the first chromosome and the second chromosome are non-homologous chromosomes. In some embodiments, the first chromosome and the second chromosome are the same chromosome.

In some embodiments of the methods provided herein, e.g., in the exemplary embodiments described below, the site-directed nuclease of the at least one fusion protein comprises a CRISPR-associated nuclease. In such embodiments, the method can further comprise providing guide RNAs to target the fusion proteins to the binding sites. In some embodiments, the method comprises providing at least one first guide RNA and at least one second guide RNA. In some embodiments, the at least one first guide RNA comprises a nucleotide sequence having complementarity to the first binding site of the nucleic acid to be edited. In some embodiments, the at least one second guide RNA comprises a nucleotide sequence having complementarity to the second binding site of the nucleic acid to be edited. In some embodiments, where the methods comprise providing a donor nucleic acid, the methods further comprise providing at least one third guide RNA and at least one fourth guide RNA. In some embodiments, the at least one third guide RNA comprises a nucleotide sequence having complementarity to the third binding site (i.e., on the donor nucleic acid). In some embodiments, the at least one fourth guide RNA comprises a nucleotide sequence having complementarity to the fourth binding site (i.e., on the donor nucleic acid).

In some embodiments, the frequency of desired nucleic acid editing outcomes can be increased or decreased by targeting fusion proteins to bind inside and/or outside of a target region. In some embodiments of the methods provided herein, the edit made to the nucleic acid is an excision (i.e., removal), an inversion (i.e., reversal of direction), or a replacement of at least a portion of the target region. In some embodiments, the edit is a chromosomal rearrangement. In some embodiments, the chromosomal rearrangement is a reciprocal translocation. In some embodiments, the chromosomal rearrangement is a non-reciprocal translocation. Several non-limiting exemplary embodiments of fusion protein targeting and editing outcomes that increase in frequency are discussed below with reference to the accompanying figures, followed by additional discussion of various aspects of the methods. While the exemplary embodiments comprise Cas enzymes as the fusion protein SDN, and thus include discussion of gRNA-mediated binding and PAM sequences, it will be understood that other site-directed nucleases (e.g., zinc finger nucleases, TAL-effector nucleases, meganucleases, etc.) can be used for similarly targeted binding (e.g., targeting inside and/or outside of a target region), as discussed in section III.A above.

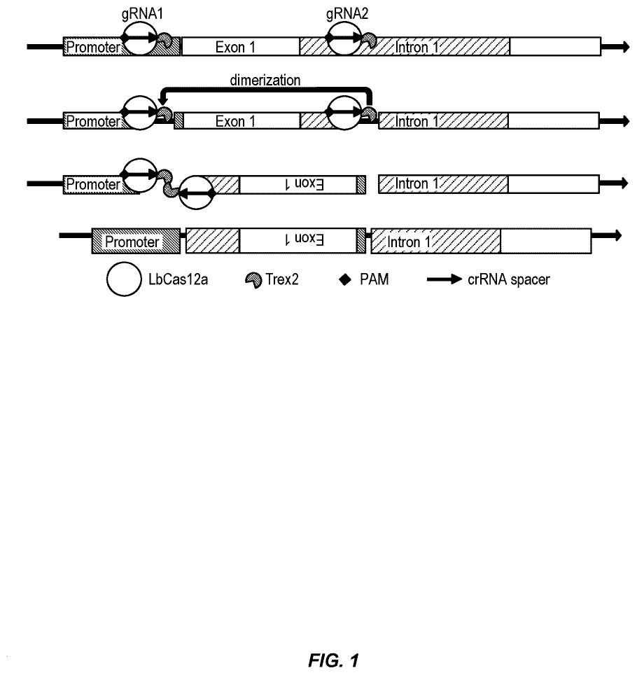

In some embodiments, as demonstrated in Example 1 of this disclosure, the methods provided herein result in an increase in the frequency of fragment inversion between paired cleavage sites on a nucleic acid (e.g., at a genomic locus). One exemplary embodiment is illustrated in FIG. 1, in which the method comprises two fusion proteins that bind to a first binding site and a second binding site on the same strand of a DNA polynucleotide. The first binding site is adjacent to the 5′ end of the target region (i.e., the region between the nuclease cleavage sites), and the second binding site is adjacent to the 3′ end of the target region. For illustrative purposes, the fusion protein in this embodiment comprises LbCas12a as the SDN linked to a Trex2 exonuclease as the nonspecific end-processing enzyme. The DNA polynucleotide is a portion of the ZmDMR6 gene, the first binding site comprises a PAM sequence located in the promoter of the gene, and the second binding site comprises a PAM sequence located in intron 1 of the gene. LbCas12a (i.e., the SDN of the fusion proteins) cleaves the polynucleotide in the 3′ direction from the PAM and the fusion proteins remain bound to the binding sites on the cleaved polynucleotide ends that comprise the PAM. Because the first binding site (indicated by “gRNA 1”) is upstream of the cleavage site (i.e., outside of the target region), the fusion protein bound to the first binding site remains bound to the cleaved polynucleotide end outside of and upstream from the target region. Because the second binding site (indicated by “gRNA 2”) is upstream of the cleavage site (i.e., inside the target region), the fusion protein bound to the second binding site remains bound to the downstream end of the target region. Without being bound by any particular theory, the dimerization of the fusion proteins (i.e., through dimerization of Trex2) bound to the first and second binding sites will bring the downstream end of the target region and the end of the polynucleotide upstream of the target region into close proximity, where they will be joined through a DNA repair mechanism such as NHEJ or MMEJ. Joining of the remaining polynucleotide ends (i.e., those that are not bound by a fusion protein) through DNA repair mechanisms thus results in an inversion of the target region.

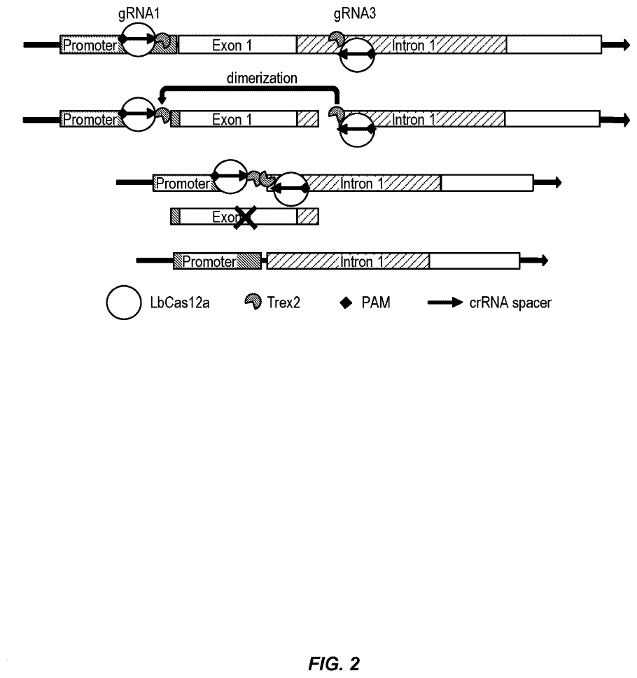

In some embodiments, as described in Example 3 of this disclosure, the methods provided herein result in an increase in the frequency of fragment excision (i.e., removal) between paired cleavage sites on a nucleic acid (e.g., at a genomic locus). One exemplary embodiment is illustrated in FIG. 2, in which the method comprises two fusion proteins that bind to a first binding site and a second binding site on opposite strands of a DNA polynucleotide. The first binding site is adjacent to the 5′ end of the target region, and the second binding site is adjacent to the 3′ end of the target region. For illustrative purposes, the fusion protein in this embodiment comprises LbCas12a as the SDN linked to a Trex2 exonuclease as the nonspecific end-processing enzyme. The DNA polynucleotide is a portion of the ZmDMR6 gene, the first binding site comprises a PAM sequence located in the promoter of the gene, and the second binding site comprises a PAM sequence located in intron 1 of the gene. LbCas12a (i.e., the SDN of the fusion proteins) cleaves the polynucleotide in the 3′ direction from the PAM and the fusion proteins remain bound to the binding sites on the cleaved polynucleotide ends that comprise the PAM. Because the first binding site (indicated by “gRNA 1”) is upstream of the cleavage site (i.e., outside of the target region), the fusion protein bound to the first binding site remains bound to the cleaved polynucleotide end outside of and upstream from the target region. Because the second binding site (indicated by “gRNA 2”) is downstream of the cleavage site (i.e., outside the target region), the fusion protein bound to the second binding site remains bound to the cleaved polynucleotide end outside of and downstream from the target region. Without being bound by any particular theory, the dimerization of the fusion proteins bound to the first and second binding sites will bring the cleaved polynucleotide ends upstream of and downstream of the target region into close proximity, where they will be joined through a DNA repair mechanism such as NHEJ or MMEJ, resulting in excision of the target region.

In some embodiments, an increase in frequency of fragment excision as described above can also be achieved by methods using fusion proteins targeted within a target region. One exemplary embodiment of such a method comprises two fusion proteins that bind to a first binding site and a second binding site on opposite strands of a DNA polynucleotide. The first binding site is adjacent to the 5′ end of the target region, the second binding site is adjacent to the 3′ end of the target region, and both the first binding site and the second binding site are within the target region. Thus, the fusion proteins remain bound to the terminal ends of the target region following SDN-mediated cleavage. Without being bound by any particular theory, the dimerization of the fusion proteins bound to the first and second binding sites will bring the cleaved polynucleotide ends of the target region into close proximity, where they will be joined through a DNA repair mechanism such as NHEJ or MMEJ (i.e., forming a circular polynucleotide). Joining of the remaining polynucleotide ends (i.e., those that are not bound by a fusion protein) through DNA repair mechanisms thus results in excision of the target region.

In some embodiments, the methods provided herein further comprise providing a donor nucleic acid comprising fusion protein binding sites. As described above, donor nucleic acids can be used along with SDNs to provide a template for homology-directed repair (HDR). In some embodiments, donor nucleic acids can also be used to provide a replacement fragment to be inserted in place of a target region. In some embodiments, donor nucleic acids can be used to promote translocations (e.g., chromosomal translocations). By using fusion proteins that target specific sequences in both the nucleic acid to be edited and the donor nucleic acid, one can use the methods herein to increase (or decrease) the frequency of one or more desired nucleic acid editing outcomes. In some embodiments, the donor nucleic acids provided in the methods herein comprise a third binding site and a fourth binding site (i.e., where the nucleic acid to be edited comprises a first binding site and a second binding site) and a donor nucleotide region. As used herein, “donor nucleotide region” refers to a portion of a donor nucleic acid that is flanked by fusion protein binding sites. Several non-limiting exemplary embodiments of methods comprising a donor nucleic acid are discussed below with reference to the accompanying figures.

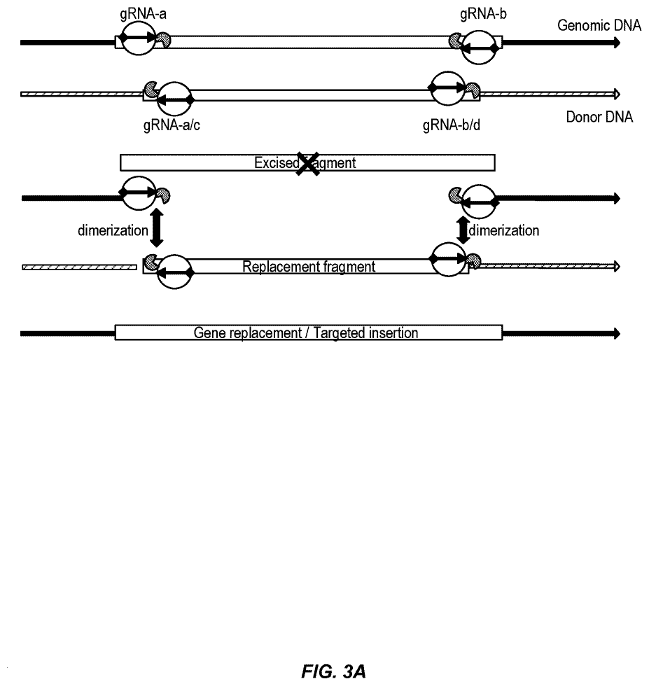

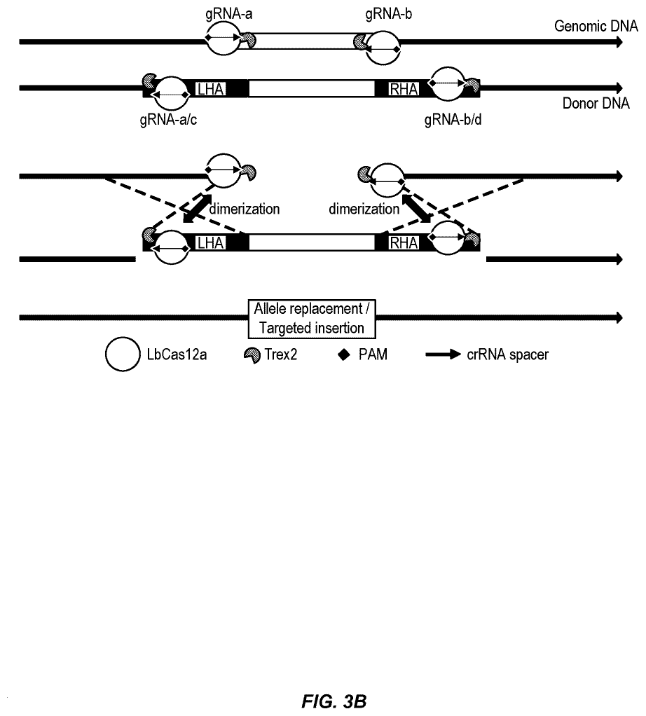

In some embodiments, as described in Example 5 of this disclosure, the methods provided herein result in an increase in the frequency of fragment replacement or targeted insertion between paired cleavage sites on a nucleic acid (e.g., at a genomic locus). Two exemplary embodiments are illustrated in FIG. 3A and FIG. 3B, in which the methods comprise two fusion proteins that bind to a first binding site and a second binding site on opposite strands of a DNA polynucleotide (i.e., the nucleotide to be edited, shown here as “Genomic DNA”) and two fusion proteins that bind to a third binding site and a fourth binding site on opposite strands of a donor nucleic acid (shown here as “Donor DNA”). In both embodiments, the first binding site is adjacent to the 5′ end of the target region (i.e., the region between the nuclease cleavage sites), the second binding site is adjacent to the 3′ end of the target region, the third binding site is adjacent to the 5′ end of the donor nucleotide region (i.e., the region of the donor nucleotide between the nuclease cleavage sites), and the fourth binding site is adjacent to the 3′ end of the donor nucleotide region. For illustrative purposes, the fusion protein in these embodiments comprises LbCas12a as the SDN linked to a Trex2 exonuclease as the nonspecific end-processing enzyme. LbCas12a (i.e., the SDN of the fusion proteins) cleaves the polynucleotides (i.e., both the nucleotide to be edited and the donor nucleic acid) in the 3′ direction from the PAM and the fusion proteins remain bound to the binding sites on the cleaved polynucleotide ends that comprise the PAM. Because the first binding site (indicated by “gRNA-a”) is upstream of the cleavage site (i.e., outside of the target region), the fusion protein bound to the first binding site remains bound to the cleaved polynucleotide end outside of and upstream from the target region. Because the second binding site (indicated by “gRNA-b”) is downstream of the cleavage site (i.e., outside the target region), the fusion protein bound to the second binding site remains bound to the cleaved polynucleotide end outside of and downstream from the target region. Because the third binding site (indicated by “gRNA-a/c”) is downstream of the cleavage site (i.e., within the donor nucleotide region), the fusion protein bound to the third binding site remains bound to the cleaved polynucleotide end of the donor nucleotide region (i.e., to the upstream end of the donor nucleotide region). Because the fourth binding site (indicated by “gRNA-b/d”) is upstream of the cleavage site (i.e., within the donor nucleotide region), the fusion protein bound to the fourth binding site remains bound to the cleaved polynucleotide end of the donor nucleotide region (i.e., to the downstream end of the donor nucleotide region). Without being bound by any particular theory, the dimerization of the fusion proteins (i.e., through dimerization of Trex2) bound to the first and third binding sites will bring the cleaved polynucleotide end upstream of the target region into close proximity with the upstream end of the donor nucleotide region, and the dimerization of the fusion proteins bound to the second and fourth binding sites will bring the cleaved polynucleotide end downstream of the target region into close proximity with the downstream end of the donor nucleotide region. Joining of the polynucleotide ends that are in close proximity through a DNA repair mechanism such as NHEJ or MMEJ will result in replacement of the target region with the donor nucleotide region. The donor nucleotide region may also be inserted in the reverse orientation (i.e., if the fusion proteins bound to the first and fourth binding sites dimerize and the fusion proteins bound to the second and third binding sites dimerize).

In the embodiment illustrated in FIG. 3A, the donor nucleotide region does not comprise homology arms, so NHEJ or MMEJ repair is more likely than HDR. In the embodiment illustrated in FIG. 3B, the donor nucleotide region comprises homology arms. Without being bound by any particular theory, in the embodiments illustrated in FIG. 3B, dimerization of the fusion proteins bound to the first binding site and the third binding site and dimerization of the fusion proteins bound to the second binding site and the fourth binding site will promote HDR-mediated repair by bringing the donor nucleotide region homology arms into close proximity with the homologous sequences in the nucleotide to be edited (e.g., the genomic DNA).

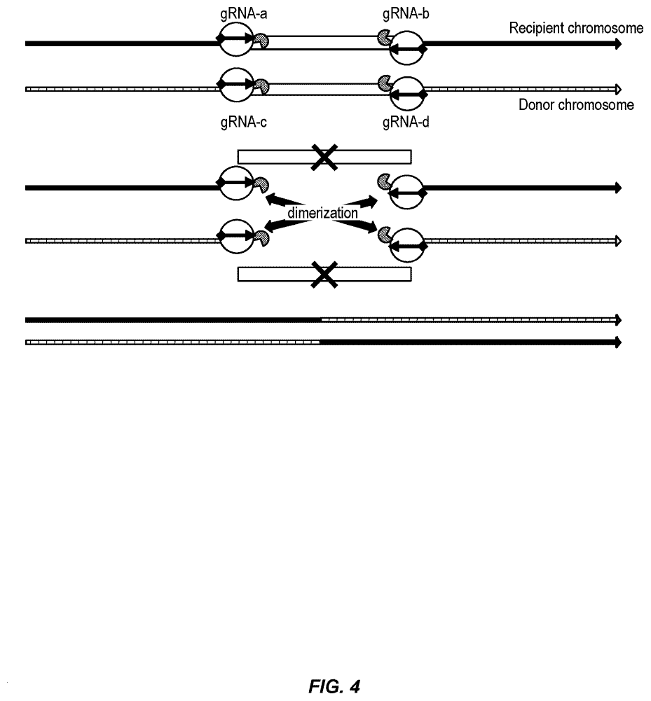

In some embodiments, as described in Example 7 of this disclosure, the methods provided herein result in an increase in the frequency of translocation between two polynucleotides (e.g., between two chromosomes). An exemplary embodiment is illustrated in FIG. 4, in which the method comprises two fusion proteins that bind to a first binding site and a second binding site on opposite strands of a DNA polynucleotide (i.e., the nucleotide to be edited, shown here as “Recipient chromosome”) and two fusion proteins that bind to a third binding site and a fourth binding site on opposite strands of a donor nucleic acid (shown here as “Donor chromosome”). The first binding site is adjacent to the 5′ end of the target region (i.e., the region between the nuclease cleavage sites), the second binding site is adjacent to the 3′ end of the target region, the third binding site is adjacent to the 5′ end of the donor nucleotide region (i.e., the region of the donor nucleotide between the nuclease cleavage sites), and the fourth binding site is adjacent to the 3′ end of the donor nucleotide region. For illustrative purposes, the fusion protein in these embodiments comprises LbCas12a as the SDN linked to a Trex2 exonuclease as the nonspecific end-processing enzyme. LbCas12a (i.e., the SDN of the fusion proteins) cleaves the polynucleotides (i.e., both the nucleotide to be edited and the donor nucleic acid) in the 3′ direction from the PAM and the fusion proteins remain bound to the binding sites on the cleaved polynucleotide ends that comprise the PAM. Because the first binding site (indicated by “gRNA-a”) is upstream of the cleavage site (i.e., outside of the target region), the fusion protein bound to the first binding site remains bound to the cleaved polynucleotide end outside of and upstream from the target region. Because the second binding site (indicated by “gRNA-b”) is downstream of the cleavage site (i.e., outside the target region), the fusion protein bound to the second binding site remains bound to the cleaved polynucleotide end outside of and downstream from the target region. Because the third binding site (indicated by “gRNA-c”) is upstream of the cleavage site (i.e., outside the donor nucleotide region), the fusion protein bound to the third binding site remains bound to the cleaved donor nucleotide end outside of and upstream from the donor nucleotide region. Because the fourth binding site (indicated by “gRNA-d”) is downstream of the cleavage site (i.e., outside the donor nucleotide region), the fusion protein bound to the fourth binding site remains bound to the cleaved donor nucleotide end outside of and downstream of the donor nucleotide region. Without being bound by any particular theory, the dimerization of the fusion proteins (i.e., through dimerization of Trex2) bound to the first and fourth binding sites will bring the cleaved polynucleotide end upstream of the target region into close proximity with the cleaved polynucleotide end downstream of the donor nucleotide region, and the dimerization of the fusion proteins bound to the second and third binding sites will bring the cleaved polynucleotide end downstream of the target region into close proximity with the cleaved polynucleotide end upstream of the donor nucleotide region. Joining of the polynucleotide ends that are in close proximity through a DNA repair mechanism such as NHEJ or MMEJ will result in a translocation of the polynucleotides (and excision of the target region and the donor nucleotide region).

Table 1 summarizes expected editing outcomes induced by the methods of the present disclosure using various fusion protein targeting strategies.

TABLE 1
Expected editing outcomes.

| | | | Exemplary |
|---|---|---|---|
| Nucleic acid to be | Donor nucleic | Expected editing | embodi- |
| edited | acid | outcome | ment |
| Binding sites on same | None | Inversion | FIG. 1 |
| strand, one within | | | and |
| target region and one | | | Example 3 |
| outside target region | | | |
| Binding sites on | None | Excision | FIG. 2 |
| opposite strand, both | | | |
| outside target region | | | |
| Binding sites on | None | Excision | Example 3 |
| opposite strand, both | | | |
| within target region | | | |
| Binding sites on | Binding sites on | Allele | FIGS. 3A |
| opposite strand, both | opposite strand, | replacement | and 3B |
| outside target region | both within donor | or targeted | |
| | nucleotide region | insertion | |
| Binding sites on | Binding sites on | Translocation | FIG. 4 |
| opposite strand, both | opposite strand, | | |
| outside target region | both outside | | |
| | donor nucleotide | | |
| | region | | |

The methods herein comprise providing at least a fusion protein and a nucleic acid to be edited, and may also comprise providing a donor nucleic acid and/or at least one guide RNA. These various components can be provided using any suitable technique. For example, providing a fusion protein can comprise introducing the fusion protein into a cell or introducing a recombinant nucleic acid, construct, or vector encoding the fusion protein into a cell. Similarly, a gRNA can be provided by introducing the gRNA itself or a nucleic acid sequence encoding the gRNA. In some embodiments, a fusion protein and a gRNA can be encoded by the same DNA construct or vector.

### EXAMPLES

### Example 1. Using LbCas12a-Trex2 Fusion to Increase the Frequency of Fragment Inversion Between Paired Guide RNA Targeting Sites in Maize Genome

This example is to demonstrate that the LbCas12a-Trex2 fusion is able to promote fragment inversion between two paired gRNA targeting sites on the same chromosome and on the same strand (e.g., as shown in FIG. 1). Maize DOWNY MILDEW RESISTANT 6 (DMR6) gene (nucleotide sequence shown in SEQ ID NO:1) was selected as an example to demonstrate this design. Similar designs could be applied to any other genomic locus in any organism.

TABLE 2
Sequences used in Example 1.

| SEQ | | | |
|---|---|---|---|
| ID | | | |
| NO. | Description | Type | Source |
| 1 | Maize DMR6 gene, full length | DNA | Zea mays |
| 2 | Guide RNA1 targeting sequence | DNA | Zea mays |
| | in ZmDMR6 promoter | | |
| 3 | Guide RNA2 targeting sequence in | DNA | Zea mays |
| | ZmDMR6 first intron | | |
| 4 | Bacteriophage T5 exonuclease | Protein | T5 phage |
| 5 | Mouse three-prime repair exonu- | Protein | Mus musculus |
| | clease 2 (Trex2) | | |
| 6 | Type V-A Cas effector from | Protein | Lachnospiraceae |
| | Lachnospiraceae | | bacterium |
| | bacterium ND2006 | | ND2006 |
| 7 | 30-a.a. GS linker | Peptide | Synthetic |
| 8 | Simian virus 40 large T-antigen | Peptide | Simian virus 40 |
| | (SV40) nuclear localization signal | | |
| 9 | Sugarcane ubiquitin 4 promoter | DNA | Saccharum |
| | | | officinarum |
| 10 | Agrobacterium nopaline | DNA | Agrobacterium |
| | synthase terminator | | tumefaciens |
| 11 | ZmDMR6 crRNA1 | RNA | Synthetic |
| 12 | ZmDMR6 crRNA2 | RNA | Synthetic |
| 13 | Rice U6 promoter | DNA | Oryza sativa |
| 14 | E. coli phosphomannose | DNA | Escherichia coli |
| | isomerase gene, coding sequence | | |
| 15 | Maize ubiquitin 1 promoter | DNA | Zea mays |
| 16 | Forward primer used for amplifying | DNA | Zea mays |
| | the genomic region edited by | | |
| | ZmDMR6 crRNA1/crRNA2 pair | | |
| 17 | Reverse primer used for amplifying | DNA | Zea mays |
| | the genomic region edited by | | |
| | ZmDMR6 crRNA1/crRNA2 pair | | |

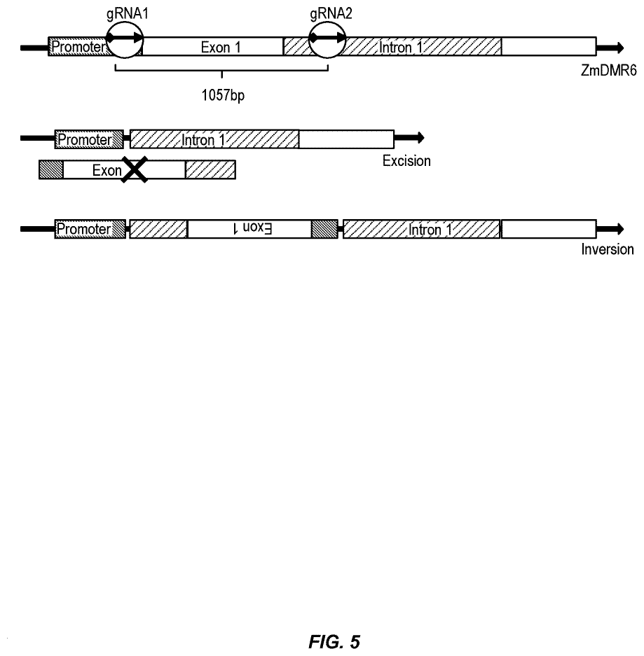

DMR6 is a well-characterized plant susceptibility gene first studied in Arabidopsis. Knockout of the gene in other plant species is expected to confer resistance against multiple pathogens. However, due to the high GC % of the DMR6 protein-coding sequence, it is difficult to find canonical TTTN PAMs that can knockout the gene through Cas12a-induced simple indel mutations. To circumvent this difficulty, one guide RNA was designed to target a sequence (SEQ ID NO:2) adjacent to a TTTG PAM in the promoter region, and a second guide RNA was designed to target a sequence (SEQ ID NO:3) adjacent to a TTTA PAM in the first intron. Co-expression of the two guide RNAs, together with the LbCas12a nuclease, should allow knocking out the gene by excising or inverting the genomic fragment between the two targeting sites, which contains the entire first exon. The guide RNA designs and expected outcomes are illustrated in FIG. 5.

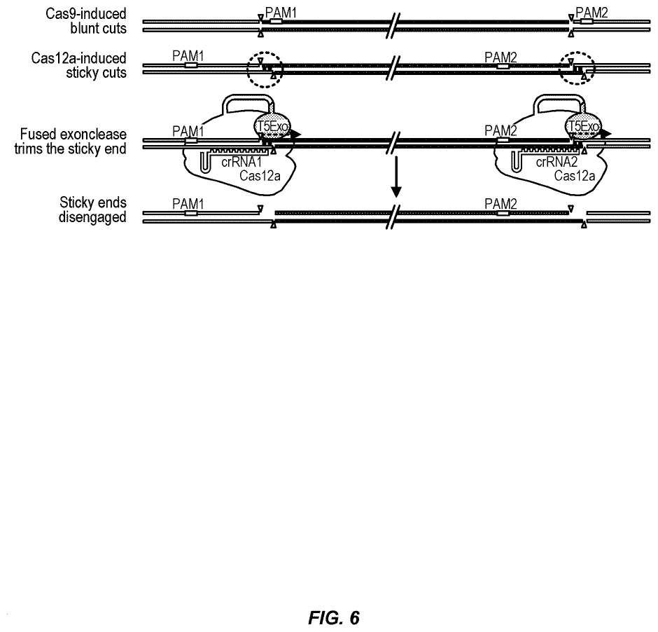

Previous internal studies had suggested compared with Cas9, it is more difficult to excise or invert the genomic sequence between paired guide RNA targeting sites using Cas12a. This could be explained by the fact that Cas12a generates double strand break with sticky ends, which could be more difficult to fall apart from each other, in contrast to the blunt ends generated by Cas9. In order to improve the frequencies of fragment excision and inversion, various DNA exonucleases including the bacteriophage T5 exonuclease (T5Exo, SEQ ID NO:4), a 5′-to-3′ exonuclease; and the mouse three-prime repair exonuclease 2 (Trex2, SEQ ID NO:5), a 3′-to-5′ exonuclease, were fused to LbCas12a (SEQ ID NO:6) through a flexible GS linker (SEQ ID NO:7), aiming to trim and disengage the sticky ends immediately after LbCas12a generates the double stand break. This hypothesis, with T5Exo as an example, is illustrated in FIG. 6.

Multiple binary vectors were constructed to test the efficacies of the Cas12a-exonuclease fusions in maize, as summarized in Table 2. The protein-coding sequence of each Cas12a-exonuclease fusions, flanked by nuclear localization signal peptides on both termini (SEQ ID NO:8), was optimized according to the maize codon usage, and was expressed by an expression cassette operably linked to a sugarcane ubiquitin 4 promoter (SEQ ID NO:9) and an Agrobacterium nopaline synthase terminator (SEQ ID NO:10). Two guide RNAs (SEQ ID NOs: 11 and 12) were expressed in a tandem array under a rice U6 promoter (SEQ ID NO: 13), and the transcript was processed into separate mature crRNAs by the intrinsic RNase activity of LbCas12a. An E. coli phosphomannose isomerase gene (SEQ ID NO: 14) operably linked to a maize ubiquitin 1 promoter (SEQ ID NO:15) and an Agrobacterium nopaline synthase terminator (SEQ ID NO:10) was served as selection marker in all vectors.

TABLE 3
DNA constructs used in Example 1.

| Construct # | D156R | Intron | Exo fusion | gRNA1 | gRNA2 | Transformation |
|---|---|---|---|---|---|---|
| 25406 | − | − | − | ZmDMR6-crRNA1 | ZmDMR6-crRNA2 | Agro LBA4404 |
| | | | | (targeting promoter, | (targeting intron1, | |
| | | | | on coding strand) | on coding strand) | |
| 26297 | + | − | − | ZmDMR6-crRNA1 | ZmDMR6-crRNA2 | Agro LBA4404 |
| 25962 | + | − | C-terminal | ZmDMR6-crRNA1 | ZmDMR6-crRNA2 | Agro LBA4404 |
| | | | Trex2 | | | |
| 25979 | + | − | C-terminal | ZmDMR6-crRNA1 | ZmDMR6-crRNA2 | Agro LBA4404 |
| | | | T5Exo | | | |
| 25961 | + | − | N-terminal | ZmDMR6-crRNA1 | ZmDMR6-crRNA2 | Biolistic |
| | | | Trex2 | | | |
| 25978 | + | − | N-terminal | ZmDMR6-crRNA1 | ZmDMR6-crRNA2 | Biolistic |
| | | | T5Exo | | | |

Each construct was delivered into callus cells derived from immature maize embryos of the Syngenta proprietary inbred line AX5707 by Agrobacterium tumefaciens-mediated transformation or biolistic transformation if the construct was not stable in Agrobacterium. The transformed calli were subject to mannose selection and plantlets were regenerated through standard tissue culture procedures. The regenerated plantlets were sampled for DNA extraction, among which the transgenic plants harboring the constructs were identified by TaqMan assays.

A PCR was designed to amplify the genomic sequence around and between the two targeting sites, and was used to characterize the editing outcomes in the transgenic plants. The expected amplicon size using wildtype template is 1,416 bp. Excision of the fragment between the targeting sites results in shorter around 350 bp. Amplicons of each transgenic plant were Sanger-sequenced to identify inverted alleles and further characterize the repaired junctions at the targeting sites.

The editing outcomes are summarized in Table 4. Compared with the control construct without a fused exonuclease domain, neither N-terminal nor C-terminal fusion of T5 exonuclease resulted in improved frequencies of desired excision or inversion. In contrast, both N-terminal and C-terminal fusions of Trex2 resulted in strikingly high inversion frequency around 20%, while moderately increased the excision frequency. Among transgenic plants harboring construct 25962 (encoding C-terminal LbCas12a-Trex2 fusion), there are three plants in which the targeted 1 kb fragment was inverted in both alleles, and two plants in which the targeted fragment was excised from one allele while that was inverted in the other allele. Full sequenced alleles resulting from editing with the C-terminal LbCas12a-Trex2 fusion, along with the unedited wild type reference sequence, are shown in Table 5, below. Most repaired junctions only lost a few additional base pairs. In Table 5, the gRNA1 target and the flanking sequences (100 bp on each side) are underlined; the gRNA2 target and the flanking sequences (100 bp on each side) are italicized. PAM sequences are in bold and protospacers are double-underlined. Sequences in lower case are reverse complement to the reference sequence. The nucleotides in square brackets are ones inserted or mutated during the repair process.

TABLE 4
Summary of editing outcomes.

| Con- | # of | % | % | | | | |
|---|---|---|---|---|---|---|---|
| struct | transgenic | Editing | Editing | 1 kb | | 1 kb | |
| # | (Cas12a+) | (gRNA1) | (gRNA2) | excision | | inversion | |
| 25406 | 84 | 67.9% | 60.7% | 3 | (3.6%) | 1 | (1.2%) |
| 26297 | 75 | 81.3% | 88.0% | 5 | (6.7%) | 1 | (1.3%) |
| 25962 | 152 | 74.3% | 82.9% | 16 | (10.5%) | 28 | (18.4%) |
| 25979 | 150 | 45.3% | 63.3% | 3 | (2.0%) | 1 | (0.7%) |
| 25961 | 119 | 82.3% | 81.5% | 13 | (10.9%) | 24 | (20.2%) |
| 25978 | 120 | 88.3% | 80.8% | 6 | (5%) | 3 | (2.5%) |

TABLE 5
C-terminal LbCas12a-Trex2 fusion editing results.

| SEQ | | |
|---|---|---|
| ID | Name | Sequence |
| 77 | WT | CTTGGATCCTGCGATCCGCCGCTGGAGCGTAACCACCGCGTCCACATGCTGCTTCATTGGTGCCGGCGCGGAGGCTG |
| | reference | CTTAGCTCGCCGCCGAGGACTACTTTGCCCGCCACGGTTACGGAGGGGTGGTGACGTCACTCACCGCCACGGCGAT |
| | | GACGCGAGGGGATATCAGTCATCAGTCCACCCAACGACCGCGGGGGGTCAACCTCTTCTCGGTCGTCGTCGCGTCG |
| | | TCACGGCTGACGCCACGGGCCCGCTGCCTGCCGGAACGCCCACGCCGAGCCACGGAGCAGTCACCACCAGACACCA |
| | | GGTTACGCGTCCGGCTTCCCGGCCGCTATAAAGAGCGCGCGACGGCGGCCCCTGGGAGAGCCATGCTAGACTGGAG |
| | | GCGGAACCGCGCACGACACCAAGCTGCCGCGCCGGACTGCTGCACGCAAGCGCAGCGCAGGACCGACCGACCTCC |
| | | GTAGGCACGCACGGCGCCGGCGGCATGGCGGAGCACCTCCTGTCGACGGCCGTGCACGACACGCTGCCGGGGAGCT |
| | | ACGTGCGGCCGGAGCCGGAGCGCCCGCGCCTCGCGGAGGTCGTGACCGGCGCGCGCATCCCCGTGGTGGACCTGGG |
| | | CAGCCCCGACCGCGGCGCGGTCGTGGCCGCCGTCGGCGACGCCTGCCGCTCGCACGGCTTCTTCCAGGTCGTCAACC |
| | | ACGGGATACACGCCGCCCTGGTCGCGGCGGTGATGGCCGCGGGGCGCGTCTTCTTCCGGCTGCCCCCCGAGGAGAA |
| | | GGCCAAGCTCTACTCCGACGACCCCGCCAGGAAGATCCGGCTGTCCACCAGCTTCAACGTGCGCAAGGAGACGGTG |
| | | CACAACTGGCGCGACTACCTCCGCCTGCACTGCCATCCCCTCGACGAGTTCCTGCCCGATTGGCCGTCCAACCCGCC |
| | | CGATTTCAAGTATGTTGGCTGCTTCCACGATTTGAATTCAATTATTACTTTTTATATTTAAAAACGAGCTGAGGCAAA |
| | | TTGCTAGTGCGAACTGCGAAGTAGTATGTGGGGGGTGGAAGTAGCTCATGACGCCCACCATTCTGGTAAGGGGGAAATT |
| | | AGACATCTGCATTTCAATTTTCAAATGCGTAGTGGTCGAAAGAAAGGAAAACGCTCAGTTTTAGGAAAGGAAACGATCTGAG |
| | | GCTGACGACTGGGTACCCACTCCACTTGTAGTGGCATAAGCACACTACTCTCTCTCTCTCTCTTTCTTTCAAATGTGTACTAA |
| | | TTGAAATATAAGGGAGGAACC |
| 78 | 3900-01- | CTTGGATCCTGCGATCCGCCGCTGGAGCGTAACCACCGCGTCCACATGCTGCTTCATTGGTGCCGGCGCGGAGGCTG |
| | B10 | CTTAGCTCGCCGCCGAGGACTACTTTGCCCGCCACGGTTACGCTGGGTACCCACTCCACTTGTAGTGGCATAAGCACAC |
| | | TACTCTCTCTCTCTCTCTTTCTTTCAAATGTGTACTAATTGAAATATAAGGGAGGAACC |
| 79 | 3900-01- | CTTGGATCCTGCGATCCGCCGCTGGAGCGTAACCACCGCGTCCACATGCTGCTTCATTGGTGCCGGCGCGGAGGCTG |
| | B11 | CTTAGCTCGCCGCCGAGGACTACTTTGCCCGCTACTCTCTCTCTCTCTCTTTCTTTCAAATGTGTACTAATTGAAATATAAG |
| | | GGAGGAACC |
| 80 | 3900-01-C4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTACCCACTCCACTTGTAGTGGCATAAGCACACTACTCTCTCTC |
| | | TCTCTCTTTCTTTCAAATGTGTACTAATTGAAATATAAGGGAGGAACC |
| 81 | 3900-01-D4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCA[GCTGGAGCGTAACCACCGCGTC]CACACTACTCTCTCTCTCTCT |
| | | CTTTCTTTCAAATGTGTACTAATTGAAATATAAGGGAGGAACC |
| 82 | 3900-01-D8 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGACTGGGTACCCACTCCACTTGTAGTGGCATAAGCACACTACTC |
| | | TCTCTCTCTCTCTTTCTTTCAAATGTGTACTAATTGAAATATAAGGGAGGAACC |
| 83 | 3900-01-D9 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGC[TCC]ACGACTGGGTACCCACTCCACTTGTAGTGGCATAAGCACACTA |
| | | CTCTCTCTCTCTCTCTTTCTTTCAAATGTGTACTAATTGAAATATAAGGGAGGAACC |
| 84 | 3900-01-E7 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGGGTACCCACTCCACTTGTAGTGGCATAAGCACACTAC |
| | | TCTCTCTCTCTCTCTTTCTTTCAAATGTGTACTAATTGAAATATAAGGGAGGAACC |
| 85 | 3900-01-H5 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTGGTACCCACTCCACTTGTAGTGGCATAAGCACACTACTCTC |
| | | TCTCTCTCTCTTTCTTTCAAATGTGTACTAATTGAAATATAAGGGAGGAACC |
| 86 | 4449-01-E8 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGTACCCACTCCACTTGTAGTGGCATAAGCACACTACTC |
| | | TCTCTCTCTCTCTTTCTTTCAAATGTGTACTAATTGAAATATAAGGGAGGAACC |
| 87 | 4449-01-G2 | CTTGGATCCTGCGATCCGCCGCTGGAGCGTAACCACCGCGTCCACATGCTGCTTCATTGGTGCCGGCGCGGAGGCTG |
| | | CTTAGCTC[ATGACTGCTTAGCTC]GCTGACGACTGGGTACCCACTCCACTTGTAGTGGCATAAGCACACTACTCTCTCTCT |
| | | CTCTCTTTCTTTCAAATGTGTACTAATTGAAATATAAGGGAGGAACC |
| 88 | 4449-01-B8 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCAAGCACACTACTCTCTCTCTCTCTCTTTCTTTCAAATGTGTACTAATTGAAA |
| | | TATAAGGGAGGAACC |
| 89 | 4449-01-F9 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTGGGTACCCACTCCACTTGTAGTGGCATAAGCACACTACTCT |
| | | CTCTCTCTCTCTTTCTTTCAAATGTGTACTAATTGAAATATAAGGGAGGAACC |
| 90 | 4449-01-A7 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCTCCACTTGTAGTGGCATAAGCACACTACTCTCTCTCTCTCTCTTTCTTT |
| | | CAAATGTGTACTAATTGAAATATAAGGGAGGAACC |
| 91 | 4449-01- | CTTGGATCCTGCGATCCGCCGCTGGAGCGTAACCACCGCGTCCACATGCTGCTTCATTGGTGCCGGCGCGGAGGCTG |
| | B11 | CTTAGCTCGCCGCCGAGGACTACTTACTTGTAGTGGCATAAGCACACTACTCTCTCTCTCTCTCTTTCTTTCAAATGTGTAC |
| | | TAATTGAAATATAAGGGAGGAACC |
| 92 | 4449-01-C6 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTGAGGGGTGACGACTGGGTACCCACTCCACTTGTAGTGGC |
| | | ATAAGCACACTACTCTCTCTCTCTCTCTTTCTTTCAAATGTGTACTAATTGAAATATAAGGGAGGAACC |
| 93 | 4449-01-F5 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGGGTACCCACTCCACTTGTAGTGGCATAAGCACACTAC |
| | | TCTCTCTCTCTCTCTTTCTTTCAAATGTGTACTAATTGAAATATAAGGGAGGAACC |
| 94 | 3900-01- | CTTGGATCCTGCGATCCGCCGCTGGAGCGTAACCACCGCGTCCACATGCTGCTTCATTGGTGCCGGCGCGGAGGCTG |
| | A10 | CTTAGCTCGCCGCCGAGGACTACTTTGCCCGCCACGGTTACGtaaaactgagcgttttcctttctttcgaccactacgcatttgaaaattgaaatgcag |
| | | atgtctaatttcccccttaccagaatggtgggcgtcatgagctacttccaccccccacatactacttcgcagttcgcactagcaatttgcctcagctcgtttttaaatataaaaagtaataattga |
| | | attcaaatcgtggaagcagccaacatacttgaaatcggggggttggacggccaatcgggcaggaactcgtcgaggggatggcagtgcaggcggaggtagtcgcgccagttgtgcaccgtctc |
| | | cttgcgcacgttgaagctggtggacagccggatcttcctggcggggtcgtcggagtagagcttggccttctcctcggggggcagccggaagaagacgcgccccgcggccatcaccgccgc |
| | | gaccagggcggcgtgtatcccgtggttgacgacctggaagaagccgtgcgagcggcaggcgtcgccgacggcggccacgaccgcgccgcggtcggggctgcccaggtccaccacgg |
| | | ggatgcgcgcgccggtcacgacctccgcgaggcgcgggcgctccggctccggccgcacgtagctccccggcagcgtgtcgtgcacggccgtcgacaggaggtgctccgccatgccgcc |
| | | ggcgccgtgcgtgcctacggaggtcggtcggtcctgcgctgcgcttgcgtgcagcagtccggcgcggcagcttggtgtcgtgcgcggttccgcctccagtctagcatggctctcccagggg |
| | | ccgccgtcgcgcgctctttatagcggccgggaagccggacgcgtaacctggtgtctggtggtgactgctccgtggctcggcgtgggcgttccggcaggcagcgggcccgtggcgtcagcc |
| | | gtgacgacgcgacgacgaccgagaagaggttgaccccccgcggtcgttgggtggactgatgactgatatcccctcgcgtcatcgccgtggcggtgagtgacgtcaccCTGGGTACC |
| | | CACTCCACTTGTAGTGGCATAAGCACACTACTCTCTCTCTCTCTCTTTCTTTCAAATGTGTACTAATTGAAATATAAGGGAGGA |
| | | ACC |
| 95 | 3900-01- | CTTGGATCCTGCGATCCGCCGCTGGAGCGTAACCACCGCGTCCACATGCTGCTTCATTGGTGCCGGCGCGGAGGCTG |
| | C10 | CTTAGCTCGCCGCCGAGGtttcctttcctaaaactgagcgttttcctttctttcgaccactacgcatttgaaaattgaaatgcagatgtctaatttcccccttaccagaatggtgg |
| | | gcgtcatgagctacttccaccccccacatactacttcgcagttcgcactagcaatttgcctcagctcgtttttaaatataaaaagtaataattgaattcaaatcgtggaagcagccaacatacttg |
| | | aaatcgggcgggttggacggccaatcgggcaggaactcgtcgaggggatggcagtgcaggcggaggtagtcgcgccagttgtgcaccgtctccttgcgcacgttgaagctggtggacagccg |
| | | gatcttcctggcggggtcgtcggagtagagcttggccttctcctcggggggcagccggaagaagacgcgccccgcggccatcaccgccgcgaccagggggcgtgtatcccgtggttgac |
| | | gacctggaagaagccgtgcgagcggcaggcgtcgccgacggcggccacgaccgcgccgcggtcggggctgcccaggtccaccacggggatgcgcgcgccggtcacgacctccgcg |
| | | aggcgcgggcgctccggctccggccgcacgtagctccccggcagcgtgtcgtgcacggccgtcgacaggaggtgctccgccatgccgccggcgccgtgcgtgcctacggaggtcggtc |
| | | ggtcctgcgctgcgcttgcgtgcagcagtccggcgcggcagcttggtgtcgtgcgcggttccgcctccagtctagcatggctctcccaggggccgccgtcgcgcgctctttatagcggccgg |
| | | gaagccggacgcgtaacctggtgtctggtggtgactgctccgtggctcggcgtgggcgttccggcaggcagcgggcccgtggcgtcagccgtgacgacgcgacgacgaccgagaagag |
| | | gttgaccccccgcggtcgttgggtggactgatgactgatatcccctcgcgtcatcgccgtggcggtgagtgacgtcaccacGACTGGGTACCCACTCCACTTGTAGTGG |
| | | CATAAGCACACTACTCTCTCTCTCTCTCTTTCTTTCAAATGTGTACTAATTGAAATATAAGGGAGGAACC |
| 96 | 3900-01-D6 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGcctttcctaaaactgagcgttttcctttctttcgaccactacgcatttgaaaattgaa |
| | | atgcagatgtctaatttcccccttaccagaatggtgggcgtcatgagctacttccaccccccacatactacttcgcagttcgcactagcaatttgcctcagctcgtttttaaatataaaaagtaa |
| | | taattgaattcaaatcgtggaagcagccaacatacttgaaatcggggggttggacggccaatcgggcaggaactcgtcgaggggatggcagtgcaggggaggtagtcgcgccagttgtgcac |
| | | cgtctccttgcgcacgttgaagctggtggacagccggatcttcctggcggggtcgtcggagtagagcttggccttctcctcggggggcagccggaagaagacgcgccccgcggccatcacc |
| | | gccgcgaccagggcggcgtgtatcccgtggttgacgacctggaagaagccgtgcgagcggcaggcgtcgccgacggcggccacgaccgcgccgcggtcggggctgcccaggtccac |
| | | cacggggatgcgcgcgccggtcacgacctccgcgaggcgcgggcgctccggctccggccgcacgtagctccccggcagcgtgtcgtgcacggccgtcgacaggaggtgctccgccat |
| | | gccgccggcgccgtgcgtgcctacggaggtcggtcggtcctgcgctgcgcttgcgtgcagcagtccggcgcggcagcttggtgtcgtgcgcggttccgcctccagtctagcatggctctccc |
| | | aggggccgccgtcgcgcgctctttatagcggccgggaagccggacgcgtaacctggtgtctggtggtgactgctccgtggctcggcgtgggcgttccggcaggcagcgggcccgtggcgt |
| | | cagccgtgacgacgcgacgacgaccgagaagaggttgaccccccgcggtcgttgggtggactgatgactgatatcccctcgcgtcatcgccgtggcggtgagtgacgtcaccacGACT |
| | | GGGTACCCACTCCACTTGTAGTGGCATAAGCACACTACTCTCTCTCTCTCTCTTTCTTTCAAATGTGTACTAATTGAAATATAA |
| | | GGGAGGAACC |
| 97 | 3900-01-D7 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCAcctttcctaaaactgagcgttttcctttctttcgaccactacgcatttgaaaattgaaatgcagatgtcta |
| | | atttcccccttaccagaatggtgggcgtcatgagctacttccaccccccacatactacttcgcagttcgcactagcaatttgcctcagctcgtttttaaatataaaaagtaataattgaattcaaa |
| | | tcgtggaagcagccaacatacttgaaatcggggggttggacggccaatcgggcaggaactcgtcgaggggatggcagtgcaggcggaggtagtcgcgccagttgtgcaccgtctccttgcgc |
| | | acgttgaagctggtggacagccggatcttcctggcggggtcgtcggagtagagcttggccttctcctcggggggcagccggaagaagacgcgccccgcggccatcaccgccgcgaccag |
| | | ggcggcgtgtatcccgtggttgacgacctggaagaagccgtgcgagcggcaggcgtcgccgacggcggccacgaccgcgccgcggtcggggctgcccaggtccaccacggggatgcg |
| | | cgcgccggtcacgacctccgcgaggcgcgggcgctccggctccggccgcacgtagctccccggcagcgtgtcgtgcacggccgtcgacaggaggtgctccgccatgccgccggcgcc |
| | | gtgcgtgcctacggaggtcggtcggtcctgcgctgcgcttgcgtgcagcagtccggcgcggcagcttggtgtcgtgcgcggttccgcctccagtctagcatggctctcccaggggccgccgt |
| | | cgcgcgctctttatagcggccgggaagccggacgcgtaacctggtgtctggtggtgactgctccgtggctcggcgtgggcgttccggcaggcagcgggcccgtggcgtcagccgtgacga |
| | | cgcgacgacgaccgagaagaggttgaccccccgcggtcgttgggtggactgatgactgatatcccctcgcgtcatcgccgtggcggtgagtgacgtcaccGACTGGGTACCCAC |
| | | TCCACTTGTAGTGGCATAAGCACACTACTCTCTCTCTCTCTCTTTCTTTCAAATGTGTACTAATTGAAATATAAGGGAGGAACC |
| 98 | 3900-01-E3 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGtttcctttcctaaaactgagcgttttccttttttcgaccactacgcatttgaaaattgaaatgcag |
| | | atgtctaatttcccccttaccagaatggtgggcgtcatgagctacttccaccccccacatactacttcgcagttcgcactagcaatttgcctcagctcgtttttaaatataaaaagtaataattga |
| | | attcaaatcgtggaagcagccaacatacttgaaatcggggggttggacggccaatcgggcaggaactcgtcgaggggatggcagtgcaggcggaggtagtcgcgccagttgtgcaccgtctc |
| | | cttgcgcacgttgaagctggtggacagccggatcttcctggcggggtcgtcggagtagagcttggccttctcctcggggggcagccggaagaagacgcgccccgcggccatcaccgccgc |
| | | gaccagggcggcgtgtatcccgtggttgacgacctggaagaagccgtgcgagcggcaggcgtcgccgacggcggccacgaccgcgccgcggtcggggctgcccaggtccaccacgg |
| | | ggatgcgcgcgccggtcacgacctccgcgaggcgcgggcgctccggctccggccgcacgtagctccccggcagcgtgtcgtgcacggccgtcgacaggaggtgctccgccatgccgcc |
| | | ggcgccgtgcgtgcctacggaggtcggtcggtcctgcgctgcgcttgcgtgcagcagtccggcgcggcagcttggtgtcgtgcgcggttccgcctccagtctagcatggctctcccagggg |
| | | ccgccgtcgcgcgctctttatagcggccgggaagccggacgcgtaacctggtgtctggtggtgactgctccgtggctcggcgtggggttccggcaggcagcgggcccgtggcgtcagcc |
| | | gtgacgacgcgacgacgaccgagaagaggttgaccccccgcggtcgttgggtggactgatgactgatatcccctcgcgtcatcgccgtggcggtgagtgacgtcaccTGACGACTG |
| | | GGTACCCACTCCACTTGTAGTGGCATAAGCACACTACTCTCTCTCTCTCTCTTTCTTTCAAATGTGTACTAATTGAAATATAAG |
| | | GGAGGAACC |
| 99 | 3900-01-E4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGtttcctttcctaaaactgagcgttttcctttctttcgaccactacgcatttgaaaattgaaatgcagatgtctaatttccccctt |
| | | accagaatggtgggcgtcatgagctacttccaccccccacatactacttcgcagttcgcactagcaatttgcctcagctcgtttttaaatataaaaagtaataattgaattcaaatcgtggaagca |
| | | gccaacatacttgaaatcggggggttggacggccaatcgggcaggaactcgtcgaggggatggcagtgcaggcggaggtagtcgcgccagttgtgcaccgtctccttgcgcacgttgaagc |
| | | tggtggacagccggatcttcctggcggggtcgtcggagtagagcttggccttctcctcggggggcagccggaagaagacgcgccccgcggccatcaccgccgcgaccagggggcgtgt |
| | | atcccgtggttgacgacctggaagaagccgtgcgagcggcaggcgtcgccgacggcggccacgaccgcgccgcggggggctgcccaggtccaccacggggatgcgcgcgccggt |
| | | cacgacctccgcgaggcgcgggcgctccggctccggccgcacgtagctccccggcagcgtgtcgtgcacggccgtcgacaggaggtgctccgccatgccgccggcgccgtgcgtgcct |
| | | acggaggtcggtcggtcctgcgctgcgcttgcgtgcagcagtccggcgcggcagcttggtgtcgtgcgcggttccgcctccagtctagcatggctctcccaggggccgccgtcgcgcgctct |
| | | ttatagcggccgggaagccggacgcgtaacctggtgtctggtggtgactgctccgtggctcggcgtgggcgttccggcaggcagcgggcccgtggcgtcagccgtgacgacgcgacgac |
| | | gaccgagaagaggttgaccccccgcggtcgttgggtggactgatgactgatatcccctcgcgtcatcgccgtggcggtgagtgacgtcaccaACCCACTCCACTTGTAGTGG |
| | | CATAAGCACACTACTCTCTCTCTCTCTCTTTCTTTCAAATGTGTACTAATTGAAATATAAGGGAGGAACC |
| 100 | 3900-01-E5 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGtttcctttcctaaaactgagcgttttcctttctttcgaccactacgcatttgaaaattgaaatgcag |
| | | atgtctaatttcccccttaccagaatggtgggcgtcatgagctacttccaccccccacatactacttcgcagttcgcactagcaatttgcctcagctcgtttttaaatataaaaagtaataattga |
| | | attcaaatcgtggaagcagccaacatacttgaaatcggggggttggacggccaatcgggcaggaactcgtcgaggggatggcagtgcaggcggaggtagtcgcgccagttgtgcaccgtctc |
| | | cttgcgcacgttgaagctggtggacagccggatcttcctggggggtcgtcggagtagagcttggccttctcctcggggggcagccggaagaagacgcgccccgcggccatcaccgccgc |
| | | gaccagggggcgtgtatcccgtggttgacgacctggaagaagccgtgcgagcggcaggcgtcgccgacggcggccacgaccgcgccgcggtcggggctgcccaggtccaccacgg |
| | | ggatgcgcgcgccggtcacgacctccgcgaggcgcgggcgctccggctccggccgcacgtagctccccggcagcgtgtcgtgcacggccgtcgacaggaggtgctccgccatgccgcc |
| | | ggcgccgtgcgtgcctacggaggtcggtcggtcctgcgctgcgcttgcgtgcagcagtccggcgcggcagcttggtgtcgtgcgggttccgcctccagtctagcatggctctcccagggg |
| | | ccgccgtcgcgcgctctttatagcggccgggaagccggacgcgtaacctggtgtctggtggtgactgctccgtggctcggcgtgggcgttccggcaggcagcgggcccgtggcgtcagcc |
| | | gtgacgacgcgacgacgaccgagaagaggttgaccccccgcggtcgttgggtggactgatgactgatatcccctcgcgtcatcgccgtggcggtgagtgacgtcacACGACTGGGT |
| | | ACCCACTCCACTTGTAGTGGCATAAGCACACTACTCTCTCTCTCTCTCTTTCTTTCAAATGTGTACTAATTGAAATATAAGGGA |
| | | GGAACC |
| 101 | 3900-01-E7 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACatcgtttcctttcctaaaactgagcgttttcctttctttcgaccactacgcatttgaaaatt |
| | | gaaatgcagatgtctaatttcccccttaccagaatggtgggcgtcatgagctacttccaccccccacatactacttcgcagttcgcactagcaatttgcctcagctcgtttttaaatataaaaag |
| | | taataattgaattcaaatcgtggaagcagccaacatacttgaaatcggggggttggacggccaatcgggcaggaactcgtcgaggggatggcagtgcaggcggaggtagtcgcgccagttgt |
| | | gcaccgtctccttgcgcacgttgaagctggtggacagccggatcttcctggcggggtcgtcggagtagagcttggccttctcctcggggggcagccggaagaagacgcgccccgcggccat |
| | | caccgccgcgaccagggcggcgtgtatcccgtggttgacgacctggaagaagccgtgcgagcggcaggcgtcgccgacggcggccacgaccgcgccgcggtcggggctgcccaggt |
| | | ccaccacggggatgcgcgcgccggtcacgacctccgcgaggcgcgggcgctccggctccggccgcacgtagctccccggcagcgtgtcgtgcacggccgtcgacaggaggtgctccg |
| | | ccatgccgccggcgccgtgcgtgcctacggaggtcggtcggtcctgcgctgcgcttgcgtgcagcagtccggcgcggcagcttggtgtcgtgcgcggttccgcctccagtctagcatggctc |
| | | tcccaggggccgccgtcgcgcgctctttatagcggccgggaagccggacgcgtaacctggtgtctggtggtgactgctccgtggctcggcgtgggcgttccggcaggcagcgggcccgtg |
| | | gcgtcagccgtgacgacgcgacgacgaccgagaagaggttgaccccccgcggtcgttgggtggactgatgactgatatcccctcgcgtcatcgccgtggcggtgagtgacgtcaccGCT |
| | | GACGACTGGGTACCCACTCCACTTGTAGTGGCATAAGCACACTACTCTCTCTCTCTCTCTTTCTTTCAAATGTGTACTAATTG |
| | | AAATATAAGGGAGGAACC |
| 102 | 3900-01-F6 | CTTGGATCCTGCGATCCGCCGCTGGAGCGTAACCACCGCGTCCACATGCTGCTTCATTGGTGCCGGCGCGGAGGCTG |
| | | CTT[G]GCgtttcctttcctaaaactgagcgttttcctttctttcgaccactacgcatttgaaaattgaaatgcagatgtctaatttcccccttaccagaatggtgggcgtcatgagctacttc |
| | | caccccccacatactacttcgcagttcgcactagcaatttgcctcagctcgtttttaaatataaaaagtaataattgaattcaaatcgtggaagcagccaacatacttgaaatcggggggttggac |
| | | ggccaatcgggcaggaactcgtcgaggggatggcagtgcaggcggaggtagtcgcgccagttgtgcaccgtctccttgcgcacgttgaagctggtggacagccggatcttcctggcgggg |
| | | tcgtcggagtagagcttggccttctcctcggggggcagccggaagaagacgcgccccgcggccatcaccgccgcgaccagggcggcgtgtatcccgtggttgacgacctggaagaagcc |
| | | gtgcgagcggcaggcgtcgccgacggcggccacgaccgcgccgcggtcggggctgcccaggtccaccacggggatgcgcgcgccggtcacgacctccgcgaggcgcgggcgctcc |
| | | ggctccggccgcacgtagctccccggcagcgtgtcgtgcacggccgtcgacaggaggtgctccgccatgccgccggcgccgtgcgtgcctacggaggtcggtcggtcctgcgctgcgctt |
| | | gcgtgcagcagtccggcgcggcagcttggtgtcgtgcgcggttccgcctccagtctagcatggctctcccaggggccgccgtcgcgcgctctttatagcggccgggaagccggacgcgtaa |
| | | cctggtgtctggtggtgactgctccgtggctcggcgtgggcgttccggcaggcagcgggcccgtggcgtcagccgtgacgacgcgacgacgaccgagaagaggttgaccccccgcggtc |
| | | gttgggtggactgatgactgatatcccctcgcgtcatcgccgtggcggtgagtgacgtcaccacccACGACTGGGTACCCACTCCACTTGTAGTGGCATAAGCA |
| | | CACTACTCTCTCTCTCTCTCTTTCTTTCAAATGTGTACTAATTGAAATATAAGGGAGGAACC |
| 103 | 3900-01-G6 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACtttcctttcctaaaactgagcgttttcctttctttcgaccactacgcatttgaaaattgaaatgcagatgtctaatttcccccttaccaga |
| | | atggtgggcgtcatgagctacttccaccccccacatactacttcgcagttcgcactagcaatttgcctcagctcgtttttaaatataaaaagtaataattgaattcaaatcgtggaagcagccaac |
| | | atacttgaaatcggggggttggacggccaatcgggcaggaactcgtcgaggggatggcagtgcaggcggaggtagtcgcgccagttgtgcaccgtctccttgcgcacgttgaagctggtgg |
| | | acagccggatcttcctggggggtcgtcggagtagagcttggccttctcctcggggggcagccggaagaagacgcgccccgcggccatcaccgccgcgaccagggggcgtgtatcccg |
| | | tggttgacgacctggaagaagccgtgcgagcggcaggcgtcgccgacggcggccacgaccgcgccgcggtcggggctgcccaggtccaccacggggatgcgcgcgccggtcacgac |
| | | ctccgcgaggcgcgggcgctccggctccggccgcacgtagctccccggcagcgtgtcgtgcacggccgtcgacaggaggtgctccgccatgccgccggcgccgtgcgtgcctacggag |
| | | gtcggtcggtcctgcgctgcgcttgcgtgcagcagtccggcgcggcagcttggtgtcgtgcgcggttccgcctccagtctagcatggctctcccaggggccgccgtcgcgcgctctttatagc |
| | | ggccgggaagccggacgcgtaacctggtgtctggtggtgactgctccgtggctcggcgtgggcgttccggcaggcagcgggcccgtggcgtcagccgtgacgacgcgacgacgaccga |
| | | gaagaggttgaccccccgcggtcgttgggtggactgatgactgatatcccctcgcgtcatcgccgtggcggtgagtgacgtcGTACCCACTCCACTTGTAGTGGCATAA |
| | | GCACACTACTCTCTCTCTCTCTCTTTCTTTCAAATGTGTACTAATTGAAATATAAGGGAGGAACC |
| 104 | 3900-01- | CTTGGATCCTGCGATCCGCCGCTGGAGCGTAACCACCGCGTCCACATGCTGCTTCATTGGTGCCGGCGCGGAGGCTG |
| | H3-1 | CTTAGCTCGCCGCCGAGGACTACTTTGCCCGCCACGGTTctttcctaaaactgagcgttttcctttctttcgaccactacgcatttgaaaattgaaatgcag |
| | | atgtctaatttcccccttaccagaatggtgggcgtcatgagctacttccaccccccacatactacttcgcagttcgcactagcaatttgcctcagctcgtttttaaatataaaaagtaataattga |
| | | attcaaatcgtggaagcagccaacatacttgaaatcggggggttggacggccaatcgggcaggaactcgtcgaggggatggcagtgcaggcggaggtagtcgcgccagttgtgcaccgtctc |
| | | cttgcgcacgttgaagctggtggacagccggatcttcctggcggggtcgtcggagtagagcttggccttctcctcggggggcagccggaagaagacgcgccccgcggccatcaccgccgc |
| | | gaccagggcggcgtgtatcccgtggttgacgacctggaagaagccgtgcgagcggcaggcgtcgccgacggcggccacgaccgcgccgcggtcggggctgcccaggtccaccacgg |
| | | ggatgcgcgcgccggtcacgacctccgcgaggcgcgggcgctccggctccggccgcacgtagctccccggcagcgtgtcgtgcacggccgtcgacaggaggtgctccgccatgccgcc |
| | | ggcgccgtgcgtgcctacggaggtcggtcggtcctgcgctgcgcttgcgtgcagcagtccggcgcggcagcttggtgtcgtgcgcggttccgcctccagtctagcatggctctcccagggg |
| | | ccgccgtcgcgcgctctttatagcggccgggaagccggacgcgtaacctggtgtctggtggtgactgctccgtggctcggcgtgggcgttccggcaggcagcgggcccgtggcgtcagcc |
| | | gtgacgacgcgacgacgaccgagaagaggttgaccccccgcggtcgttgggtggactgatgactgatatcccctcgcgtcatcgccgtggcggtgagtgacgtcaccTGGGTACCC |
| | | ACTCCACTTGTAGTGGCATAAGCACACTACTCTCTCTCTCTCTCTTTCTTTCAAATGTGTACTAATTGAAATATAAGGGAGGAA |
| | | CC |
| 105 | 3900-01- | CTTGGATCCTGCGATCCGCCGCTGGAGCGTAACCACCGCGTCCACATGCTGCTTCATTGGTGCCGGCGCGGAGGCTG |
| | H3-4 | CTTAGCTCGCCGCCGAGGACT[G]Ccgtttcctttcctaaaactgagcgttttcctttctttcgaccactacgcatttgaaaattgaaatgcagatgtctaatttcccccttac |
| | | cagaatggtgggcgtcatgagctacttccaccccccacatactacttcgcagttcgcactagcaatttgcctcagctcgtttttaaatataaaaagtaataattgaattcaaatcgtggaagcag |
| | | ccaacatacttgaaatcggggggttggacggccaatcgggcaggaactcgtcgaggggatggcagtgcaggcggaggtagtcgcgccagttgtgcaccgtctccttgcgcacgttgaagctg |
| | | gtggacagccggatcttcctggcggggtcgtcggagtagagcttggccttctcctcggggggcagccggaagaagacgcgccccgcggccatcaccgccgcgaccagggcggcgtgtat |
| | | cccgtggttgacgacctggaagaagccgtgcgagcggcaggcgtcgccgacggcggccacgaccgcgccgcggtcggggctgcccaggtccaccacggggatgcgcgcgccggtca |
| | | cgacctccgcgaggcgcgggcgctccggctccggccgcacgtagctccccggcagcgtgtcgtgcacggccgtcgacaggaggtgctccgccatgccgccggcgccgtgcgtgcctac |
| | | ggaggtcggtcggtcctgcgctgcgcttgcgtgcagcagtccggcgcggcagcttggtgtcgtgcgcggttccgcctccagtctagcatggctctcccaggggccgccgtcgcgcgctcttt |
| | | atagcggccgggaagccggacgcgtaacctggtgtctggtggtgactgctccgtggctcggcgtgggcgttccggcaggcagcgggcccgtggcgtcagccgtgacgacgcgacgacg |
| | | accgagaagaggttgaccccccgcggtcgttgggtggactgatgactgatatcccctcgcgtcatcgccgtggcggtgagtgacgtcaccaccGGGTACCCACTCCACTTGT |
| | | AGTGGCATAAGCACACTACTCTCTCTCTCTCTCTTTCTTTCAAATGTGTACTAATTGAAATATAAGGGAGGAACC |
| 106 | 3900-01-H5 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTtcctaaaactgagcgttttcctttctttcgaccactacgcatttgaaaattgaaatgcagat |
| | | gtctaatttcccccttaccagaatggtgggcgtcatgagctacttccaccccccacatactacttcgcagttcgcactagcaatttgcctcagctcgtttttaaatataaaaagtaataattgaat |
| | | tcaaatcgtggaagcagccaacatacttgaaatcggggggttggacggccaatcgggcaggaactcgtcgaggggatggcagtgcaggcggaggtagtcgcgccagttgtgcaccgtctcctt |
| | | gcgcacgttgaagctggtggacagccggatcttcctggggggtcgtcggagtagagcttggccttctcctcggggggcagccggaagaagacgcgccccgcggccatcaccgccgcga |
| | | ccagggcggcgtgtatcccgtggttgacgacctggaagaagccgtgcgagcggcaggcgtcgccgacggcggccacgaccgcgccgcggtcggggctgcccaggtccaccacgggg |
| | | atgcgcgcgccggtcacgacctccgcgaggcgcgggcgctccggctccggccgcacgtagctccccggcagcgtgtcgtgcacggccgtcgacaggaggtgctccgccatgccgccgg |
| | | cgccgtgcgtgcctacggaggtcggtcggtcctgcgctgcgcttgcgtgcagcagtccggcgcggcagcttggtgtcgtgcgcggttccgcctccagtctagcatggctctcccaggggcc |
| | | gccgtcgcgcgctctttatagcggccgggaagccggacgcgtaacctggtgtctggtggtgactgctccgtggctcggcgtgggcgttccggcaggcagcgggcccgtggcgtcagccgt |
| | | gacgacgcgacgacgaccgagaagaggttgaccccccgcggtcgttgggtggactgatgactgatatcccctcgcgtcatcgccgtggcggtgagtgacgtcacc[g]ccACTTGTA |
| | | GTGGCATAAGCACACTACTCTCTCTCTCTCTCTTTCTTTCAAATGTGTACTAATTGAAATATAAGGGAGGAACC |
| 107 | 4449-01-A3 | CTTGGATCCTGCGATCCGCCGCTGGAGCGTAACCACCGCGTCCACATGCTGCTTCATTGGTGC[G]cctaaaactgagcgttttcc |
| | | tttctttcgaccactacgcatttgaaaattgaaatgcagatgtctaatttcccccttaccagaatggtgggcgtcatgagctacttccaccccccacatactacttcgcagttcgcactagcaat |
| | | ttgcctcagctcgtttttaaatataaaaagtaataattgaattcaaatcgtggaagcagccaacatacttgaaatcggggggttggacggccaatcgggcaggaactcgtcgaggggatggcagt |
| | | gcaggcggaggtagtcgcgccagttgtgcaccgtctccttgcgcacgttgaagctggtggacagccggatcttcctggggggtcgtcggagtagagcttggccttctcctcggggggcagc |
| | | cggaagaagacgcgccccgcggccatcaccgccgcgaccagggggcgtgtatcccgtggttgacgacctggaagaagccgtgcgagcggcaggcgtcgccgacggcggccacgac |
| | | cgcgccgcggtcggggctgcccaggtccaccacggggatgcgcgcgccggtcacgacctccgcgaggcgcgggcgctccggctccggccgcacgtagctccccggcagcgtgtcgtg |
| | | cacggccgtcgacaggaggtgctccgccatgccgccggcgccgtgcgtgcctacggaggtcggtcggtcctgcgctgcgcttgcgtgcagcagtccggcgcggcagcttggtgtcgtgcg |
| | | cggttccgcctccagtctagcatggctctcccaggggccgccgtcgcgcgctctttatagcggccgggaagccggacgcgtaacctggtgtctggtggtgactgctccgtggctcggcgtgg |
| | | gcgttccggcaggcagcgggcccgtggcgtcagccgtgacgacgcgacgacgaccgagaagaggttgaccccccgcggtcgttgggtggactgatgactgatatcccctcgcgtcatcgc |
| | | cgtggcggtgagtgacgtcaccAGGCTGACGACTGGGTACCCACTCCACTTGTAGTGGCATAAGCACACTACTCTCTCTCTCTCTCTTT |
| | | CTTTCAAATGTGTACTAATTGAAATATAAGGGAGGAACC |
| 108 | 4449-01- | CTTGGATCCTGCGATCCGCCGCTGGAGCGTAACCACCGCGTCCACATGCTGCTTCATTGGTGCCGGCGCGGAGGCTG |
| | A9-1 | CTTAGCTCGCCGCCGAGGACTACTTTGCCCGCCatcgtttcctttcctaaaactgagcgttttcctttctttcgaccactacgcatttgaaaattgaaatgcagat |
| | | gtctaatttcccccttaccagaatggtgggcgtcatgagctacttccaccccccacatactacttcgcagttcgcactagcaatttgcctcagctcgtttttaaatataaaaagtaataattgaat |
| | | tcaaatcgtggaagcagccaacatacttgaaatcggggggttggacggccaatcgggcaggaactcgtcgaggggatggcagtgcaggcggaggtagtcgcgccagttgtgcaccgtctcctt |
| | | gcgcacgttgaagctggtggacagccggatcttcctggcggggtcgtcggagtagagcttggccttctcctcggggggcagccggaagaagacgcgccccgcggccatcaccgccgcga |
| | | ccagggcggcgtgtatcccgtggttgacgacctggaagaagccgtgcgagcggcaggcgtcgccgacggcggccacgaccgcgccgcggtcggggctgcccaggtccaccacgggg |
| | | atgcgcgcgccggtcacgacctccgcgaggcgcgggcgctccggctccggccgcacgtagctccccggcagcgtgtcgtgcacggccgtcgacaggaggtgctccgccatgccgccgg |
| | | cgccgtgcgtgcctacggaggtcggtcggtcctgcgctgcgcttgcgtgcagcagtccggcgcggcagcttggtgtgtgcgcggttccgcctccagtctagcatggctctcccaggggcc |
| | | gccgtcgcgcgctctttatagcggccgggaagccggacgcgtaacctggtgtctggtggtgactgctccgtggctcggcgtgggcgttccggcaggcagcgggcccgtggcgtcagccgt |
| | | gacgacgcgacgacgaccgagaagagcttgaccccccgcggtcgttgggtggactgatgactgatatcccctcgcgtcatcgccgtggcggtgagtgacgtcaACTGGGTACCCA |
| | | CTCCACTTGTAGTGGCATAAGCACACTACTCTCTCTCTCTCTCTTTCTTTCAAATGTGTACTAATTGAAATATAAGGGAGGAAC |
| | | C |
| 109 | 4449-01- | CTTGGATCCTGCGATCCGCCGCTGGAGCGTAACCACCGCGTCCACATGCTGCTTCATTGGTGCCGGCGCGGAGGCTG |
| | A9-2 | CTTAGCTCGCCGCCGAGGACTACTTTGCCCGCCA[GAATGGTGGGCGTCATGAATGGTGCGCGGATTTCAAGTATGT |
| | | TGGCT]gttttcctttctttcgaccactacgcatttgaaaattgaaatgcagatgtctaatttcccccttaccagaatgggggcgtcatgagctacttccaccccccacatactacttcgcag |
| | | ttcgcactagcaatttgcctcagctcgtttttaaatataaaaagtaataattgaattcaaatcgtggaagcagccaacatacttgaaatcggggggttggacggccaatcgggcaggaactcgtc |
| | | gaggggatggcagtgcaggcggaggtagtcgcgccagttgtgcaccgtctccttgcgcacgttgaagctggtggacagccggatcttcctggggggtcgtcggagtagagcttggccttct |
| | | cctcggggggcagccggaagaagacgcgccccgcggccatcaccgccgcgaccagggcggcgtgtatcccgtggttgacgacctggaagaagccgtgcgagcggcaggcgtcgccg |
| | | acggcggccacgaccgcgccgcggtcggggctgcccaggtccaccacggggatgcgcgcgccggtcacgacctccgcgaggcgcgggcgctccggctccggccgcacgtagctccc |
| | | cggcagcgtgtcgtgcacggccgtcgacaggaggtgctccgccatgccgccggcgccgtgcgtgcctacggaggtcggtcggtcctgcgctgcgcttgcgtgcagcagtccggcgcggc |
| | | agcttggtgtcgtgcgcggttccgcctccagtctagcatggctctcccaggggccgccgtcgcgcgctctttatagcggccgggaagccggacgcgtaacctggtgtctggtggtgactgctc |
| | | cgtggctcggcgtgggcgttccggcaggcagcgggcccgtggcgtcagccgtgacgacgcgacgacgaccgagaagaggttgaccccccgcggtcgttgggtggactgatgactgatat |
| | | cccctcgcgtcatcgccgtggcggtgagtgacgtcaccAGGCTGACGACTGGGTACCCACTCCACTTGTAGTGGCATAAGCACACTACTCTCTC |
| | | TCTCTCTCTTTCTTTCAAATGTGTACTAATTGAAATATAAGGGAGGAACC |
| 110 | 4449-01-B4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTttcctaaaactgagcgttttcctttctttcgaccactacgcatttgaaaattgaaatgcagat |
| | | gtctaatttcccccttaccagaatggtgggcgtcatgagctacttccaccccccacatactacttcgcagttgcactagcaatttgcctcagctcgtttttaaatataaaaagtaataattgaatt |
| | | caaatcgtggaagcagccaacatacttgaaatcggggggttggacggccaatcgggcaggaactcgtcgaggggatggcagtgcaggcggaggtagtcgcgccagttgtgcaccgtctcctt |
| | | gcgcacgttgaagctggtggacagccggatcttcctggcggggtcgtcggagtagagcttggccttctcctcggggggcagccggaagaagacgcgccccgcggccatcaccgccgcga |
| | | ccagggcggcgtgtatcccgtggttgacgacctggaagaagccgtgcgagcggcaggcgtcgccgacggcggccacgaccgcgccgcggtcggggctgcccaggtccaccacgggg |
| | | atgcgcgcgccggtcacgacctccgcgaggcgcgggcgctccggctccggccgcacgtagctccccggcagcgtgtcgtgcacggccgtcgacaggaggtgctccgccatgccgccgg |
| | | cgccgtgcgtgcctacggaggtcggtcggtcctgcgctgcgcttgcgtgcagcagtccggcgcggcagcttggtgtcgtgcgcggttccgcctccagtctagcatggctctcccaggggcc |
| | | gccgtcgcgcgctctttatagcggccgggaagccggacgcgtaacctggtgtctggtggtgactgctccgtggctcggcgtgggcgttccggcaggcagcgggcccgtggcgtcagccgt |
| | | gacgacgcgacgacgaccgagaagaggttgaccccccgcggtcgttgggtggactgatgactgatatcccctcgcgtcatcgccgtggcgctgagtgacgtcGGGTACCCACTC |
| | | CACTTGTAGTGGCATAAGCACACTACTCTCTCTCTCTCTCTTTCTTTCAAATGTGTACTAATTGAAATATAAGGGAGGAACC |
| 111 | 4449-01-B9 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCAcgtttcctttcctaaaactgagcgttttcctttctttcgaccactacgcatttgaaaattgaaatgcagatg |
| | | tctaatttcccccttaccagaatggtgggcgtcatgagctacttccaccccccacatactacttcgcagttcgcactagcaatttgcctcagctcgtttttaaatataaaaagtaataattgaatt |
| | | caaatcgtggaagcagccaacatacttgaaatcggggggttggacggccaatcgggcaggaactcgtcgaggggatggcagtgcaggcggaggtagtcgcgccagttgtgcaccgtctcctt |
| | | gcgcacgttgaagctggtggacagccggatcttcctggggggtcgtcggagtagagcttggccttctcctcggggggcagccggaagaagacgcgccccgcggccatcaccgccgcga |
| | | ccagggcggcgtgtatcccgtggttgacgacctggaagaagccgtgcgagcggcaggcgtcgccgacggcggccacgaccgcgccgcggtcggggctgcccaggtccaccacgggg |
| | | atgcgcgcgccggtcacgacctccgcgaggcgcgggcgctccggctccggccgcacgtagctccccggcagcgtgtcgtgcacggccgtcgacaggaggtgctccgccatgccgccgg |
| | | cgccgtgcgtgcctacggaggtcggtcggtcctgcgctgcgcttgcgtgcagcagtccggcgcggcagcttggtgtcgtgcgcggttccgcctccagtctagcatggctctcccaggggcc |
| | | gccgtcgcgcgctctttatagcggccgggaagccggacgcgtaacctggtgtctggtggtgactgctccgtggctcggcgtggggttccggcaggcagcgggcccgtggcgtcagccgt |
| | | gacgacgcgacgacgaccgagaagaggttgaccccccgcggtcgttgggtggactgatgactgatatcccctcgcgtcatcgccgtggcggtgagtgacgtcacGACGACTGGGT |
| | | ACCCACTCCACTTGTAGTGGCATAAGCACACTACTCTCTCTCTCTCTCTTTCTTTCAAATGTGTACTAATTGAAATATAAGGGA |
| | | GGAACC |
| 112 | 4449-01-D5 | CTTGGATCCTGCGATCCGCCGCTGGAGCGTAACCACCGCGTCCACATGCTGCTTCATTGGTGC[ATGGCGGAG]ctaaaa |
| | | ctgagcgttttcctttctttcgaccactacgcatttgaaaattgaaatgcagatgtctaatttcccccttaccagaatggtgggcgtcatgagctacttccaccccccacatactacttcgcagt |
| | | tcgcactagcaatttgcctcagctcgtttttaaatataaaaagtaataattgaattcaaatcgtggaagcagccaacatacttgaaatcggggggttggacggccaatcgggcaggaactcgtcga |
| | | ggggatggcagtgcaggcggaggtagtcgcgccagttgtgcaccgtctccttgcgcacgttgaagctggtggacagccggatcttcctggggggtcgtcggagtagagcttggccttctcc |
| | | tcggggggcagccggaagaagacgcgccccgcggccatcaccgccgcgaccagggggcgtgtatcccgtggttgacgacctggaagaagccgtgcgagcggcaggcgtcgccgac |
| | | ggcggccacgaccgcgccgcggtcggggctgcccaggtccaccacggggatgcgcgcgccggtcacgacctccgcgaggcgcgggcgctccggctccggccgcacgtagctccccg |
| | | gcagcgtgtcgtgcacggccgtcgacaggaggtgctccgccatgccgccggcgccgtgcgtgcctacggaggtcggtcggtcctgcgctgcgcttgcgtgcagcagtccggcgcggcag |
| | | cttggtgtcgtgcgcggttccgcctccagtctagcatggctctcccaggggccgccgtcgcgcgctctttatagcggccgggaagccggacgcgtaacctggtgtctggtggtgactgctccg |
| | | tggctcggcgtgggcgttccggcaggcagcgggcccgtggcgtcagccgtgacgacgcgacgacgaccgagaagaggttgaccccccgcggtcgttgggtggactgatgactgatatcc |
| | | cctcgcgtcatcgccgtggcggtgagtgacgtcacAGGCTGACGACTGGGTACCCACTCCACTTGTAGTGGCATAAGCACACTACTCTCTCTC |
| | | TCTCTCTTTCTTTCAAATGTGTACTAATTGAAATATAAGGGAGGAACC |
| 113 | 4449-01-D8 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACctttcctaaaactgagcgttttcctttctttcgaccactacgcatttgaaaattgaaatg |
| | | cagatgtctaatttcccccttaccagaatggtgggcgtcatgagctacttccaccccccacatactacttcgcagttcgcactagcaatttgcctcagctcgtttttaaatataaaaagtaataat |
| | | tgaattcaaatcgtggaagcagccaacatacttgaaatcggggggttggacggccaatcgggcaggaactcgtcgaggggatggcagtgcaggcggaggtagtcgcgccagttgtgcaccg |
| | | tctccttgcgcacgttgaagctggtggacagccggatcttcctggggggtcgtcggagtagagcttggccttctcctcggggggcagccggaagaagacgcgccccgcggccatcaccgc |
| | | cgcgaccagggcggcgtgtatcccgtggttgacgacctggaagaagccgtgcgagcggcaggcgtcgccgacggcggccacgaccgcgccgcggtcggggctgcccaggtccaccac |
| | | ggggatgcgcgcgccggtcacgacctccgcgaggcgcgggcgctccggctccggccgcacgtagctccccggcagcgtgtcgtgcacggccgtcgacaggaggtgctccgccatgcc |
| | | gccggcgccgtgcgtgcctacggaggtcggtcggtcctgcgctgcgcttgcgtgcagcagtccggcgcggcagcttggtgtcgtgcgcggttccgcctccagtctagcatggctctcccag |
| | | gggccgccgtcgcgcgctctttatagcggccgggaagccggacgcgtaacctggtgtctggtggtgactgctccgtggctcggcgtgggcgttccggcaggcagcgggcccgtggcgtca |
| | | gccgtgacgacgcgacgacgaccgagaagaggttgaccccccgcggtcgttgggtggactgatgactgatatcccctcgcgtcatcgccgtggcggtgagtgacgtcaccacTGGGT |
| | | ACCCACTCCACTTGTAGTGGCATAAGCACACTACTCTCTCTCTCTCTCTTTCTTTCAAATGTGTACTAATTGAAATATAAGGGA |
| | | GGAACC |
| 114 | 4449-01-E2 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGGAGGGGTGactgagcgttttcctttctttcgaccactacgcatttgaaaattg |
| | | aaatgcagatgtctaatttcccccttaccagaatggtgggcgtcatgagctacttccaccccccacatactacttcgcagttcgcactagcaatttgcctcagctcgtttttaaatataaaaagt |
| | | aataattgaattcaaatcgtggaagcagccaacatacttgaaatcggggggttggacggccaatcgggcaggaactcgtcgaggggatggcagtgcaggcggaggtagtcgcgccagttgtg |
| | | caccgtctccttgcgcacgttgaagctggtggacagccggatcttcctggggggtcgtcggagtagagcttggccttctcctcggggggcagccggaagaagacgcgccccgcggccatc |
| | | accgccgcgaccagggcggcgtgtatcccgtggttgacgacctggaagaagccgtgcgagcggcaggcgtcgccgacggcggccacgaccgcgccgcggtcggggctgcccaggtc |
| | | caccacggggatgcgcgcgccggtcacgacctccgcgaggcgcgggcgctccggctccggccgcacgtagctccccggcagcgtgtcgtgcacggccgtcgacaggaggtgctccgc |
| | | catgccgccggcgccgtgcgtgcctacggaggtcggtcggtcctgcgctgcgcttgcgtgcagcagtccggcgcggcagcttggtgtcgtgcgcggttccgcctccagtctagcatggctct |
| | | cccaggggccgccgtcgcgcgctctttatagcggccgggaagccggacgcgtaacctggtgtctggtggtgactgctccgtggctcggcgtgggcgttccggcaggcagcgggcccgtg |
| | | gcgtcagccgtgacgacgcgacgacgaccgagaagaggttgaccccccgcggtcgttgggtggactgatgactgatatcccctcgcgtcatcgccgtggcggtgagtgacgtcacTGG |
| | | GTACCCACTCCACTTGTAGTGGCATAAGCACACTACTCTCTCTCTCTCTCTTTCTTTCAAATGTGTACTAATTGAAATATAAGG |
| | | GAGGAACC |
| 115 | 4449-01-E4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTtttcctttcctaaaactgagcgttttcctttctttcgaccactacgcatttgaaaattgaaatgcagatgtctaatttcccccttacc |
| | | agaatgggggcgtcatgagctacttccaccccccacatactacttcgcagttcgcactagcaatttgcctcagctcgtttttaaatataaaaagtaataattgaattcaaatcgtggaagcagcca |
| | | acatacttgaaatcggggggttggacggccaatcgggcaggaactcgtcgaggggatggcagtgcaggcggaggtagtcgcgccagttgtgcaccgtctccttgcgcacgttgaagctgg |
| | | tggacagccggatcttcctggcggggtcgtcggagtagagcttggccttctcctcggggggcagccggaagaagacgcgccccgggccatcaccgccgcgaccagggggcgtgtatc |
| | | ccgtggttgacgacctggaagaagccgtgcgagcggcaggcgtcgccgacggcggccacgaccgcgccgcggtcggggctgcccaggtccaccacggggatgcgcgcgccggtcac |
| | | gacctccgcgaggcgcgggcgctccggctccggccgcacgtagctccccggcagcgtgtcgtgcacggccgtcgacaggaggtgctccgccatgccgccggcgccgtgcgtgcctacg |
| | | gaggtcggtcggtcctgcgctgcgcttgcgtgcagcagtccggcgcggcagcttggtgtcgtgcgcggttccgcctccagtctagcatggctctcccaggggccgccgtcgcgcgctctttat |
| | | agcggccgggaagccggacgcgtaacctggtgtctggtggtgactgctccgtggctcggcgtgggcgttccggcaggcagcgggcccgtggcgtcagccgtgacgacgcgacgacgac |
| | | cgagaagaggttgaccccccgcggtcgttgggtggactgatgactgatatcccctcgcgtcatcgccgtggcggtgagtgacgtcaccacccc[GC]GACGACTGGGTACCCA |
| | | CTCCACTTGTAGTGGCATAAGCACACTACTCTCTCTCTCTCTCTTTCTTTCAAATGTGTACTAATTGAAATATAAGGGAGGAAC |
| | | C |
| 116 | 4449-01-E7 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGG[G]cctttcctaaaactgagcgttttcctttctttcgaccactacgcatttgaaaattgaaatgca |
| | | gatgtctaatttcccccttaccagaatggtgggcgtcatgagctacttccaccccccacatactacttcgcagttcgcactagcaatttgcctcagctcgtttttaaatataaaaagtaataattg |
| | | aattcaaatcgtggaagcagccaacatacttgaaatcggggggttggacggccaatcgggcaggaactcgtcgaggggatggcagtgcaggcggaggtagtcgcgccagttgtgcaccgtct |
| | | ccttgcgcacgttgaagctggtggacagccggatcttcctggggggtcgtcggagtagagcttggccttctcctcggggggcagccggaagaagacgcgccccgcggccatcaccgccg |
| | | cgaccagggcggcgtgtatcccgtggttgacgacctggaagaagccgtgcgagcggcaggcgtcgccgacggcggccacgaccgcgccgcggtcggggctgcccaggtccaccacg |
| | | gggatgcgcgcgccggtcacgacctccgcgaggcgcgggcgctccggctccggccgcacgtagctccccggcagcgtgtcgtgcacggccgtcgacaggaggtgctccgccatgccg |
| | | ccggcgccgtgcgtgcctacggaggtcggtcggtcctgcgctgcgcttgcgtgcagcagtccggcgcggcagcttggtgtcgtgcgcggttccgcctccagtctagcatggctctcccagg |
| | | ggccgccgtcgcgcgctctttatagcggccgggaagccggacgcgtaacctggtgtctggtggtgactgctccgtggctcggcgtgggcgttccggcaggcagcgggcccgtggcgtcag |
| | | ccgtgacgacgcgacgacgaccgagaagaggttgaccccccgcggtcgttgggtggactgatgactgatatcccctcgcgtcatcgccgtggcggtgagtgacgtcacc[C]AGGCT |
| | | GACGACTGGGTACCCACTCCACTTGTAGTGGCATAAGCACACTACTCTCTCTCTCTCTCTTTCTTTCAAATGTGTACTAATTG |
| | | AAATATAAGGGAGGAACC |
| 117 | 4449-01- | CTTGGATCCTGCGATCCGCCGCTGGAGCGTAACCACCGCGTCCACATGCTGCTTCATTGGTGCCGGCGCGGAGGCTG |
| | E12 | CTTAGCTCGCCGCCGAGGACTACTTTGCCCGCCACGGTT[ACGGTT]cctttcctaaaactgagcgttttcctttctttcgaccactacgcatttgaaa |
| | | attgaaatgcagatgtctaatttcccccttaccagaatggtgggcgtcatgagctacttccaccccccacatactacttcgcagttcgcactagcaatttgcctcagctcgtttttaaatataaa |
| | | aagtaataattgaattcaaatcgtggaagcagccaacatacttgaaatcggggggttggacggccaatcgggcaggaactcgtcgaggggatggcagtgcaggcggaggtagtcgcgccagt |
| | | tgtgcaccgtctccttgcgcacgttgaagctggtggacagccggatcttcctggcggggtcgtcggagtagagcttggccttctcctcggggggcagccggaagaagacgcgccccgcggc |
| | | catcaccgccgcgaccagggcggcgtgtatcccgtggttgacgacctggaagaagccgtgcgagcggcaggcgtcgccgacggggccacgaccgcgccgcggtcggggctgcccag |
| | | gtccaccacggggatgcgcgcgccggtcacgacctccgcgaggcgcgggcgctccggctccggccgcacgtagctccccggcagcgtgtcgtgcacggccgtcgacaggaggtgctcc |
| | | gccatgccgccggcgccgtgcgtgcctacggaggtcggtcggtcctgcgctgcgcttgcgtgcagcagtccggcgcggcagcttggtgtcgtgcgcggttccgcctccagtctagcatggc |
| | | tctcccaggggccgccgtcgcgcgctctttatagcggccgggaagccggacgcgtaacctggtgtctggtggtgactgctccgtggctcggcgtgggcgttccggcaggcagcgggcccg |
| | | tggcgtcagccgtgacgacgcgacgacgaccgagaagaggttgaccccccgcggtcgttgggtggactgatgactgatatcccctcgcgtcatcgccgtggggtgagtgacgtcaccCA |
| | | CTCCACTTGTAGTGGCATAAGCACACTACTCTCTCTCTCTCTCTTTCTTTCAAATGTGTACTAATTGAAATATAAGGGAGGAAC |
| | | C |
| 118 | 4449-01-F4 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTtttcctttcctaaaactgagcgttttcctttctttcgaccactacgcatttgaaaattgaaatgca |
| | | gatgtctaatttcccccttaccagaatggtgggcgtcatgagctacttccaccccccacatactacttcgcagttcgcactagcaatttgcctcagctcgtttttaaatataaaaagtaataattg |
| | | aattcaaatcgtggaagcagccaacatacttgaaatcggggggttggacggccaatcgggcaggaactcgtcgaggggatggcagtgcaggcggaggtagtcgcgccagttgtgcaccgtct |
| | | ccttgcgcacgttgaagctggtggacagccggatcttcctggggggtcgtcggagtagagcttggccttctcctcggggggcagccggaagaagacgcgccccgcggccatcaccgccg |
| | | cgaccagggcggcgtgtatcccgtggttgacgacctggaagaagccgtgcgagcggcaggcgtcgccgacggcggccacgaccgcgccgcggtcggggctgcccaggtccaccacg |
| | | gggatgcgcgcgccggtcacgacctccgcgaggcgcgggcgctccggctccggccgcacgtagctccccggcagcgtgtcgtgcacggccgtcgacaggaggtgctccgccatgccg |
| | | ccggcgccgtgcgtgcctacggaggtcggtcggtcctgcgctgcgcttgcgtgcagcagtccggcgcggcagcttggtgtcgtgcgcggttccgcctccagtctagcatggctctcccagg |
| | | ggccgccgtcgcgcgctctttatagcggccgggaagccggacgcgtaacctggtgtctggtggtgactgctccgtggctcggcgtgggcgttccggcaggcagcgggcccgtggcgtcag |
| | | ccgtgacgacgcgacgacgaccgagaagaggttgaccccccgcggtcgttgggtggactgatgactgatatcccctcgcgtcatcgccgtTGTAGTGGCATAAGCACACTA |
| | | CTCTCTCTCTCTCTCTTTCTTTCAAATGTGTACTAATTGAAATATAAGGGAGGAACC |
| 119 | 4449-01-F8 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTAaactgagcgttttcctttctttcgaccactacgcatttgaaaattgaaatgcagatgtctaatttcccccttaccagaatggtgggcgtc |
| | | atgagctacttccaccccccacatactacttcgcagttcgcactagcaatttgcctcagctcgtttttaaatataaaaagtaataattgaattcaaatcgtggaagcagccaacatacttgaaat |
| | | cgggcgggttggacggccaatcgggcaggaactcgtcgaggggatggcagtgcaggcggaggtagtcgcgccagttgtgcaccgtctccttgcgcacgttgaagctggtggacagccggatct |
| | | tcctggcggggtcgtcggagtagagcttggccttctcctcggggggcagccggaagaagacgcgccccgcggccatcaccgccgcgaccagggcggcgtgtatcccgtggttgacgacc |
| | | tggaagaagccgtgcgagcggcaggcgtcgccgacggcggccacgaccgcgccgcggtcggggctgcccaggtccaccacggggatgcgcgcgcggtcacgacctccgcgaggc |
| | | gcgggcgctccggctccggccgcacgtagctccccggcagcgtgtcgtgcacggccgtcgacaggaggtgctccgccatgccgccggcgccgtgcgtgcctacggaggtcggtcggtc |
| | | ctgcgctgcgcttgcgtgcagcagtccggcgcggcagcttggtgtcgtgcgcggttccgcctccagtctagcatggctctcccaggggccgccgtcgcgcgctctttatagcggccgggaag |
| | | ccggacgcgtaacctggtgtctggtggtgactgctccgtggctcggcgtgggcgttccggcaggcagcgggcccgtggcgtcagccgtgacgacgcgacgacgaccgagaagaggttga |
| | | ccccccgcggtcgttgggtggactgatgactgatatcccctcgcgtcatcgccgtggcggtgagtgacgACTGGGTACCCACTCCACTTGTAGTGGCATAAGCA |
| | | CACTACTCTCTCTCTCTCTCTTTCTTTCAAATGTGTACTAATTGAAATATAAGGGAGGAACC |
| 120 | 4449-01- | CTTGGATCCTGCGATCCGCCGCTGGAGCGTAACCACCGCGTCCACATGCTGCTTCATTGGTGCCGGCGCGGAGGCTG |
| | G10 | CTTAGCTCGCCGCCGAGGACTACtcctaaaactgagcgttttcctttctttcgaccactacgcatttgaaaattgaaatgcagatgtctaatttcccccttaccagaatggt |
| | | gggcgtcatgagctacttccaccccccacatactacttcgcagttcgcactagcaatttgcctcagctcgtttttaaatataaaaagtaataattgaattcaaatcgtggaagcagccaacatact |
| | | tgaaatcgggcgggttggacggccaatcgggcaggaactcgtcgaggggatggcagtgcaggcggaggtagtcgcgccagttgtgcaccgtctccttgcgcacgttgaagctggtggacagc |
| | | cggatcttcctggcggggtcgtcggagtagagcttggccttctcctcggggggcagccggaagaagacgcgccccgcggccatcaccgccgcgaccagggcggcgtgtatcccgtggttg |
| | | acgacctggaagaagccgtgcgagcggcaggcgtcgccgacggcggccacgaccgcgccgcggtcggggctgcccaggtccaccacggggatgcgcgcgccggtcacgacctccgc |
| | | gaggcgcgggcgctccggctccggccgcacgtagctccccggcagcgtgtcgtgcacggccgtcgacaggaggtgctccgccatgccgccggcgccgtgcgtgcctacggaggtcggt |
| | | cggtcctgcgctgcgcttgcgtgcagcagtccggcgcggcagcttggtgtcgtgcgcggttccgcctccagtctagcatggctctcccaggggccgccgtcgcgcgctctttatagcggccg |
| | | ggaagccggacgcgtaacctggtgtctggtggtgactgctccgtggctcggcgtgggcgttccggcaggcagcgggcccgtggcgtcagccgtgacgacgcgacgacgaccgagaaga |
| | | ggttgaccccccgcggtcgttgggtggactgatgactgatatcccctcgcgtcatcgccgtggcggtgagtgacgtcacACGACTGGGTACCCACTCCACTTGTAGTG |
| | | GCATAAGCACACTACTCTCTCTCTCTCTCTTTCTTTCAAATGTGTACTAATTGAAATATAAGGGAGGAACC |
| 121 | 4449-01-H2 | CTTGGATCCTGCGATCCGCCGCTGGAGCGTAACCACCGCGTCCACATGCTGCTTCATTGGTGCCGGCGCGGAGGCTG |
| | | CTTAGCTCGCCGCCGAGGACTACTTTGCCCGCCACGGTTACGtttcctttcctaaaactgagcgttttcctttctttcgaccactacgcatttgaaaattga |
| | | aatgcagatgtctaatttcccccttaccagaatgatgagctacttccaccccccacatactacttegcagttegcactagcaatttgcctcagctcgtttttaaatataaaaagtaata |
| | | attgaattcaaatcgtggaagcagccaacatacttgaaatcggggggttggacggccaatcgggcaggaactcgtcgaggggatggcagtgcaggcggaggtagtcgcgccagttgtgca |
| | | ccgtctccttgcgcacgttgaagctggtggacagccggatcttcctggcggggtcgtcggagtagagcttggccttctcctcggggggcagccggaagaagacgcgccccgcggccatcac |
| | | cgccgcgaccagggggcgtgtatcccgtggttgacgacctggaagaagccgtgcgagcggcaggcgtcgccgacggcggccacgaccgcgccgcggtcggggctgcccaggtcca |
| | | ccacggggatgcgcgcgccggtcacgacctccgcgaggcgcgggcgctccggctccggccgcacgtagctccccggcagcgtgtcgtgcacggccgtcgacaggaggtgctccgccat |
| | | gccgccggcgccgtgcgtgcctacggaggtcggtcggtcctgcgctgcgcttgcgtgcagcagtccggcgcggcagcttggtgtcgtgcgcggttccgcctccagtctagcatggctctccc |
| | | aggggccgccgtcgcgcgctctttatagcggccgggaagccggacgcgtaacctggtgtctggtggtgactgctccgtggctcggcgtgggcgttccggcaggcagcgggcccgtggcgt |
| | | cagccgtgacgacgcgacgacgaccgagaagaggttgaccccccgcggtcgttgggtggactgatgactgatatcccctcgcgtcatcgccgtggcggtgagtgacgtcCGACTGG |
| | | GTACCCACTCCACTTGTAGTGGCATAAGCACACTACTCTCTCTCTCTCTCTTTCTTTCAAATGTGTACTAATTGAAATATAAGG |
| | | GAGGAACC |
| 122 | 4449-01- | CTTGGATCCTGCGATCCGCCGCTGGAGCGTAACCACCGCGTCCACATGCTGCTTCATTGGTGCCGGCGCGGAGGCTG |
| | H11-1 | CTTAGCTCGCCGCCGAGGACTACTTTGCCCGCCACGGTTatcgtttcctttcctaaaactgagcgttttcctttctttcgaccactacgcatttgaaaattga |
| | | aatgcagatgtctaatttcccccttaccagaatggtgggcgtcatgagctacttccaccccccacatactacttcgcagttcgcactagcaatttgcctcagctcgtttttaaatataaaaagta |
| | | ataattgaattcaaatcgtggaagcagccaacatacttgaaatcggggggttggacggccaatcgggcaggaactcgtcgaggggatggcagtgcaggcggaggtagtcgcgccagttgtgca |
| | | ccgtctccttgcgcacgttgaagctggtggacagccggatcttcctggcggggtcgtcggagtagagcttggccttctcctcggggggcagccggaagaagacgcgccccgcggccatcac |
| | | cgccgcgaccagggcggcgtgtatcccgtggttgacgacctggaagaagccgtgcgagcggcaggcgtcgccgacggcggccacgaccgcgccgcggtcggggctgcccaggtcca |
| | | ccacggggatgcgcgcgccggtcacgacctccgcgaggcgcgggcgctccggctccggccgcacgtagctccccggcagcgtgtcgtgcacggccgtcgacaggaggtgctccgccat |
| | | gccgccggcgccgtgcgtgcctacggaggtcggtcggtcctgcgctgcgcttgcgtgcagcagtccggcgcggcagcttggtgtcgtgcgcggttccgcctccagtctagcatggctctccc |
| | | aggggccgccgtcgcgcgctctttatagcggccgggaagccggacgcgtaacctggtgtctggtggtgactgctccgtggctcggcgtgggcgttccggcaggcagcgggcccgtggcgt |
| | | cagccgtgacgacgcgacgacgaccgagaagaggttgaccccccgcggtcgttgggtggactgatgactgatatcccctcgcgtcatcgccgtggcggtgagtgacgACTGGGTAC |
| | | CCACTCCACTTGTAGTGGCATAAGCACACTACTCTCTCTCTCTCTCTTTCTTTCAAATGTGTACTAATTGAAATATAAGGGAGG |
| | | AACC |
| 123 | 4449-01- | CTTGGATCCTGCGATCCGCCGCTGGAGCGTAACCACCGCGTCCACATGCTGCTTCATTGGTGCCGGCGCGGAGGCTG |
| | H11-5 | CTTAGCTCGCCGCCGAGGACTACTTTGCCCGCCACGGTTACGGAcctttcctaaaactgagcgttttcctttctttcgaccactacgcatttgaaaatt |
| | | gaaatgcagatgtctaatttcccccttaccagaatggtgggcgtcatgagctacttccaccccccacatactacttcgcagttcgcactagcaatttgcctcagctcgtttttaaatataaaaag |
| | | taataattgaattcaaatcgtggaagcagccaacatacttgaaatcggggggttggacggccaatcgggcaggaactcgtcgaggggatggcagtgcaggcggaggtagtcgcgccagttgt |
| | | gcaccgtctccttgcgcacgttgaagctggtggacagccggatcttcctggggggtcgtcggagtagagcttggccttctcctcggggggcagccggaagaagacgcgccccgcggccat |
| | | caccgccgcgaccagggcggcgtgtatcccgtggttgacgacctggaagaagccgtgcgagcggcaggcgtcgccgacggcggccacgaccgcgccgcggtcggggctgcccaggt |
| | | ccaccacggggatgcgcgcgccggtcacgacctccgcgaggcgcgggcgctccggctccggccgcacgtagctccccggcagcgtgtcgtgcacggccgtcgacaggaggtgctccg |
| | | ccatgccgccggcgccgtgcgtgcctacggaggtcggtcggtcctgcgctgcgcttgcgtgcagcagtccggcgcggcagcttggtgtcgtgcgcggttccgcctccagtctagcatggctc |
| | | tcccaggggccgccgtcgcgcgctctttatagcggccgggaagccggacgcgtaacctggtgtctggtggtgactgctccgtggctcggcgtgggcgttccggcaggcagcgggcccgtg |
| | | gcgtcagccgtgacgacgcgacgacgaccgagaagaggttgaccccccgcggtcgttgggtggactgatgactgatatcccctcgcgtcatcgccgtggcggtgagtgacgtcaccaccc |
| | | ACGACTGGGTACCCACTCCACTTGTAGTGGCATAAGCACACTACTCTCTCTCTCTCTCTTTCTTTCAAATGTGTACTAATTGAA |
| | | ATATAAGGGAGGAACC |
| 124 | 4449-01-H12 | CTTGGATCCTGCGATCCGCCGCTGGAGCGTAACCACCGCGTCCACATGCTGCTTCATTGGTGCCGGCGCGGAGGCTG |
| | | CTTAGCTCGCCGatcgtttcctttcctaaaactgagcgttttcctttctttcgaccactacgcatttgaaaattgaaatgcagatgtctaatttcccccttaccagaatggtgggcgtca |
| | | tgagctacttccaccccccacatactacttcgcagttcgcactagcaatttgcctcagctcgtttttaaatataaaaagtaataattgaattcaaatcgtggaagcagccaacatacttgaaatc |
| | | gggcgggttggacggccaatcgggcaggaactcgtcgaggggatggcagtgcaggcggaggtagtcgcgccagttgtgcaccgtctccttgcgcacgttgaagctggtggacagccggatcttc |
| | | ctggcggggtcgtcggagtagagcttggccttctcctcggggggcagccggaagaagacgcgccccgcggccatcaccgccgcgaccagggcggcgtgtatcccgtggttgacgacctg |
| | | gaagaagccgtgcgagcggcaggcgtcgccgacggcggccacgaccgcgccgcggtcggggctgcccaggtccaccacggggatgcgcgcgccggtcacgacctccgcgaggcgc |
| | | gggcgctccggctccggccgcacgtagctccccggcagcgtgtgtgcacggccgtcgacaggaggtgctccgccatgccgccggcgccgtgcgtgcctacggaggtcggtcggtcctg |
| | | cgctgcgcttgcgtgcagcagtccggcgcggcagcttggtgtcgtgcgcggttccgcctccagtctagcatggctctcccaggggccgccgtcgcgcgctctttatagcggccgggaagcc |
| | | ggacgcgtaacctggtgtctggtggtgactgctccgtggctcggcgtgggcgttccggcaggcagcgggcccgtggcgtcagccgtgacgacgcgacgacgaccgagaagaggttgacc |
| | | ccccgcggtcgttgggtggactgatgactgatatcccctcgcgtcatcgccgtggcggtgagtgacgtcaccacACGACTGGGTACCCACTCCACTTGTAGTGGCA |
| | | TAAGCACACTACTCTCTCTCTCTCTCTTTCTTTCAAATGTGTACTAATTGAAATATAAGGGAGGAACC |

### Example 2. Dissecting the Molecular Mechanism(s) Conferring the High Inversion Rate Induced by LbCas12a-Trex2 Fusion

This example is to examine the necessity of Trex2 dimerization and/or 3′-5′ exonuclease activity in inducing the high inversion rate by LbCas12a-Trex2 fusion. A catalytic-deficient Trex2 mutant (Trex2^{CD}, SEQ ID NO:18), a dimerization-deficient Trex2 mutant (Trex2^{DD}, SEQ ID NO:19), and a single-chained Trex2 homodimer (scTrex2, SEQ ID NO:20) were fused to the C-terminus of LbCas12a, respectively, and the inversion-inducing efficacy of each fused variant will be compared with that of the original LbCas12a-Trex2 fusion. Based on previous studies (Chen et al., Nucleic Acids Res. 2007 April; 35 (8): 2682-2694.; Delacote et al., PLOS One. 2013; 8 (1): e53217.), Trex2^{CD} loses the exonuclease activity but retains the capability of forming homodimers; Trex2^{DD} is impaired in forming homodimers and thus also loses the exonuclease activity; scTrex2 is capable of forming an intramolecular dimer after translation, which exhibits exonuclease activity but is no longer capable of mediating dimerization between two fusion proteins.

Construct 27431 that expresses LbCas12a-Trex2^{CD} was generated by mutating the codons encoding amino acid residues H188 and D193 in construct 25962 to alanine-encoding codons. Construct 27432 that expresses LbCas12a-Trex2^{DD} was generated by mutating the codons encoding amino acid residues E29, K59, N94, R107, and E191 in 25962 to alanine-encoding codons. Construct 27433 that expresses LbCas12a-scTrex2 was generated by inserting the DNA sequence encoding a polypeptide (TPPQTGLDVPY) linker and a second, re-coded Trex2 monomer right upstream to the C-terminal NLS (SEQ ID NO:21). All constructs use the same paired gRNAs targeting ZmDMR6.

TABLE 6
Sequences used in Example 2.

| SEQ | | | |
|---|---|---|---|
| ID NO. | Description | Type | Source |
| 18 | Catalytic-deficient mouse Trex2 | Protein | Mus |
| | (Trex2^{CD}), monomer | | musculus |
| 19 | Dimerization-deficient mouse Trex2 | Protein | Mus |
| | (Trex2^{DD}), monomer | | musculus |
| 20 | Single-chained Trex2 homodimer | Protein | Synthetic |
| | (scTrex2) | | |
| 21 | Single-chained Trex2 homodimer | DNA | Synthetic |
| | (scTrex2), coding sequence | | |

TABLE 7
DNA constructs used in Example 2.

| Construct | | | | | Transfor- |
|---|---|---|---|---|---|
| # | D156R | Exo fusion | gRNA1 | gRNA2 | mation |
| 25962 | + | C-terminal | ZmDMR6- | ZmDMR6- | Agro |
| | | Trex2 | crRNA1 | crRNA2 | LBA4404 |
| 27431 | + | C-terminal | ZmDMR6- | ZmDMR6- | Agro |
| | | Trex2^{CD} | crRNA1 | crRNA2 | LBA4404 |
| 27432 | + | C-terminal | ZmDMR6- | ZmDMR6- | Agro |
| | | Trex2^{DD} | crRNA1 | crRNA2 | LBA4404 |
| 27433 | + | C-terminal | ZmDMR6- | ZmDMR6- | Agro |
| | | scTrex2 | crRNA1 | crRNA2 | LBA4404 |

Transgenic plants were generated and analyzed as described in Example 1. The efficiency of targeted inversion of each construct will be compared with that of construct 25962 and that of 26297, summarized in Table 8. With the two catalytic residues in Trex2 mutated, the LbCas12a-Trex2^{CD} fusion can still induce as high frequency of fragment inversion as the original LbCas12a-Trex2 fusion does, if not higher. With the five dimerization-mediating residues mutated, the inversion rate induced by the LbCas12a-Trex^{DD} fusion drops to only one third of that induced by the original LbCas12a-Trex2 fusion. Part of the editing outcomes by the LbCas12a-scTrex2 is unexpected—the editing efficiency at the gRNA1 target drops to around one third of the efficiency at the same target by other constructs. However, among the 13 plants in which both targets were edited, the targeted inversion was identified in one plant, at a frequency (7.7%) comparable to that of 27432 (6.0%). Together, these results suggest that the dimerization capability of Trex2, rather than the 3′-5′ exonuclease activity, is required for the high inversion rate induced by the LbCas12a-Trex2 fusion.

TABLE 8
Summary of editing outcomes.

| | Cas12a | 3′-5′ | | | % | % | |
|---|---|---|---|---|---|---|---|
| | Endo. | Exo. | Dimerization | # Of | Editing | Editing | 1 kb |
| Construct # | Activity | Activity | Capability | Cas12a+ | (Grna1) | (Grna2) | Inversion |
| 25962 | + | + | + | 152 | 74.3% | 82.9% | 28 (18.4%) |
| 26297 | + | − | − | 75 | 81.3% | 88.0% | 1 (1.3%) |
| 27431 | + | − | + | 61 | 82.0% | 86.9% | 14 (23.0%) |
| 27432 | + | − | − | 50 | 80.0% | 86.0% | 3 (6.0%) |
| 27433 | + | + | − | 49 | 26.5% | 89.8% | 1 (2.0%)* |

*Underestimated due to unexpected low editing efficiency at the gRNA1 target.

### Example 3. Use LbCas12a-Trex2 Fusion to Increase the Frequency of Fragment Excision Between Paired Guide RNA Targeting Sites in Maize Genome

This example is to demonstrate that the LbCas12a-Trex2 fusion, in conjunction with a pair of gRNAs targeting the same chromosome with binding sites on opposite strands and both outside of a target region (e.g., as shown in FIG. 2), is able to significantly increase the frequency of excision of the target region.

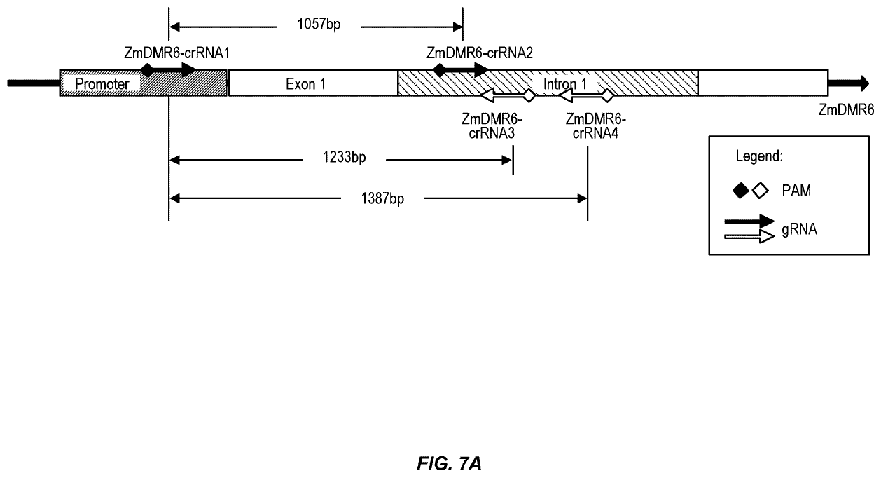

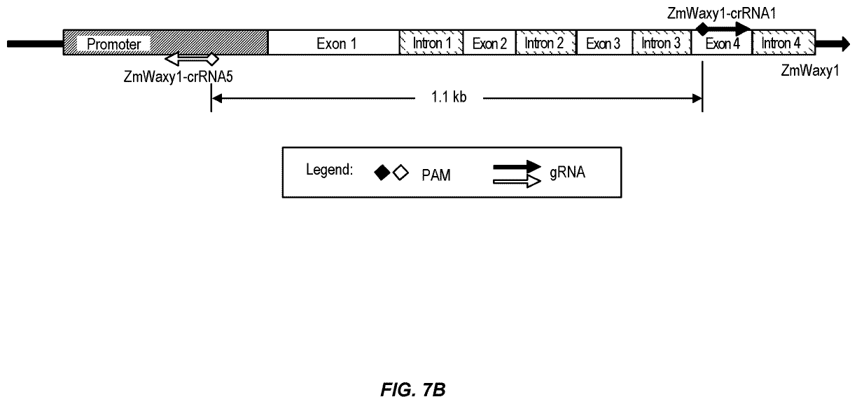

In Example 1, two gRNAs in the same orientation were selected to excise the first exon of ZmDMR6 gene. The frequency of desired excision were moderately increased when LbCas12a-Trex2 fusion was used in conjunction with the two gRNAs, compared with the non-fusion control (see Table 4). Based on the dimerization hypothesis described in Example 2, it was predicted that two gRNAs with binding sites on opposite strands outside of the target region may further increase the excision frequency induced by LbCas12a-Trex2. To design such gRNA pairs, ZmDMR6-crRNA1 was kept and paired with two gRNAs, ZmDMR6-crRNA3 (SEQ ID NO: 23) and ZmDMR6-crRNA4 (SEQ ID NO:24) respectively, of which the targets are downstream to that of ZmDMR6-crRNA2 and on the complementary strand (FIG. 7A).

TABLE 9
Sequences used in Example 3.

| SEQ | | | |
|---|---|---|---|
| ID NO. | Description | Type | Source |
| 23 | ZmDMR6 crRNA3 | RNA | Synthetic |
| 24 | ZmDMR6 crRNA4 | RNA | Synthetic |
| 25 | ZmWaxy1 crRNA1 | RNA | Synthetic |
| 26 | ZmWaxy1 crRNA5 | RNA | Synthetic |

The coding sequence of ZmDMR6-crRNA2 in construct 25962 was replaced by the coding sequence of ZmDMR6-crRNA3 and ZmDMR6-crRNA4, respectively, to generate fusion constructs 26710 and 26711. The coding sequence of ZmDMR6-crRNA2 in construct 26297 was replaced by the coding sequence of ZmDMR6-crRNA3 and ZmDMR6-crRNA4, respectively, to generate non-fusion control constructs 26712 and 26713.

TABLE 10
DNA constructs used in Example 3.

| Construct | | | | | |
|---|---|---|---|---|---|
| # | D156R | Exo fusion | gRNA1 | gRNA2 | Transformation |
| 26710 | + | C-terminal | ZmDMR6-crRNA1 | ZmDMR6-crRNA3 | Agro LBA4404 |
| | | Trex2 | (targeting promoter, | (targeting intron1, on | |
| | | | on coding strand) | complementary strand) | |
| 26711 | + | C-terminal | ZmDMR6-crRNA1 | ZmDMR6-crRNA4 | Agro LBA4404 |
| | | Trex2 | | (targeting intron1, on | |
| | | | | complementary strand) | |
| 26712 | + | — | ZmDMR6-crRNA1 | ZmDMR6-crRNA3 | Agro LBA4404 |
| 26713 | + | — | ZmDMR6-crRNA1 | ZmDMR6-crRNA4 | Agro LBA4404 |
| 26958 | + | C-terminal | ZmWaxy1-crRNA1 | ZmWaxy1-crRNA5 | Agro LBA4404 |
| | | Trex2 | (targeting Exon 4, on | (targeting promoter, on | |
| | | | coding strand) | complementary strand) | |
| 26961 | + | — | ZmWaxy1-crRNA1 | ZmWaxy1-crRNA5 | Agro LBA4404 |

Transgenic plants were generated and analyzed as described in Example 1, except that the reverse primer for PCR was redesigned. The editing outcomes are summarized in Table 11. Interestingly, all four constructs resulted in extremely low to zero inversion frequencies. The excision frequencies resulted from all four constructs are below 10%, though the presence of Trex2 nearly doubled the excision frequency between ZmDMR6-crRNA1 and ZmDMR6-crRNA4. The other binding sites designed (in Table 1) that were expected to significantly improve the excision frequency between the two target sites were also tested.

TABLE 11
Summary of editing outcomes.

| Con- | # of | % | % | | | |
|---|---|---|---|---|---|---|
| struct | transgenic | Editing | Editing | 1 kb | | 1 kb |
| # | (Cas12a+) | (gRNA1) | (gRNA2) | excision | | inversion |
| 26710 | 114 | 64.9% | 87.7% | 6 | (5.3%) | 1 (0.9%) |
| 26711 | 109 | 78.9% | 89.9% | 6 | (5.5%) | 0 |
| 26712 | 105 | 76.2% | 83.8% | 7 | (6.7%) | 0 |
| 26713 | 105 | 73.3% | 89.5% | 3 | (2.9%) | 0 |
| 26958 | 70 | 82.3% | 81.5% | 41 | (58.6%) | 8 (11.4%) |
| 26961 | 72 | 88.3% | 80.8% | 15 | (20.8%) | 4 (5.6%) |

The ZmWaxy1 gene was chosen for testing the prediction that two gRNAs with binding sites on opposite strands inside of the target region may significantly increase the excision frequency induced by LbCas12a-Trex2. ZmWaxy1-crRNA1 (SEQ ID NO:25) was designed to target a sequence adjacent to a TTTG PAM in Exon 4 on the coding strand, while ZmWaxy1-crRNA5 (SEQ ID NO:26) was designed to target a sequence adjacent to a TTTA PAM in the promoter region on the complementary strand (FIG. 7B).

The coding sequences of ZmDMR6-crRNA1 and ZmDMR6-crRNA2 in construct 25962 was replaced by the coding sequences of ZmWaxy1-crRNA1 and ZmWaxy1-crRNA5, respectively, to generate fusion construct 26958. The coding sequences of ZmDMR6-crRNA1 and ZmDMR6-crRNA2 in construct 26297 was replaced by the coding sequences of ZmWaxy1-crRNA1 and ZmWaxy1-crRNA5, respectively, to generate non-fusion control construct 26961. Transgenic plants were generated and analyzed similarly as described in Example 1, and the editing outcomes are also summarized in Table 9. The gRNA pair resulted in high (˜20%) excision frequency even without Trex2; however the addition of Trex2 nearly tripled the excision frequency to a striking 58.6%. The results of the excision experiments in this example are not consistent with the dimerization hypothesis but can be explained by an alternative hypothesis based on repair inhibition. For example, the presence of Trex2 may inhibit immediate repair by canonical NHEJ between the two ends at one DSB site (likely due to the exonuclease activity), but leaves two unbound ends at two DSB sites open for NHEJ repair. An NHEJ repair between the two unbound ends may then result in preferred repair outcomes, depending on the orientation of the gRNA target sites.

### Example 4. Use LbCas12a-Trex2 Fusion to Increase the Frequencies of Fragment Inversion and Excision Between Paired Guide RNA Targeting Sites in Soybean Genome

This example is to demonstrate the efficacy of LbCas12a-Trex2 fusion in increasing the frequencies of fragment inversion and excision in a dicot crop, soybean. The soybean FATTY ACID DESATURASE 2A (FAD2-1A) gene (SEQ ID NO:27) was selected for the experiments.

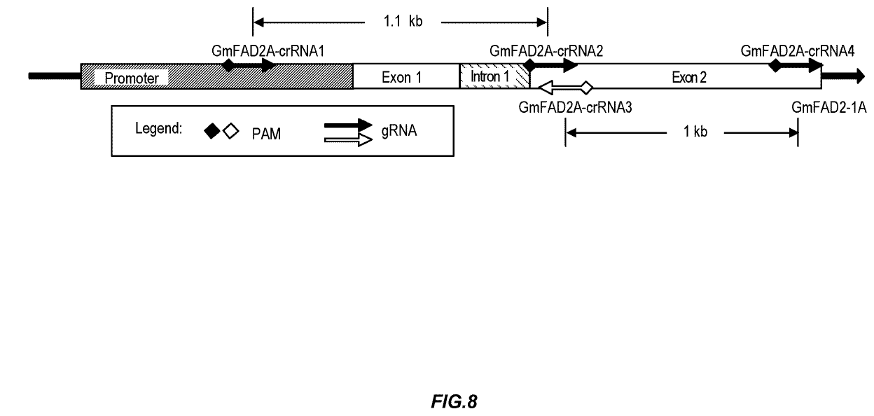

The first gRNA, GmFAD2A-crRNA1 (SEQ ID NO:28) was designed to target a sequence adjacent to a TTTG PAM in the FAD2-1A promoter region on the coding strand. A second gRNA, GmFAD2A-crRNA2 (SEQ ID NO:29) was designed to target a sequence adjacent to a TTTG PAM next to the 3′ splicing site of the first intron on the coding strand. The distance between crRNA1 and crRNA2 target sites is about 1.1 kb. A third gRNA, GmFAD2A-crRNA3 (SEQ ID NO:30) was designed to target a sequence adjacent to a TTTG in the second exon on the complementary strand. A fourth gRNA, GmFAD2A-crRNA4 (SEQ ID NO:31) was designed to target a sequence adjacent to a TTTG in the second exon on the coding strand. The distance between crRNA3 and crRNA4 target sites is about 1 kb (FIG. 8).

The following outcome predictions are based on the maize results described in Examples 1 and 3. GmFAD2A-crRNA1 and crRNA2 are in the same orientation, therefore when both gRNAs are co-expressed with LbCas12a-Trex2 fusion in soybean cells, they are expected to induce significantly higher frequency of inverting the fragment between the two gRNA target sites, compared with being co-expressed with LbCas12a. GmFAD2A-crRNA3 and crRNA4 bind to opposite strands inside of the target region, therefore when both gRNAs are co-expressed with LbCas12a-Trex2 fusion in soybean cells, they are expected to induce significantly higher frequency of excising the fragment between the two gRNA target sites, compared with being co-expressed with LbCas12a.

The binary vectors constructed to test the efficacies of the Cas12a-exonuclease fusions in soybean are summarized in Table 12. The Arabidopsis codon-optimized coding sequence of LbCas12a-Trex2 fusion (SEQ ID NO:32), or the Arabidopsis codon-optimized coding sequence of LbCas12a (SEQ ID NO:33) was operably linked to a Figwort mosaic virus (FMV) enhancer (SEQ ID NO:34), an promoter of the Arabidopsis EF1A elongation factor gene (SEQ ID NO:35) and an Agrobacterium nopaline synthase terminator (SEQ ID NO:10), for constitutive expression in soybean cells. The two paired gRNAs in a tandem array and flanked by hammerhead (HH) and hepatitis delta virus (HDV) ribozymes (SEQ ID NOs: 36 and 37, respectively), were expressed under a soybean ubiquitin promoter (SEQ ID NO:38). The transcript containing the gRNA array was processed into separate mature crRNAs by the self-cleavage of ribozymes and the intrinsic RNase activity of LbCas12a. An aminoglycoside 3′-adenyltransferase gene (SEQ ID NO: 39), operably linked to a promoter of soybean EF1 elongation factor gene (SEQ ID NO:40) and a terminator of the pea (Pisum sativum) rib-1,5-bisphospate carboxylase (rbcS2) small subunit E9 gene (SEQ ID NO:41), confers resistance to antibiotic spectinomycin in plant cells and served as plant selection marker in all vectors.

TABLE 12
Sequences used in Example 4.

| SEQ | | | |
|---|---|---|---|
| ID NO. | Description | Type | Source |
| 27 | Soybean FAD2-1A gene, full length | DNA | Glycine max |
| 28 | GmFAD2A crRNA1 | RNA | Synthetic |
| 29 | GmFAD2A crRNA2 | RNA | Synthetic |
| 30 | GmFAD2A crRNA3 | RNA | Synthetic |
| 31 | GmFAD2A crRNA4 | RNA | Synthetic |
| 32 | LbCas12a-Trex2 coding sequence, | DNA | Synthetic |
| | Arabidopsis codon-optimized | | |
| 33 | LbCas12a coding sequence, | DNA | Synthetic |
| | Arabidopsis codon-optimized | | |
| 34 | Figwort mosaic virus (FMV) enhancer | DNA | Figwort |
| | | | mosaic virus |
| 35 | Arabidopsis EF1A promoter | DNA | Arabidopsis |
| | | | thaliana |
| 36 | Hammerhead (HH) ribozyme | RNA | Synthetic |
| 37 | Hepatitis delta virus (HDV) ribozyme | RNA | Hepatitis delta |
| | | | virus |
| 38 | Soybean ubiquitin promoter | DNA | Glycine max |
| 39 | Aminoglycoside 3′-adenyltransferase | DNA | Escherichia |
| | gene, coding sequence | | coli |
| 40 | Soybean EF1 promoter | DNA | Glycine max |
| 41 | 3′-UTR of the pea rib-1,5-bisphospate | DNA | Pisum sativum |
| | carboxylase (rbcS2) small subunit E9 | | |
| | gene | | |

TABLE 13
DNA constructs used in Example 4.

| Construct | | | | | |
|---|---|---|---|---|---|
| # | D156R | Exo fusion | gRNA1 | gRNA2 | Transformation |
| 27188 | + | C-terminal | GmFAD2A-crRNA1 | GmFAD2A-crRNA2 | Agro EHA101 |
| | | Trex2 | (targeting promoter, on | (targeting intron1, on | |
| | | | coding strand) | coding strand) | |
| 27253 | + | C-terminal | GmFAD2A-crRNA3 | GmFAD2A-crRNA4 | Agro EHA101 |
| | | Trex2 | | (targeting exon2, on | |
| | | | | complementary strand) | |
| 27194 | + | — | GmFAD2A-crRNA1 | GmFAD2A-crRNA2 | Agro EHA101 |
| 27254 | + | — | GmFAD2A-crRNA3 | GmFAD2A-crRNA4 | Agro EHA101 |

Each construct was delivered into the stem cells in the cotyledons of the Syngenta proprietary soybean elite line 06 KG by Agrobacterium tumefaciens-mediated transformation. The transformed cotyledons were subject to spectinomycin selection and plantlets will be regenerated through tissue culture procedures. The regenerated plantlets were sampled for DNA extraction, among which the transgenic plants harboring the constructs were identified by TaqMan assays.

PCRs were used to detect the inversion and excision events in the samples. For 27188 and 27194, a forward primer upstream to GmFAD2A-crRNA1 target site and a reverse primer downstream to GmFAD2A-crRNA2 target site were designed to amplify the sequences around and between the two target sites. A second “forward primer” (priming to the same strand as the first forward primer does) between GmFAD2A-crRNA1 and GmFAD2A-crRNA2 target sites was designed to amplify inversed fragment when paired with the first forward primer. For 27253 and 27254, a forward primer upstream to GmFAD2A-crRNA3 target site and a reverse primer downstream to GmFAD2A-crRNA4 target site were designed to amplify the sequences around and between the two target sites, and fragment excision between the two target sites will result in a smaller PCR amplicon. The results based on agrose gel electrophoresis of the PCR products are summarized in Table 14. The results corroborate the findings in maize: When the two paired target sites are on same strand, one within target region and one outside target region, the editing outcomes strongly prefer fragment inversion; when the two paired target sites are on opposite strands and both are within target region, the editing outcomes prefer fragment excision.

TABLE 14
Summary of editing outcomes.

| | # of transgenic | | | |
|---|---|---|---|---|
| Construct # | (Cas12a+) | Excision | | Inversion |
| 27188 | 80 | 3 | (3.8%) | 45 (56.3%) |
| 27194 | 59 | 16 | (27.1%) | 9 (15.3%) |
| 27253 | 65 | 33 | (50.8%) | 17 (26.2%) |
| 27254 | 65 | 26 | (40%) | 17 (26.2%) |

Due to the high degree of mosaicism often seen in the T₀ plants generated by organogenesis-based transformation, the proportion of desired mutations will be determined by NGS and the inheretibility of desired mutations will be determined in T₁ population.

### Example 5. Use Cas12a-Trex2 Fusion to Achieve Efficient Targeted Insertion Via NHEJ/MMEJ Repair in Maize

This example is to demonstrate LbCas12a-Trex2 fusion, in conjunction with gRNAs in carefully designed orientations, may efficiently mediate the insertion of a 3.6-kb expression cassette of the 5-enolpyruvylshikimate-3-phosphate synthase gene from Agrobacterium tumefaciens strain CP4 (CP4 EPSPS; the cargo sequence, SEQ ID NO:42) into a targeted gap in the maize genome, via the NHEJ/MMEJ repair pathway (i.e. without the need of long homology in donor DNA).

The gRNA pair ZmDMR6-crRNA1 and ZmDMR6-crRNA3 (see Example 3), which have binding sites on opposite strands outside of a target region (e.g., as shown in FIG. 3A), was selected to generate a gap between the two target sites in the ZmDMR6 gene. The target sequence of crRNA1 and crRNA2 (SEQ ID NOs: 43 and 44, respectively) were added to the 5′- and 3′-end of the cargo sequence, respectively, in such a way that the crRNA binding sites are on opposite strands but within the donor nucleotide region (e.g., as shown in FIG. 3A), resulting in the cargo-containing donor DNA. The donor DNA was cloned in a 1.8-kb miniature pUC57 backbone, resulting in the donor construct 27022.

TABLE 15
Sequences used in Example 5.

| SEQ | | | |
|---|---|---|---|
| ID. | Description | Type | Source |
| 42 | CP4 EPSPS expression cassette, | DNA | Synthetic |
| | driven by rice actin1 promoter | | |
| 43 | ZmDMR6-crRNA1 target sequence, | DNA | Zea mays |
| | PAM included | | |
| 44 | ZmDMR6-crRNA3 target sequence, | DNA | Zea mays |
| | PAM included | | |

TABLE 16
DNA constructs used in Example 5.

| Construct | | |
|---|---|---|
| # | Description | Transformation |
| 26710 | As described in Example 3. Express | Biolistic |
| | LbCas12a-Trex2 fusion and a gRNA pair to | |
| | generate a gap in ZmDMR6 gene. | |
| 26712 | As described in Example 3. The non-fusion | Biolistic |
| | control of construct 26710. | |
| 27022 | Donor construct harboring a CP4 EPSPS | Biolistic |
| | expression cassette, flanked by the | |
| | gRNA target sequences. | |

Linearized plasmid DNA of constructs 26710 and 27022 will be co-delivered into maize immature embryos by biolistic transformation. The bombarded embryos will be subject to mannose selection and plantlets will be regenerated through tissue culture. The regenerated plantlets will be sampled for DNA extraction, among which the transgenic plants harboring the constructs will be identified by TaqMan assays. As a non-fusion control, transgenic plants co-transformed with constructs 26712 and 27022 will be generated following the same procedures.

Multiple junction PCRs will be designed to identify the insertion at the target locus, each using a genomic-specific primer and a cargo-specific primer. The amplicons of junction PCRs will be Sanger sequenced to characterize the junction sequences. A long PCR using two genomic-specific primers flanking the insertion site will be performed to validate the length of the insertion. Since the cargo is inserted via NHEJ/MMEJ repair, it is anticipated that the cargo can be inserted in either direction, and small indels can be found at the junctions.

### Example 6. Use Cas12a-Trex2 Fusion to Achieve Efficient Targeted Insertion Via Homology-Directed Repair (HDR) in Maize

This example is to demonstrate LbCas12a-Trex2 fusion, in conjunction with gRNAs in carefully designed orientations, may efficiently mediate the insertion of a 3.8-kb CP4 EPSPS expression cassette (cargo sequence, SEQ ID NO:45) into a targeted gap in the maize genome, via the HDR pathway (i.e. with long homology sequences flanking the cargo sequence). An intergenic region on maize chromosome 1, identified as ZmSH1, was selected to demonstrate this design.

One gRNA, ZmSH1-crRNA1, was designed to target a sequence (SEQ ID NO:46) adjacent to a TTTG PAM on the positive strand. Another gRNA, ZmSH1-crRNA2, was designed to target a sequence (SEQ ID NO:47) adjacent to a TTTG PAM on the negative strand. The two gRNA target sites are 167 bp apart in the AX5707 genome, and they are on opposite strands outside the target region, leading to generation of a gap in ZmSH1 when co-expressed with LbCas12a-Trex2 fusion or non-fusion LbCas12a.

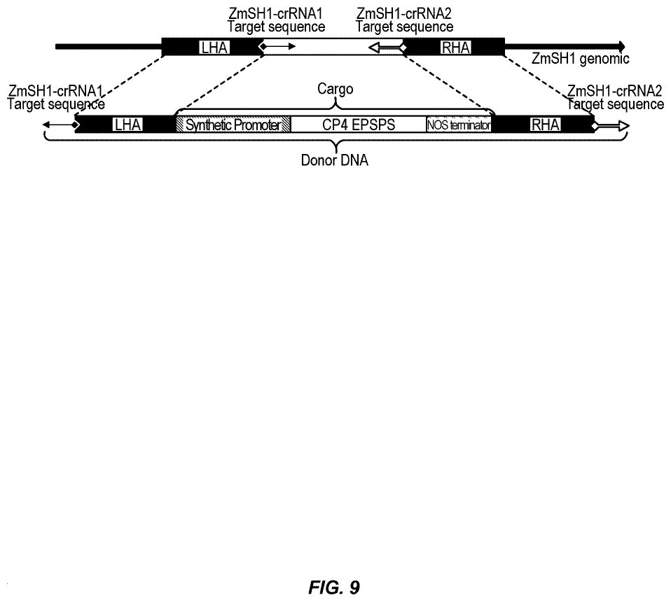

A 458-bp genomic sequence (SEQ ID NO:48) upstream to the PAM of ZmSH1-crRNA1 target was selected as the left homology arm (LHA) to mediate HDR, and was added to the 5′ end of the cargo sequence. A 509-bp genomic sequence (SEQ ID NO:49) downstream to the PAM of ZmSH1-crRNA2 target was selected as the right homology arm (RHA), and was added to the 3′ end of the cargo sequence. The target sequences of ZmSH1-crRNA1 and ZmSH1-crRNA2, both with a TTTG PAM added to the 5′ end, were added to the 5′ end of the left homology arm and the 3′ end of the right homology arm, respectively and in such a way that the crRNA binding sites are on opposite strands but within the donor nucleotide region, to generate the donor DNA (FIG. 9).

TABLE 17
Sequences used in Example 6.

| SEQ | | | |
|---|---|---|---|
| ID NO. | Description | Type | Source |
| 45 | CP4 EPSPS expression cassette, driven | DNA | Synthetic |
| | by a synthetic promoter | | |
| 46 | ZmSH1-crRNA1 target sequence | DNA | Zea mays |
| 47 | ZmSH1-crRNA2 target sequence | DNA | Zea mays |
| 48 | Left homology arm | DNA | Zea mays |
| 49 | Right homology arm | DNA | Zea mays |

The donor DNA, the Cas12a-Trex2 expression cassette, the gRNA expression cassette, and a PMI selection marker, will be built in a single T-DNA region in a binary vector. The resulting construct will be used to transform immature maize embryos, and transgenic plants will be generated through tissue culture with mannose selection and will be identified by TaqMan assays.

Multiple junction PCRs will be designed to identify the desired insertion at the target locus, each using a genomic-specific primer and a cargo-specific primer. The amplicons of junction PCRs will be Sanger sequenced to characterize the junction sequences. A long PCR using two genomic-specific primers flanking the insertion site will be performed to validate the length of the insertion. Since the cargo is inserted via HDR repair, it is anticipated that the cargo will be inserted in the designed direction as it is in the donor DNA, and the junctions will be free of additional mutations.

### Example 7. Use Cas12a-Trex2 Fusion to Achieve Efficient Targeted Chromosomal Translocation

This example is to demonstrate LbCas12a-Trex2 fusion, in conjunction with gRNAs in carefully designed orientations, may efficiently mediate the translocation of chromosomal arms between two non-homologous chromosomes. Soybean FAD2-1A gene on chromosome 10 and its paralog FAD2-1B gene on chromosome 20 is selected as the targets to demonstrate this design.

One gRNA will be designed to simultaneously target a sequence adjacent to a TTTV PAM and existing in both FAD2-1A and FAD2-1B. A second gRNA will be designed to simultaneously target another sequence adjacent to a TTTV PAM on the opposite strand and existing in both FAD2-1A and FAD2-1B. Co-expression of both gRNAs and LbCas12a-Trex2 fusion will generate gaps in both FAD2-1A and FAD2-1B, and the fusion molecules will remain bound to the genomic ends flanking the gaps. As illustrated in FIG. 4, at some frequency the dimerization of Trex2 will direct the exchange of the chromosomal arms between chromosomes 10 and 20. The chromosomal translocation will be identified by junction PCRs, each using one FAD2-1A-specific primer and one FAD2-1B-specific primer.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A fusion protein comprising a site-directed nuclease linked to a nonspecific end-processing enzyme.

2. The fusion protein of claim 1, wherein the site-directed nuclease comprises a CRISPR-associated nuclease.

3. The fusion protein of claim 2, wherein the CRISPR-associated nuclease is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof.

4. The fusion protein of claim 3, wherein the CRISPR-associated nuclease is a Cas9 enzyme.

5. The fusion protein of claim 3, wherein the CRISPR-associated nuclease is a Cas12a enzyme.

6. The fusion protein of claim 1, wherein the nonspecific end-processing enzyme is a nonspecific exonuclease.

7. The fusion protein of claim 6, wherein the nonspecific exonuclease is T5Exo, Trex2, E. coli exonuclease I, exonuclease III, exonuclease T, exonuclease IX, Exonuclease X, RecJ, Pol II, Pol III ε; WRN, MRE11, APE1, VDJP, RAD1, RAD9, p53, or Trex1.

8. The fusion protein of claim 1, wherein the nonspecific end-processing enzyme comprises an amino acid sequence having at least 90% identity to any one of SEQ ID NOs: 4, 5, 18, 19, 20, 22, or 58-74.

9. The fusion protein of claim 1, wherein the nonspecific end-processing enzyme is a monomer of a protein that dimerizes.

10. The fusion protein of claim 1, wherein the fusion protein comprises a linker located between the site-directed nuclease and the nonspecific end-processing enzyme.

11. The fusion protein of claim 10, wherein the linker comprises SEQ ID NO: 7.

12. The fusion protein of claim 1, wherein the fusion protein comprises a nuclear localization signal.

13. The fusion protein of claim 1, wherein the fusion protein comprises an amino acid sequence having at least 90% identity to any one of SEQ ID NOs: 50-57.

14. A recombinant nucleic acid encoding the fusion protein of claim 1.

15. A DNA construct comprising a promoter operably linked to the recombinant nucleic acid of claim 14.

16. The DNA construct of claim 15, wherein the promoter is at least one of an inducible promoter, a constitutive promoter, an egg cell-specific promoter, a pollen-specific promoter, or an apical meristem tissue-specific promoter.

17. The DNA construct of claim 15, wherein the promoter is a ubiquitin 4 promoter, an actin promoter, a tubulin promoter, a MADS box promoter, or a plant virus promoter.

18. A vector comprising the recombinant nucleic acid of claim 14.

19. A cell comprising the recombinant nucleic acid of claim 14.

20. The cell of claim 19, wherein the cell is a plant cell.

21. The cell of claim 20, wherein the plant cell is a maize plant cell, a soybean plant cell, a rice plant cell, a wheat plant cell, or a sunflower plant cell.

22. A method of editing a nucleic acid, the method comprising:
a. providing at least one fusion protein of claim 1;
b. providing the nucleic acid, wherein the nucleic acid comprises a first binding site, a second binding site, and a target region comprising a portion of the nucleic acid, wherein the first binding site is adjacent to a 5′ end of the target region and the second binding site is adjacent to the 3′ end of the target region; and
c. contacting the nucleic acid with the at least one fusion protein, wherein the at least one fusion protein specifically binds to the first binding site and the second binding site, thereby resulting in an edit to the target region of the nucleic acid.

23. The method of claim 22, wherein the site-directed nuclease of the at least one fusion protein comprises a CRISPR-associated nuclease and the method further comprises providing at least one first guide RNA and at least one second guide RNA, wherein the at least one first guide RNA comprises a nucleotide sequence having complementarity to the first binding site and the at least one second guide RNA comprises a nucleotide sequence having complementarity to the second binding site.

24. The method of claim 22, wherein the first binding site and the second binding site are on the same strand.

25. The method of claim 22, wherein the first binding site and the second binding site are on opposite strands.

26. The method of claim 22, wherein at least one of the first binding site or the second binding site are within the target region.

27. The method of claim 22, wherein both the first binding site and the second binding site are within the target region.

28. The method of claim 22, wherein neither the first binding site nor the second binding site are within the first target region.

29. The method of claim 22, the method further comprising providing a donor nucleic acid, wherein the donor nucleic acid comprises a third binding site, a fourth binding site, and a donor nucleotide region, wherein the third binding site is adjacent to a 5′ end of the donor nucleotide region and the fourth binding site is adjacent to the 3′ end of the donor nucleotide region and wherein the at least one fusion protein specifically binds to the third binding site and the fourth binding site.

30. The method of claim 29, wherein the site-directed nuclease of the at least one fusion protein comprises a CRISPR-associated nuclease and the method further comprises providing at least one third guide RNA and at least one fourth guide RNA, wherein the at least one third guide RNA comprises a nucleotide sequence having complementarity to the third binding site and the at least one fourth guide RNA comprises a nucleotide sequence having complementarity to the fourth binding site.

31. The method of claim 29, wherein the third binding site and the fourth binding site are on the same strand.

32. The method of claim 29, wherein the third binding site and the fourth binding site are on opposite strands.

33. The method of claim 29, wherein at least one of the third binding site or the fourth binding site are within the donor nucleotide region.

34. The method of claim 29, wherein both the third binding site and the fourth binding site are within the donor nucleotide region.

35. The method of claim 29, wherein neither the third binding site nor the fourth binding site are within the donor nucleotide region.

36. The method of claim 22, wherein the nucleic acid is a portion of a first chromosome.

37. The method of claim 29, wherein the donor nucleic acid is a portion of a donor template.

38. The method of claim 37, wherein the donor template is part of a plasmid or linear nucleic acid.

39. The method of claim 22, wherein the edit is an excision, an inversion, or a replacement of at least a portion of the target region.

40. The method of claim 36, wherein the donor nucleic acid is a portion of a second chromosome.

41. The method of claim 40, wherein the first chromosome and the second chromosome are homologous chromosomes or non-homologous chromosomes.

42. The method of claim 40, wherein the edit is a chromosomal rearrangement or a replacement of at least a portion of the target region.

43. The method of claim 42, wherein the chromosomal rearrangement is a reciprocal translocation or a non-reciprocal translocation.
